(19) United States
(12) Patent Application Publication
(10) Pub. No.: US 2019/0194148 A1
(43) Pub. Date: Jun. 27, 2019

(54) TETRAZOLE-CONTAINING 1,2-CYCLOPROPANE-CARBOXAMIDES

(71) Applicant: Bayer Pharma Aktiengesellschaft, Berlin (DE)

(72) Inventors: Stefan Bäurle, Berlin (DE); Jens Nagel, Daxweiler (DE); Andrea Rotgeri, Berlin (DE); Adam James Davenport, Abingdon (GB); Christopher Charles Stimson, Abingdon (GB)

(21) Appl. No.: 16/211,456

(22) Filed: Dec. 6, 2018

(30) Foreign Application Priority Data
Dec. 13, 2017 (EP) 17207116.9

(51) Int. Cl. C07D 257/04; C07D 403/12; C07D 401/10; C07D 409/10; C07D 401/14

(52) U.S. Cl. CPC C07D 257/04; C07D 403/12; C07D 401/14; C07D 409/10; C07D 401/10

(57) ABSTRACT

The present invention relates to N-(tetrazolylaryl) amide compounds of general formula (I) as described and defined herein, to pharmaceutical compositions and combinations comprising said compounds and to the use of said compounds for manufacturing a pharmaceutical composition for the treatment or prophylaxis of a disease, in particular of neurogenic disorder, as a sole agent or in combination with other active ingredients.

The present invention relates to tetrazole containing phenyl or pyridinyl compounds of general formula (I) as described and defined herein, to pharmacological compositions and combinations comprising said compounds and to the use of said compounds for manufacturing a pharmaceutical composition for the treatment or prophylaxis of a disease, in particular of Bradykinin B1 receptor associated disorders which are related to inflammation or at least partially driven by neurogenic events like diseases related to chronic pain or frequent pain conditions like but not restricted to osteoarthritis, rheumatoid arthritis, gout, inflammatory bowel disease, and endometriosis and diseases related to Bradykinin B1 receptor activation and/or up-regulation in affected tissue like but not restricted to asthma, fibrosis in various tissues or diabetes as a sole agent or in combination with other active ingredients.

### BACKGROUND OF THE INVENTION

The present invention relates to chemical compounds that antagonize the effects of human Bradykinin B1 receptor (Gene Name BDKRB1, Gene ID 623).

The Bradykinin B1 receptor is a membrane-bound G-protein coupled receptor, which is linked to a second messenger system that triggers increase of intracellular calcium concentrations. The main signalling pathway is linked to Gq protein and phospholipase C (Leeb-Lundberg, L. M. et al. (2005), Pharmacol Rev 57(1): 27-77). Activation of Bradykinin B1 receptor has been shown to be pro-algesic, pro-fibrotic, and proinflammatory while Bradykinin B1 receptor antagonists had clear anti-inflammatory and analgesic effects in various animal models (Gougat, J. B. et al. (2004), J Pharmacol Exp Ther 309(2): 661-669; Dias, J. P. et al. (2007), Br J Pharmacol 152(2): 280-287; Schuelert, N. et al. (2015), Eur J Pain 19(1): 132-142). As consequence of Bradykinin B1 receptor activity increased gene expression and protein levels of proinflammatory cytokines like e.g. Il-6 and Il-8 that attract and activate inflammatory leucocytes, increase of PGE2 (Prostaglandin 2) levels and therefore activation of the inflammation related prostaglandin pathway, phosphorylation and upregulation of TRPV1 (Transient Receptor Potential Vanilloid 1) receptors which are important mediators of pain transduction and induction of neurogenic inflammation (neuropeptide release in inflamed tissue) were observed (Phagoo, S. B. et al. (1999). Mol Pharmacol 56(2): 325-333; Westermann, D. et al. (2009), Diabetes 58(6): 1373-1381; Walsh, D. A. et al. (2006), Curr Drug Targets 7(8): 1031-1042; Farkas S. et al. (2011), Drugs of the Future 36(4): 301-319). Bradykinin B1 receptor agonists are endogenously produced by the activated kallikrein-kinin system. This system consists of circulating kininogens, the ubiquitous expressed proteolytic enzymes kallikreins which are activated by tissue damage, and kinins which are formed by activated kallikreins out of kininogens (Review Fincham, C. I. et al. (2009), Expert Opin Ther Pat 19(7): 919-941). These kinins (e.g. bradykinin, kalidin, des-Arg9-bradykinin, des-Arg10-kalidin) are proinflammatory peptides that mediate vascular and pain responses to tissue injury, with functions in cardiovascular homeostasis, contraction or relaxation of smooth muscle, inflammation and nociception. They exert most of their effects by interacting with two classes of G-protein-coupled receptors called Bradykinin receptor 1 and 2. The classification of the kinin receptors was originally achieved by means of pharmacological studies carried out at the end of the 1970s. During the 1990s, the existence of Bradykinin B1 receptor and B2 receptors was further confirmed through cloning and genetic deletion studies (Menke, J. G. et al. (1994), J Biol Chem 269(34): 21583-21586). The past 30 years of research on the kinin system has indicated that both Bradykinin B1 receptor and B2 receptor are involved in pain and inflammation (Leeb-Lundberg, L. M. et al. (2005), Pharmacol Rev 57(1): 27-77; Marceau, F. (2005), Trends Pharmacol Sci 26(3): 116-118; Marceau, F. (2004), Nat Rev Drug Discov 3(10): 845-852; Chen, J. J. et al. (2007), Expert Opin Ther Targets 11(1): 21-35).

It has been demonstrated that the B2 receptor is widely expressed in a constitutive manner throughout most mammalian tissues. In contrast, the Bradykinin B1 receptor is not constitutively expressed to a great extent under normal conditions, but is up-regulated under various inflammatory conditions such as asthma, arthritis and osteoarthritis, sepsis and type-1 diabetes, as well as by some neuropathological diseases such as epilepsy, stroke and multiple sclerosis. Bradykinin B1 receptor up-regulation can be induced for example by Il-1beta (Phagoo, S. B. et al. (1999), Mol Pharmacol 56(2): 325-333) and Bradykinin B2 receptor activation (NF-□B activation leading to IL-1□ expression in fibroblasts) (Leeb-Lundberg, L. M. et al. (2005), Pharmacol Rev 57(1): 27-77).

Once upregulated, the Bradykinin B1 receptor is expressed on neurons, macrophages, neutrophils, fibroblasts, smooth muscle cells and the vascular endothelium (Fincham, C. I. et al. (2009), Expert Opin Ther Pat 19(7): 919-941). Recent findings suggest that the Bradykinin B1 receptor expressed in the peripheral and in the central nervous system is involved in processing of inflammatory pain (Schuelert, N. et al. (2015). Eur J Pain 19(1): 132-142).

In contrast to Bradykinin B2 receptor and many other GPCRs (G protein-coupled receptors), the Bradykinin B1 receptor does not show agonist induced internalization or relevant desensitization (Prado, G. N. et al. (2002), J Cell Physiol 193(3): 275-286; Eisenbarth, H. et al. (2004), Pain 110(1-2): 197-204). Activation of Bradykinin B1 receptor triggers auto-induction of the receptor. This might lead to an augmentation of the inflammatory or pain-inducing processes.

Therefore, Bradykinin B1 receptor has been suggested to have a pivotal role including but not limited to several chronic diseases involving diabetes, fibrosis, inflammation, neuroinflammation, neurodegeneration, inflammatory pain, and neuropathic pain (Campos, M. M. et al. (2006), Trends Pharmacol Sci 27(12): 646-651; Wang, P. H. et al. (2009), Int Immunopharmacol 9(6): 653-657; Passos, G. F. et al. (2013), Am J Pathol 182(5): 1740-1749; Gobeil, F. et al. (2014), Peptides 52: 82-89; Huart, A. (2015), Front Pharmacol 6: 8). The contribution of Bradykinin B1 receptor activation in inflammation and pain processes is supported by the demonstration that Bradykinin B1 receptor knockout mice have a largely decreased response to nociceptive and proinflammatory stimuli (Ferreira, J. et al. (2001), Neuropharmacology 41(8):1006-1012; Ferreira, J. et al. (2005), J Neurosci 25(9): 2405-2412). The therapeutic impact of Bradykinin B1 receptor blockage for inflammation related diseases is supported further by the pharmacological properties of Bradykinin B1 receptor antagonists shown in many inflammatory and neuropathic pain models (Gougat, J. B. et al. (2004), J Pharmacol Exp Ther 309(2): 661-669; Fox, A. et al. (2005), Br J Pharmacol 144(7): 889-899).

The fact that Bradykinin B1 receptor expression is induced under disease conditions clearly raises the possibility that therapeutic use of Bradykinin B1 receptor antagonists should be devoid of undesired adverse effects. This property supports the suitability of Bradykinin B1 receptor antagonists for treatment of benign diseases like endometriosis due to the expected positive risk benefit ratio. The patient populations for nociceptive pain and neuropathic pain are large, and are driven by separate disease trends that necessitate pain relief. Chronic pain of moderate to severe intensity occurs in 19% of adult Europeans, seriously affecting the quality of their social and working lives (Breivik et al., Eur J Pain. 2006 May; 10(4):287-333.). Unfortunately, current treatments for pain are only partially effective, and many cause life-style altering, debilitating, and/or dangerous side effects. For example, non-steroidal anti-inflammatory drugs (NSAIDS) such as aspirin, ibuprofen, and indomethacin are moderately effective against inflammatory pain but they are also renally toxic, and high doses tend to cause gastrointestinal irritation, ulceration, bleeding, confusion and increased cardiovascular risk. Notably, Vioxx was withdrawn from the market in 2004 due to a risk of myocardial infarction and stroke. Patients treated with opioids frequently experience confusion and constipation, and long-term opioid use is associated with tolerance and addiction. Local anaesthetics such as lidocaine and mexiletine simultaneously inhibit pain and cause loss of normal sensation. In addition, when used systemically, local anaesthetics are associated with adverse cardiovascular effects. Thus, there is currently an unmet need in the treatment of chronic pain in general.

Especially in gynaecological therapy field, endometriosis is the disease associated with chronic pelvic pain severely affecting quality of life of the patients. Globally, approximately 11% of women aged 15-49 years are affected by endometriosis and additional 6% of women suffer from symptoms suggestive for endometriosis. Main symptoms of endometriosis are chronic or frequent pelvic pain, dyspareunia, dyschezia, dysuria and sub- or infertility. These symptoms severely impair quality of life of patients. Diagnosis of the disease involves a complete medical history, a physical examination and a laparoscopy. As an ultimate confirmation of endometriosis can only be made invasively and symptoms are often unspecific, the mean time from initial symptoms to diagnosis of endometriosis is about 7-10 years. Therefore, endometriosis is under-diagnosed and the number of affected women might be much higher than anticipated. Recently published EndoCost study demonstrated that cost of productivity loss of €6,298 per woman were double the healthcare cost of €3,113 per women, driven mainly by surgery and monitoring visit (Gao, X. et al. (2006), Fertil Steril 86(6): 1561-1572; Simoens S, et al. Hum Reprod (2012), 27(5):1292-9; De Graaff A, et al. (2013), Hum Reprod; 28(10): 2677-85).

Endometriosis is characterized by growth of endometrial tissue outside of the uterine cavity forming benign tumours (lesions) in the affected part of the body. Depending on lesion location and innervation severity of pain symptoms is observed. Up-regulation of various inflammation markers observed in the affected tissue and in the peritoneal tissue underline the inflammatory character of the disease (Stratton, P. et al. (2011), Hum Reprod Update 17(3): 327-346; Gao, X. et al. (2006), Fertil Steril 86(6): 1561-1572; Laux-Biehlmann et al. (2015), Trends Pharmacol Sci 36(5): 270-276).

The Bradykinin B1 receptor was identified in endometriosis lesion by immune-histological-chemical (IHC) staining (Yoshino et al. Journal of Reproductive Immunology 112 (2015) 121-140; www.proteinatlas.org) and analysis of m-RNA expression of Bradykinin B1 receptor in affected tissue shows a positive correlation to pain severity reported by endometriosis patients. The concept to treat endometriosis with Bradykinin B1 receptor antagonists is further supported by data describing a role of Bradykinin B1 receptors in affecting the outcome of an endometriosis mouse model (Jingwei, C. et al. (2015), J Tradit Chin Med 35(2): 184-191).

Suspected endometriosis is initially treated with non-steroidal anti-inflammatory drugs (NSAID) or combined oral contraceptives (COC) which are used off label. This procedure delays endometriosis diagnosis. Laparoscopy is the gold standard for endometriosis diagnosis which is performed when the initial treatment options fail. During laparoscopy, endometriotic lesions are ablated. However, this procedure is accompanied by a high recurrence rate. Approximately, 70% of treated patients have persistent symptoms that are not managed. Currently, there is no long-term medication available in COC/P (Combined Oral Contraceptives/Progestin) non-responder endometriosis patients in which COCs and progestins failed. Treatment with Gonadotropin Releasing Hormone (GnRH) agonists, which are used as second line therapy (without proof of being superior versus first line) are only approved for short-term treatment (6 months). After GnRH agonist application, systemic estradiol levels are suppressed up to 90% leading to chemical castration with menopausal side effects like bone mass loss and hot flushes. Therefore, new and long-term treatment options with reduced side-effects and high efficacy for endometriosis patients, in particular for patients with COC/P non-responder endometriosis, are urgently needed.

On this background the Bradykinin B1 receptor antagonists are of value for treatment of disorders which are related to inflammation or at least partially driven by neurogenic events like diseases related to chronic pain or frequent pain conditions like but not restricted to osteoarthritis (Kaufman, G. N. et al. (2011), Arthritis Res Ther 13(3): R76), rheumatoid arthritis (Cassim, B. et al. (2009), Rheumatology 48(5): 490-496), gout (Silva, C. R. et al. (2016), Ann Rheum Dis 75(1): 260-268), burn injuries and sunburn (Eisenbarth, H. et al. (2004), Pain 110(1-2): 197-204), inflammatory bowel disease, endometriosis (Yoshino et al. Journal of Reproductive Immunology 112 (2015) 121-140; Laux-Biehlmann et al. (2015), Trends Pharmacol Sci 36(5): 270-276; Jingwei, C. et al. (2015), J Tradit Chin Med 35(2): 184-191), pre-eclampsia (Moyes, A. J. et al. (2014), Hypertens Pregnancy 33(2): 177-190), diabetic neuropathy (Dias, J. P. et al. (2007), Br J Pharmacol 152(2): 280-287) including neuropathy related to diabetes type 1 and diabetes type 2, cardiac inflammation (Westermann, D. et al. (2009), Diabetes 58(6): 1373-1381), renal inflammation (Bascands, J. et al. (2009), Biochem Biophys Res Commun 386(2): 407-412), pancreatitis and diseases related to Bradykinin B1 receptor activation and/or up-regulation in affected tissue like but not restricted to asthma and cough (Bertram, C. M. et al. (2009), J Leukoc Biol 85(3): 544-552), atherosclerosis, diabetes (Dias, J. P. et al. (2012), J Cardiovasc Pharmacol 60(1): 61-69), adipositas including metabolic syndrome (Dias, J. P. et al. (2012), Diabetes Obes Metab 14(3): 244-253), diseases related to muscle atrophy including cachexia (Parreiras, E. S. L. T. et al. (2014), Clin Sci 127(3): 185-194) not limited to cancer cachexia, neuropathic pain (Luiz, A. P. et al. (2015), Neuroscience 300: 189-200), pruritus or itch (Hosogi, M. et al. (2006), Pain 126(1-3): 16-23), cancer (da Costa, P. L. et al. (2014), Cancer Lett 345(1): 27-38), neurodegenerative diseases such as amyotrophic lateral sclerosis (ALS) or Alzheimer's disease (Lacoste, et al. (2013) J Neuroinflammation 10: 57), fibrosis in cardiacs (Westermann, D. et al. (2009), Diabetes 58(6): 1373-1381), fibrosis in renal (Huart, A. et al. (2015), Front Pharmacol 6: 8) and fibrosis in lung tissues, overactive urinary bladder syndrome and cystitis (Forner, S. et al. (2012), Br J Pharmacol 167(8): 1737-1752 and Belichard, P. et al (1999), Br J Pharmacol 128(1):213-219), impaired or painful wound healing (Schremmer-Danninger, E. et al. (2004), Biol Chem 385(11): 1069-1076) and sepsis (Murugesan, P et al. (2016), J Infect Dis 213(4): 532-540).

Several Bradykinin B1 receptor antagonists are known from prior art (Expert Opinion on Therapeutic Patents (2012), 22:12, 1443-1452). Various approaches for finding new Bradykinin B1 receptor antagonists are described, in particular peptidic structures and small molecules. Especially, arylsulfonamides and so-called cyclopropyl-carboxamides as the two main types of small molecules were investigated during the last decade. WO2003/065789 (Merck) discloses bradykinin B1 receptor antagonists or inverse agonists of the following general formula

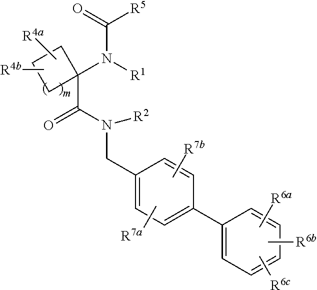

which are disclosed to be useful in the treatment or prevention of symptoms such as pain and inflammation associated with the bradykinin B1 pathway.

Merck was developing the bradykinin B1 receptor antagonist MK-0686 (structure shown below)

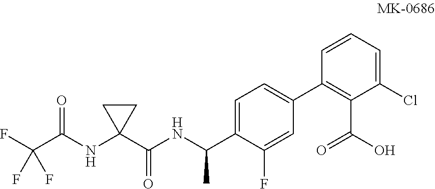 [SMILES: C[C@@H](NC(=O)C1(NC(=O)C(F)(F)F)CC1)c1ccc(-c2cccc(Cl)c2C(=O)O)cc1F]

for the potential treatment of pain and inflammation. Several phase II trials in subjects with osteoarthritis and with post-herpetic neuralgia were initiated. Merck accounted that the compound has a suboptimal pharmacokinetic profile due to metabolic liability.

Jerini AG, now Shire Group, investigated active Bradykinin B1 receptor antagonists, for example (see WO2009/036996)

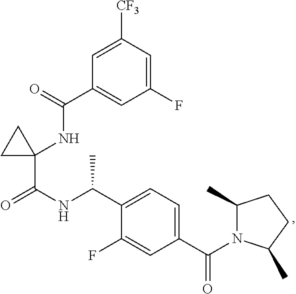 [SMILES: C[C@@H]1CC[C@H](C)N1C(=O)c1ccc([C@@H](C)NC(=O)C2(NC(=O)c3cc(F)cc(C(F)(F)F)c3)CC2)c(F)c1]

which was reported to have in addition to its activity and acceptable penetration profile reasonable aqueous solubility and pharmacokinetic profile in rat, whereas its human metabolic stability was still poor (Schaudt M, Locardi E, Zischinsky G, et al., Bioorg Med Chem Lett 2010;20:1225-8). Jerini exchanged the cyclopropyl-carboxamide moiety to a semicarbazide or to a five-membered diamino-heterocyclic ring or even to hydroxyureas without any explanation.

Starting with arylsulfonamide compounds as Bradykinin B1 receptor antagonists, Boehringer Ingelheim reported about several cyclopropyl-carboxamides out of their further development compounds like of the following structure

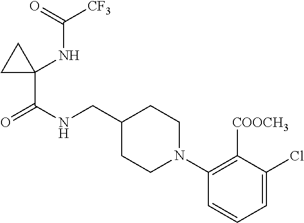 [SMILES: COC(=O)c1c(Cl)cccc1N1CCC(CNC(=O)C2(NC(=O)C(F)(F)F)CC2)CC1]

or related to that emerged with the highest binding affinity measured on human B1R-expressing CHO cells (Expert Opinion on Therapeutic Patents (2012), 22:12, 1443-1452).

In WO2012059776 Gedeon Richter reported about cyclopropyl-carboxamides of the following formula

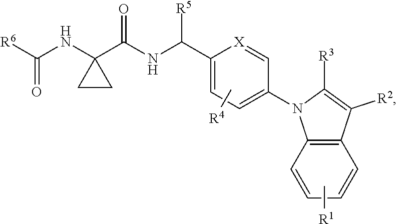

wherein R³ is selected from (1) —COOR; (2) —CN; (3) —CONR^{a}R^{b};

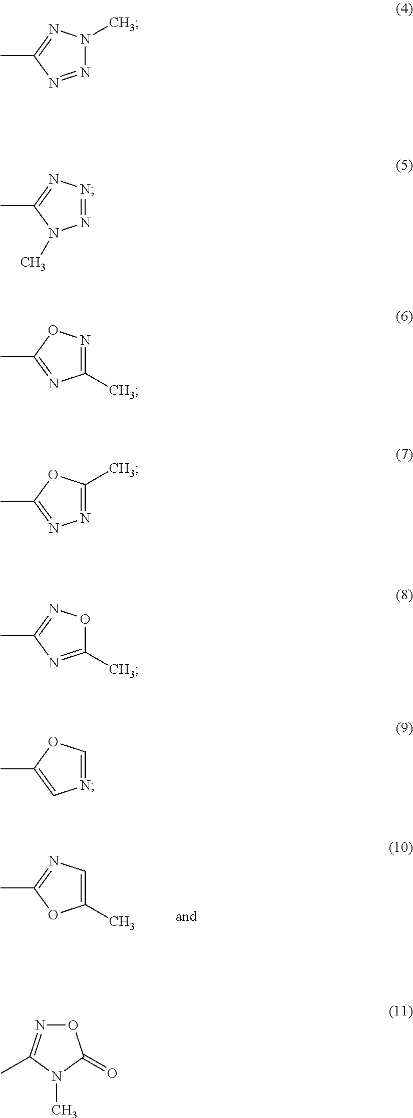

A majority of the compounds have a Kᵢ value below 20 nM on human recombinant Bradykinin receptors (expressed in CHO cells). Several indolyl compounds substituted with a tetrazol moiety are disclosed and represented by the following compound:

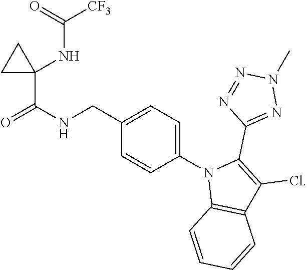 [SMILES: Cn1nnc(-c2c(Cl)c3ccccc3n2-c2ccc(CNC(=O)C3(NC(=O)C(F)(F)F)CC3)cc2)n1]

WO2005085227 (Smith Kline Beecham) discloses inhibitors of protein kinase B (PKB/Akt, PKB or Akt) of the formula

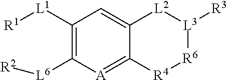

wherein
A is selected from: nitrogen, —C-halogen and —CH;
R¹ is selected from the group consisting of aryl, substituted aryl, cycloalkyl, substituted cycloalkyl, heterocycle and substituted heterocycle;
R² is selected from alkyl, substituted alkyl, cycloalkyl, substituted cycloalkyl, heterocycle, substituted heterocycle, and a cyclic or polycyclic aromatic ring,
L² is selected from the group consisting of a bond, —O—, heterocycle, —N(R⁵)—, —N(R⁵)C(O)—, —S—, —S(O)—, —S(O₂)—, and —C(O)N(R⁵)—; and
L¹ as well as L⁶ can be a bond, —O—, —N(R⁵)—, —S—, —S(O), —S(O₂)—, alkyl, and —N(R⁵)C(O)—. Neither L¹ nor L⁶ can be a heteroaryl or heterocyclic group. R⁴ is defined as hydrogen or halogen. The compounds are disclosed to be suitable for the treatment of cancer and arthritis. Tetrazole-substituted phenyl or pyridinyl compounds are not specifically disclosed.

In WO2012112567 (Georgetown University) small molecule inhibitors of ATP/GTP binding protein like 2 (AGBL2) of the formula

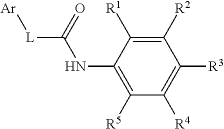

are disclosed wherein R² as well as R⁴ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, substituted or unsubstituted carbonyl, or substituted or unsubstituted carboxyl.
The compounds are disclosed to be used in methods for treating or preventing cancer and neurologic disorders. A tetrazole moiety as substituent at the benzene core structure is not specifically disclosed.

WO2009005638 (Merck) discloses a class of pyridinyl and pyrimidinyl derivatives of the formula

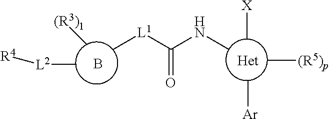

wherein the substituent Ar is aryl or heteroaryl, optionally substituted with halo, methyl, methoxy, halomethyl, amino, hydroxyl, C(O)OCH₃ or C(O)NHCH₃, X can be OH, SH or NH₂ and R⁵ is selected from H, OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(=O)O—, C₁-C₇ alkyl-C(=O)—, C₁-C₇ alkynyl, halo, hydroxyalkoxy, C₁-C₇ alkyl-NHSO₂—, C₁-C₇ alkyl-S O₂NH—, C₁-C₇ alkylsulfonyl, C₁-C₇ alkylamino or di(C₁-C₇)alkylamino. Neither X nor R⁵ can be a heteroaryl or heterocyclic group. Tetrazolyl is not specifically disclosed as substituent Ar. The compounds are disclosed to be used to treat cancer.

WO 2016168059 (DOW Agrosciences LLC) discloses compounds containing a 1,2-cyclopropyl of formula one

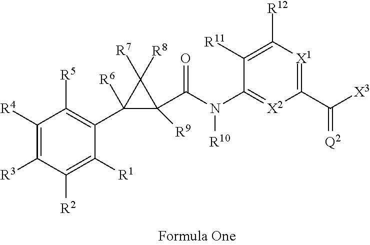

wherein Q² is S or O, and wherein X³ is selected from the group consisting of N(R¹⁵)(substituted or unsubstituted phenyl), N(R¹⁵) (substituted or unsubstituted heterocyclyl), and substituted or unsubstituted heterocyclyl. The compounds are disclosed as having pesticidal utility against pests in Phyla Arthropoda, Mollusca, and Nematoda. Furthermore processes to produce such compounds, intermediates used in such processes, pesticidal compositions containing the compounds, and processes of using such pesticidal compositions against such pests are also disclosed in WO 2016168059.

WO2012103583 (Bionomics) discloses 1,2-cyclopropyl-carboxamide compounds of formula (I)

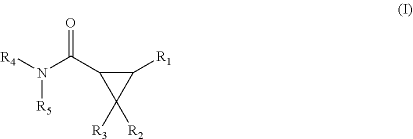

wherein R⁴ is selected from optionally substituted heteroaryl, optionally substituted heterocyclyl, or optionally substituted aryl, R⁵ is selected from hydrogen or optionally substituted alkyl, and R² is mandatory and cannot be hydrogen. Such compounds are disclosed to be useful in the positive modulation of the alpha 7 nicotinic acetylcholine receptor (α7nAChR). The disclosure of WO2012103583 also relates to the use of these compounds in the treatment or prevention of a broad range of diseases in which the positive modulation of α7nAChR is advantageous, including neurodegenerative and neuropsychiatric diseases and inflammatory diseases.

WO2007087066 (Vertex) discloses compounds and pharmaceutically acceptable compositions thereof, which are disclosed to be useful as modulators of ATP-Binding Cassette (“ABC”) transporters or fragments thereof, including Cystic Fibrosis Transmembrane Conductance Regulator (“CFTR”), having a benzamide core structure (I)

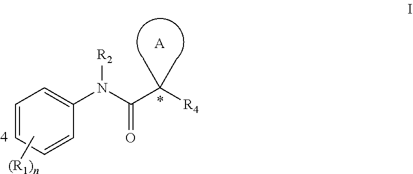

wherein ring A is an optionally substituted cycloaliphatic or an optionally substituted heterocycloaliphatic where the atoms of ring A adjacent to C* are carbon atoms. R4 is an optionally substituted aryl or an optionally substituted heteroaryl. R¹ is independently an optionally substituted C₁-C₆ aliphatic, an optionally substituted aryl, an optionally substituted heteroaryl, an optionally substituted C₃-C₁₀ membered cycloaliphatic or an optionally substituted 4 to 10 membered heterocycloaliphatic, carboxy, amido, amino, halo, or hydroxy, provided that at least one R¹ is an optionally substituted aryl or an optionally substituted heteroaryl and said R¹ is attached to the 3- or 4-position of the phenyl ring. Compounds in which the phenyl ring of the benzamide core structure is substituted with tetrazolyl are not disclosed.
So, the state of the art described above does not describe the specific compounds of general formula (I) of the present invention containing a tetrazol moiety as defined herein or an isomer, enantiomer, diastereomer, racemate, hydrate, solvate, or a salt thereof, or a mixture of same, as described and defined herein, and as hereinafter referred to as “compounds of the present invention”, or their pharmacological activity.

### SUMMARY OF THE INVENTION

The present invention covers N-(tetrazolylaryl) amides of general formula (I):

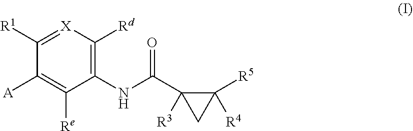

in which
A represents tetrazolyl which is attached to the rest of the molecule by the carbon atom;
R¹ represents
   phenyl,
   C₃-C₇-cycloalkyl,
   4- to 7-membered heterocycloalkyl containing 1 or 2 heteroatoms or heteroatom-containing groups selected from NH, —NR², N, O, S, SO and SO₂,
   5- to 7-membered lactam,
   5- or 6-membered heteroaryl, wherein said 5-membered heteroaryl contains 1, 2 or 3 heteroatoms selected from the group consisting of S, N, NH and O, and said 6-membered heteroaryl contains 1 or 2 N, or
   bicyclic 8- to 10-membered heteroaryl containing 1, 2 or 3 heteroatoms or heteratom-containing groups selected from the group consisting of N, NH, O, S, SO, and SO₂;
      wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, benzyl, NHR², N(R²)₂, NH(C₃-C₅-cycloalkyl), halogen, CN, NHSO₂R², SO₂R² or 4- to 7-membered heterocycloalkyl containing 1 or 2 heteroatoms or heteroatom-containing groups selected from NH, —NR², N, O, S, SO and SO₂, and
      wherein independently, if R¹ represents 4- to 7-membered heterocycloalkyl, 5- to 7-membered lactam, 5-membered heteroaryl or bicyclic 8- to 10-membered heteroaryl, each ring nitrogen atom, if present, of said R¹ is optionally substituted with a substituent R^{1b} wherein R^{1b} represents C₁-C₅-alkyl, C₃-C₅-cycloalkyl, —(C₁-C₃-alkyl)—(C₃-C₅-cycloalkyl), benzyl, SO₂R² or 4- to 7-membered heterocycloalkyl containing 1 or 2 heteroatoms or heteroatom-containing groups selected from NH, —NR², N, O, S, SO and SO₂, and
      if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)— (C₃-C₅-cycloalkyl), —OC₁—C₅-alkyl and —OC₃—C₅-cycloalkyl independently are optionally substituted with one or more substituents independently selected from the group consisting of OH, OR² and F, and
      if R^{1a} and/or R^{1b} represent 4- to 7-membered heterocycloalkyl, each carbon atom of said 4- to 7-membered heterocycloalkyl is independently optionally substituted with one or more substituents independently selected from the group consisting of OH, OR² and F;
R² represents C₁-C₅-alkyl optionally substituted with 1 to 5 fluorine atoms;
X represents CR^{c} or N;
R^{c} represents H, halogen, CN, OH, C₁-C₅-alkyl, or —OC₁—C₅-alkyl, wherein said C₁-C₅-alkyl and —OC₁—C₅-alkyl are optionally substituted with 1 to 5 fluorine atoms;
R^{d} represents H, halogen, CN, OH, C₁-C₅-alkyl, or —OC₁—C₅-alkyl wherein said C₁-C₅-alkyl and —OC₁—C₅-alkyl are optionally substituted with 1 to 5 fluorine atoms;
R^{e} represents H, halogen, or OH;
R³ and R⁴ independently represent H or C₁-C₃-alkyl, wherein said C₁-C₃-alkyl is optionally substituted with 1 to 3 fluorine atoms;
R⁵ represents
   C₁-C₅-alkyl,
   (CH₂)ₚ-phenyl,
   —(CH₂)ₚ-(C₅-C₇-cycloalkyl),
   5- or 6-membered heteroaryl, wherein said 6-membered heteroaryl contains 1 or 2 nitrogen atoms, and wherein said 5-membered heteroaryl contains 1, 2, or 3 heteroatoms or heteroatom-containing groups, the heteroatoms being independently selected from the group consisting of S, N, NH and O, or
   bicyclic 8- to 10-membered heteroaryl containing 1, 2 or 3 heteroatoms or heteroatom-containing groups selected from NH, N, O, S, SO, and SO₂,
   wherein said R⁵ is optionally substituted at one or more carbon atoms with 1 to 3 substituents which are the same or different and selected from the group consisting of C₁-C₅-alkyl, —OC₁—C₅-alkyl, halogen, OH and CN, wherein said C₁-C₅-alkyl and —OC₁-C₅-alkyl independently are optionally substituted with one or more substituents independently selected from the group consisting of OH, OR² and F, and
   wherein independently, if R⁵ represents 5-membered heteroaryl or bicyclic 8- to 10-membered heteroaryl, each ring nitrogen atom, if present, of said R⁵ independently is optionally substituted with C₁-C₅-alkyl, which is optionally substituted with OH, OR² or 1 to 5 fluorine atoms; and
p is 0 or 1;
or an isomer, enantiomer, diastereomer, racemate, hydrate, solvate, or a salt thereof, or a mixture of the same.

The present invention further relates to pharmaceutical compositions and combinations comprising said compounds, to use of said compounds for manufacturing a medicament for the treatment or prophylaxis of diseases or disorders and for the treatment of pains, which are associated with such diseases.

It has now been found, and this constitutes the basis of the present invention, that said compounds of the present invention have surprising and advantageous properties.

In particular, said compounds of the present invention have surprisingly been found to effectively inhibit Bradykinin B1 receptor and may therefore be used for the treatment or prophylaxis of following diseases and disorders:

Pain and inflammation, in particular any one of
visceral pain e.g. related to pancreatitis, interstitial cystitis, renal colic, or prostatitis, chronic pelvic pain, or pain related to infiltrating endometriosis;
neuropathic pain such as post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
central pain syndromes potentially caused by virtually any lesion at any level of the nervous system including but not limited to pain related to stroke, multiple sclerosis, and spinal cord injury; and
postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative and perioperative pain (including but not limited to general surgery, orthopaedic, and gynaecological surgery); and
acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis);
and diseases selected from or related to any one of:
gynaecological disorders and/or diseases, or effects and/or symptoms which negatively influence women health including endometriosis, uterine fibroids, pre-eclampsia, hormonal deficiency, spasms of the uterus, or heavy menstrual bleeding;
the respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
dermatology including pruritus, itch, inflammatory skin disorders including psoriasis, eczema, and atopic dermatitis;
affection of the joints or bones including rheumatoid arthritis, gout, osteoporosis, osteoarthritis, and ankylosing spondylitis;
affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
infection including HIV infection, and tuberculosis;
trauma associated with oedema including cerebral oedema, burns, sunburns, and sprains or fracture;
poisoning including aluminosis, anthracosis, asbestosis, chalicosis, ptilosis, siderosis, silicosis, tabacosis, and byssinosis uveitis,
diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, fat or muscle metabolism,
cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
cardio-vascular system including congestive heart failure, atherosclerosis, congestive heart failure, myocardial infarct, and heart fibrosis; and
other conditions including septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.
   Additionally, compounds of the present invention reduce the release of inflammation related cytokines like IL-6 and IL-8.

### DETAILED DESCRIPTION OF THE INVENTION

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or sulfur atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “one or a plurality up to the maximum possible amount”, e.g. if the term refers to the carbon atoms of a C₇-cycloalkyl, it relates to “1, 2, 3, 4, 5, 6 or 7”. In particular, “one or more” means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

The term “comprising” when used in the specification includes but is not restricted to “consisting of”.

The terms as mentioned in the present text have preferably the following meanings:

The term “halogen atom”, “halogen”, “halo-” or “Hal-” is to be understood as meaning a fluorine, chlorine, bromine or iodine atom, preferably a fluorine or a chlorine atom.

The term “C₁-C₅-alkyl” means a linear or branched, saturated, monovalent hydrocarbon group having 1, 2, 3, 4 or 5 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl or 1,1-dimethylpropyl group, or an isomer thereof “C₁-C₅-alkyl, optionally substituted with 1 to 5 fluorine atoms” is, for example, —CF₃, —CF₂H, —CH₂CF₃, —CH(CH₃)CF₃, —CH₂CHF₂, —CH₂CH₂CF₃, —CH₂CF₂CF₃, —CF₂CH₂CH₃, —CH₂CH₂CH₂CF₃, —CH₂CH₂CF₂CF₃, —CH(CF₃)CH₂CH₃, —CF₂CH₂CH₂CH₃, or —CH₂C(CF₃)(CH₃)₂.

The term “C₁-C₃-alkyl” means a linear or branched, saturated, monovalent hydrocarbon group having 1, 2 or 3 carbon atoms (“C₁-C₃-alkyl”), e.g. a methyl, ethyl, n-propyl or isopropyl group.

The term “—OC₁—C₅-alkyl” means a linear or branched, saturated, monovalent group which is attached through an oxygen atom, and in which the term “C₁-C₅-alkyl” is as defined supra, e.g. a methoxy, ethoxy, n-propoxy, isopropoxy, n-butoxy, sec-butoxy, isobutoxy, tert-butoxy, pentyloxy or isopentyloxy, or an isomer thereof. The hyphen at the beginning of the group indicates the point of attachment of said OC₁-C₅-alkyl group to the rest of the molecule.

“C₃-C₇-cycloalkyl” is to be understood as meaning a saturated, monovalent, monocyclic or bicyclic hydrocarbon ring, which contains 3, 4, 5, 6 or 7 carbon atoms. Said C₃-C₇-cycloalkyl group is for example a monocyclic hydrocarbon ring, e.g. a cyclopropyl, cyclobutyl, cyclopentyl, cyclohexyl or cycloheptyl group, or a bicyclic hydrocarbon ring, e.g. a bicyclo[2.2.1]heptanyl or bicyclo[3.2.0]heptanyl group. Particularly, said ring contains 3, 4 or 5 carbon atoms (“C₃-C₅-cycloalkyl”) or 5, 6 or 7 carbon atoms (“C₅-C₇-cycloalkyl”).

The term “bicyclic cycloalkyl” includes by definition spirocycloalkyl, bridged and fused bicycloalkyl groups.

The term “spirocycloalkyl” means a saturated, monovalent bicyclic hydrocarbon group in which the two rings share one common ring carbon atom, and wherein said bicyclic hydrocarbon group contains 5, 6, or 7 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl or spiro[2.4]heptyl.

The term “fused bicycloalkyl” means a bicyclic, saturated hydrocarbon ring with 6 or 7 ring atoms in total, in which the two rings share two adjacent ring atoms.

Said fused cycloalkyl group is, for example, a bicyclo[3.1.0]hexanyl or bicyclo[3.2.0]heptanyl group.

The term “bridged bicycloalkyl” means a bicyclic, saturated hydrocarbon ring with 6 or 7 ring atoms in total, in which the two rings share two common ring atoms which are not adjacent. Said bridged cycloalkyl group is, for example, bicyclo[2.1.1]hexanyl or bicyclo[2.2.1]heptanyl group.

The term “—(C₁-C₃-alkyl)—(C₃-C₅-cycloalkyl)” is to be understood as a C₃-C₅-cycloalkyl group as defined above which is attached through any carbon atom of said C₃-C₅-cycloalkyl group to any atom of the C₁-C₃-alkyl group as defined above. The hyphen at the beginning of the group indicates the point of attachment of said (C₁-C₃-alkyl)—(C₃-C₅-cycloalkyl) group to the rest of the molecule. Said (C₁-C₃-alkyl)—(C₃-C₅-cycloalkyl) groups are, for example, cyclopropylmethyl, cyclobutylmethyl, cyclopentylmethyl, 2-cyclopropylethyl, 1-cyclopropylethyl, 2-cyclobutylethyl or 1-cyclobutylethyl.

The term “—OC₃—C₅-cycloalkyl” means a saturated, monovalent, monocyclic group, which contains 3, 4 or 5 carbon atoms, in which the term “C₃-C₅-cycloalkyl” is defined supra, e.g. a cyclopropyloxy, cyclobutyloxy or cyclopentyloxy group.

The term “heterocycloalkyl” is to be understood as meaning a saturated, monovalent, monocyclic or bicyclic hydrocarbon ring with the number of ring atoms as specified in which one or two ring atoms of the hydrocarbon ring is/are replaced by one or two heteroatoms or heteroatom-containing groups independently selected from NH, —NR², N, O, S, SO and SO₂, wherein R² represents C₁-C₅-alkyl optionally substituted with 1 to 5 fluorine atoms. Said heterocycloalkyl can be connected through a carbon or a nitrogen atom, if said nitrogen atom is present.

“4- to 7-membered heterocycloalkyl” in the context of the invention means a monocyclic or bicyclic, saturated heterocycle with 4, 5, 6 or 7 ring atoms in total, which contains one or two identical or different ring heteroatoms or heteroatom-containing groups from the series NH, —NR², N, O, S, SO and SO₂, wherein R² represents C₁-C₅-alkyl optionally substituted with 1 to 5 fluorine atoms. Said 4- to 7-membered heterocycloalkyl can be bound via a ring carbon or nitrogen atom to the rest of the molecule.

Examples for monocyclic heterocycloalkyl groups are azetidinyl, oxetanyl, thietanyl, pyrrolidinyl, pyrazolidinyl, imidazolidinyl, tetrahydrofuranyl, thiolanyl, 1,1-dioxidothiolanyl, 1,2-oxazolidinyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, 1,2-oxazinanyl, morpholinyl, thiomorpholinyl, 1,1-dioxidothiomorpholinyl, azepanyl, 1,4-diazepanyl, and 1,4-oxazepanyl.

Particularly, without being limited thereto, said heterocycloalkyl can be a 4-membered ring, such as an azetidinyl, oxetanyl or thietanyl, or a 5-membered ring, such as tetrahydrofuranyl, dioxolinyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, or a 7-membered ring, such as a azepanyl, 1,4-diazepanyl or 1,4-oxazepanyl, for example.

The term “bicyclic heterocycloalkyl” includes by definition heterospirocycloalkyl, fused and bridged heterobicycloalkyl groups.

The term “heterospirocycloalkyl” means a bicyclic, saturated heterocycle with 6 or 7 ring atoms in total, in which the two rings share one common ring carbon atom, which “heterospirocycloalkyl” contains one or two identical or different ring heteroatoms or heteroatom-containing groups from the series: NH, —NR², N, O, S, SO and SO₂, wherein R² represents C₁-C₅-alkyl optionally substituted with 1 to 5 fluorine atoms; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[2.4]-heptanyl, azaspiro[3.3]heptyl, oxazaspiro[3.3]heptyl, thiazaspiro[3.3]heptyl, oxaspiro[3.3]heptyl, diazaspiro[3.3]heptyl or thiazaspiro[3.3]heptyl, or one of the further homologous scaffolds such as spiro[2.3]-, spiro[2.4]-, spiro[3.3]-.

The term “fused heterocycloalkyl” means a bicyclic, saturated heterocycle with 6 or 7 ring atoms in total, in which the two rings share two adjacent ring atoms, which “fused heterocycloalkyl” contains one or two identical or different ring heteroatoms or heteroatom-containing groups from the series: NH, —NR², N, O, S, SO and SO₂, wherein R² represents C₁-C₅-alkyl optionally substituted with 1 to 5 fluorine atoms; it being possible for said fused heterocycloalkyl group to be attached to the rest of the molecule via any one of the carbon atoms or, if present, a nitrogen atom.

Said fused heterocycloalkyl group is, for example, 3-azabicyclo[3.1.0]hexanyl or 3-azabicyclo[3.2.0]heptanyl.

The term “bridged heterocycloalkyl” means a bicyclic, saturated heterocycle with 6 or 7 ring atoms in total, in which the two rings share two common ring atoms which are not adjacent, which “bridged heterocycloalkyl” contains one or two identical or different ring heteroatoms or heteroatom-containing groups from the series: NH, —NR², N, O, S, SO and SO₂, wherein R² represents C₁-C₅-alkyl optionally substituted with 1 to 5 fluorine atoms; it being possible for said bridged heterocycloalkyl group to be attached to the rest of the molecule via any one of the carbon atoms, except the bridgehead carbon atoms, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl or diazabicyclo[2.2.1]heptyl.

The term “5- to 7-membered lactam” means cyclic amides of amino carboxylic acids, having a 1-azacycloalkan-2-one structure, or analogues having unsaturation or heteroatoms replacing one or more carbon atoms of the ring having a ring size of 5, 6 or 7 ring system atoms. In particular said “5- to 7-membered lactam” means a γ-lactam (pyrrolidinyl), a δ-lactam (delta-lactam), and an ε-lactam (epsilon-lactam).

The term “heteroaryl” is understood as meaning a monovalent, monocyclic or bicyclic hydrocarbon ring system with at least one aromatic ring and wherein one, two or three ring atoms of the monovalent, monocyclic or bicyclic hydrocarbon ring system is/are replaced by one, two or three heteroatoms or heteroatom-containing groups independently selected from NH, N, O, S, SO and SO₂. The number of ring system atoms is as specified.

“5- or 6-membered heteroaryl” is understood as meaning a heteroaryl having 5 or 6 ring atoms and wherein one, two or three ring atoms of a monovalent 5-membered hydrocarbon ring system is/are replaced by one, two or three heteroatoms or heteroatom-containing groups independently selected from S, N, NH and O; and wherein one or two ring atoms of a monovalent 6-membered hydrocarbon ring system is/are replaced by one or two nitrogens.

The said 5-membered heteroaryl can be connected through a carbon or a nitrogen atom, if said nitrogen atom is present.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl or thiadiazolyl; or a 6-membered heteroaryl group, such as, for example, pyridinyl, pyridazinyl, pyrimidinyl or pyrazinyl.

In general, and unless otherwise mentioned, the term “heteroaryl” includes all possible isomeric forms thereof, e.g. tautomers and positional isomers with respect to the point of linkage to the rest of the molecule. Thus, to give some illustrative non-restricting examples, the term pyridinyl includes pyridin-2-yl, pyridin-3-yl, and pyridin-4-yl; or the term pyrimidinyl includes pyrimidin-2-yl, pyrimidin-4-yl and pyrimidin-5-yl; or the term pyrazolyl includes 1H-pyrazolyl; or the term imidazolyl includes 1H-imidazolyl and 4H-imidazolyl; the term thiophenyl includes 2-thiophenyl and 3-thiophenyl; or the term thiazolyl includes 1,3-thiazol-5-yl, 1,3-thiazol-4-yl and 1,3-thiazol-2-yl.

“Bicyclic 8- to 10-membered heteroaryl” is understood as meaning a bicyclic heteroaryl having 8, 9 or 10 ring atoms with at least one aromatic ring and wherein one, two or three ring atoms of a monovalent, 8- to 10-membered bicyclic hydrocarbon ring system is/are replaced by one, two or three heteroatoms or heteroatom-containing groups independently selected from NH, N, O, S, SO and SO₂.

The said bicyclic 8- to 10-membered heteroaryl can be connected through a carbon or a nitrogen atom, if said nitrogen atom is present.

Particularly, bicyclic heteroaryl is selected from for example, benzofuranyl, benzothienyl, benzothiazolyl, thienopyridinyl, thienopyrimidinyl, benzoxazolyl, benzisoxazolyl, benzimidazolyl, benzotriazolyl, benzothiadiazolyl, indazolyl, indolyl, isoindolyl, etc. or for example, quinolinyl, quinazolinyl, isoquinolinyl, etc.; indolizinyl, or cinnolinyl, phthalazinyl, quinazolinyl, and quinoxalinyl, etc.

The term “C₁-C₃” as used throughout this text is to be understood as meaning a group having a finite number of carbon atoms of 1 to 3, i.e. 1, 2, or 3 carbon atoms, e.g. in the context of the definition of “C₁-C₃-alkyl”, it is to be understood as meaning an alkyl group having a finite number of carbon atoms of 1 to 3, i.e. 1, 2, or 3 carbon atoms. It is to be understood further that said term “C₁-C₃” is to be interpreted as any sub-range comprised therein, e.g. C₁-C₂, or C₂-C₃.

The term “C₁-C₅” as used throughout this text is to be understood as meaning a group having a finite number of carbon atoms of 1 to 5, i.e. 1, 2, 3, 4, or 5 carbon atoms, e.g. in the context of the definition of “C₁-C₅-alkyl”, it is to be understood as meaning an alkyl group having a finite number of carbon atoms of 1 to 5, i.e. 1, 2, 3, 4, or 5 carbon atoms. It is to be understood further that said term “C₁-C₅” is to be interpreted as any sub-range comprised therein, e.g. C₁-C₅, C₂-C₅, C₃-C₄, C₂-C₃, C₂-C₄, or C₁-C₄.

The term “C₁-C₃” as used in the context of the definition “—OC₁-C₃-alkyl” is to be understood as meaning an alkyl group, having a finite number of carbon atoms of 1 to 3, i.e. 1, 2 or 3 carbon atoms.

Similarly, the mentioned above applies to “C₁-C₄-alkyl”, “C₁-C₃-alkyl”, “C₁-C₃-alkoxy”, “C₁-C₂-alkyl” or “C₁-C₂-alkoxy”.

Further, as used herein, the term “C₃-C₇”, as used throughout this text, is to be understood as meaning a group having a finite number of carbon atoms of 3 to 7, i.e. 3, 4, 5, 6 or 7 carbon atoms, e.g. in the context of the definition of “C₃-C₇-cycloalkyl”, it is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, or C₅-C₇; particularly C₃-C₆.

Furthermore, as used herein, the term “C₃-C₅”, as used in the present text, e.g. in the context of the definition of “C₃-C₅-cycloalkyl”, means a cycloalkyl group having a finite number of carbon atoms of 3 to 5, i.e. 3, 4 or 5 carbon atoms.

When a range of values is given, said range encompasses each value and sub-range within said range.
For example:
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C8-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [trifluoromethyl)sulfonyl]oxy, [(nonafluorobutyl)-sulfonyl]oxy, (phenylsulfonyl)oxy, [(4-methylphenyl)sulfonyl]oxy, [(4-bromo-phenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethyl-phenyl)sulfonyl]oxy, [(4-tert-butylphenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998, which is incorporated herein by reference.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271] and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases, deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102; both incorporated herein by reference). In other cases, the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208; incorporated herein by reference) and Odanacatib (K. Kassahun et al., WO2012/112363; incorporated herein by reference) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993; incorporated herein by reference). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel O D and Chiracel O J among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976), thereby incorporated herein.

Further, the compounds of the present invention may exist as tautomers.

The present invention includes all possible tautomers of the compounds of the present invention as single tautomers, or as any mixture of said tautomers, in any ratio.

The term “tetrazolyl” as used in the context of the definition A in the general formula (I) is to be understood as both 1H- and 2H-tautomers.

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example. The 1,2-cyclopropylamides of the invention have to be understood, unless stated otherwise, as relating to both R³ and R⁴ cis and trans isomers, as either single entantiomers or a mixture of enantiomers. Preferred are mixtures of trans enantiomers, if not stated otherwise.

The present invention also relates to useful forms of the compounds as disclosed herein, such as hydrates, solvates, and salts, in particular pharmaceutically acceptable salts.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19, incorporated herein by reference. A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Unless otherwise indicated, the compounds of the present invention are also referred to isomers, enantiomers, diastereomers, racemates, hydrates, solvates, a salt thereof, or a mixture of same.

As used herein, the term “in vivo hydrolysable ester” is understood as meaning an in vivo hydrolysable ester of a compound of the present invention containing a carboxy or hydroxy group, for example, a pharmaceutically acceptable ester that is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention. An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorph, in any ratio.

The present invention also relates to useful forms of the compounds as disclosed herein, such as hydrates, solvates, and salts, in particular pharmaceutically acceptable salts.

Where the plural form of the word compounds, salts, polymorphs, hydrates, solvates and the like, is used herein, this is taken to mean also a single compound, salt, polymorph, isomer, hydrate, solvate or the like.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19, incorporated herein by reference. A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

In accordance with an aspect, the present invention covers compounds of general formula (I), wherein
A represents tetrazolyl which is attached to the rest of the molecule by the carbon atom.

In accordance with an aspect, the present invention covers compounds of general formula (I) wherein
R¹ represents
   phenyl,
   5- to 7-membered lactam,
   5- or 6-membered heteroaryl, wherein said 5-membered heteroaryl contains 1, 2 or 3 heteroatoms or heteroatom-containing groups selected from the group consisting of S, N, NH, and O, and wherein said 6-membered heteroaryl contains 1 or 2 nitrogen atoms, or
   bicyclic 9- or 10-membered heteroaryl containing 1, 2 or 3 heteroatoms or heteratom-containing groups selected from NH, N, O, S, SO and SO₂;
wherein R¹ is optionally substituted as defined in formula (I).

Also preferred are compounds of general formula (I), wherein
R¹ represents
   phenyl,
   5- to 7-membered lactam,
   5- to 6-membered heteroaryl, wherein said 5-membered heteroaryl contains 1, 2 or 3 heteroatoms or heteroatom-containing groups selected from the group consisting of S, N, NH and O, and wherein said 6-membered heteroaryl contains 1 or 2 nitrogen atoms, or
   bicyclic 9- to 10-membered heteroaryl containing 1, 2 or 3 heteroatoms or heteratom containing groups selected from the group consisting of NH, N, O, S, SO and SO₂;
   wherein said R¹ is optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently, if R¹ represents 5- to 7-membered lactam, 5-membered heteroaryl or bicyclic 9- or 10-membered heteroaryl, each ring nitrogen atom, if present, of said R¹ is optionally substituted with 1 substituent R^{1b}, wherein R^{1b} represents C₁-C₅-alkyl, C₃-C₅-cycloalkyl, or —(C₁-C₃-alkyl)—(C₃-C₅-cycloalkyl), and
   if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents
   phenyl,
      wherein said R¹ is optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁-C₅-alkyl, —OC₃—C₅-cycloalkyl, halogen or CN, and
      if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents
   phenyl,
   wherein said R¹ is optionally substituted at one or more carbon atoms with 1 or 2 substituents selected from the group consisting of methyl, methoxy, ethoxy, methoxymethyl, trifluoromethoxy, and a halogen, particular Cl.
   Also preferred are compounds of general formula (I), wherein
R¹ represents
   phenyl,
   wherein said R¹ is optionally substituted at two carbon atoms with 1 substituent at each substituted carbon atom, wherein the two substituents are independently selected from the group of combinations consisting of dimethoxy (methoxy and methoxy), ethoxy and methoxy, methoxymethyl and methyl, methyl and methoxy, and methoxy and halogen, in particular Cl.
   Also preferred are compounds of general formula (I), wherein
R¹ represents
   5- to 7-membered lactam,
      wherein said R¹ is optionally substituted at one or more carbon atoms with 1 or 2 substituents
      R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
      wherein the ring nitrogen atom of said 5- to 7-membered lactam is optionally substituted with 1 substituent R^{1b}, wherein R^{1b} represents C₁-C₅-alkyl, C₃-C₅-cycloalkyl, or —(C₁-C₃-alkyl)—(C₃-C₅-cycloalkyl), and
      if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents
   5-membered lactam,
      wherein said R¹ is optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
      wherein the ring nitrogen atom of said 5-membered lactam is optionally substituted with 1 substituent R^{1b}, wherein R^{1b} represents C₁-C₅-alkyl, C₃-C₅-cycloalkyl, or —(C₁-C₃-alkyl)—(C₃—C₅-cycloalkyl), and
      if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents
   5-membered lactam,
      wherein said R¹ is optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
      wherein the ring nitrogen atom of said 5-membered lactam links the lactam to the rest of the molecule, and
      wherein said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents oxopyrrolidinyl, in particular 2-oxopyrrolidin-1-yl,
   wherein said R¹ is optionally substituted at one carbon atom with a C₁-C₅-alkyl, in particular ethyl,
   wherein the ring nitrogen atom of said 5-membered lactam links the lactam to the rest of the molecule.
Also preferred are compounds of general formula (I), wherein
R¹ represents 3-ethyl-2-oxopyrrolidin-1-yl.
   Also preferred are compounds of general formula (I), wherein
R¹ represents
   6-membered heteroaryl containing 1 or 2 nitrogen atoms, in particular pyridinyl, or pyrimidinyl,
   wherein said R¹ is optionally substituted at one or more carbon atoms with 1 or 2 substituents R^{1a} which are the same or different,
   wherein R^{1a} represents C₁-C₅-alkyl, C₃-C₅-cycloalkyl, —(C₁-C₃-alkyl)—(C₃-C₅-cycloalkyl), —OC₁-C₅-alkyl, —OC₃—C₅-cycloalkyl, halogen or CN, and
   wherein said C₁-C₅-alkyl, C₃-C₅-cycloalkyl, —(C₁-C₃-alkyl)—(C₃-C₅-cycloalkyl), —OC₁-C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents pyridinyl, in particular pyridin-3-yl, optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN,
   wherein the substituent or at least one of said substituents R^{1a} is preferably positioned para to the carbon atom which links the pyridinyl, in particular pyridin-3-yl, to the rest of the molecule, and
   wherein said C₁-C₅-alkyl, C₃-C₅-cycloalkyl, —(C₁-C₃-alkyl)—(C₃-C₅-cycloalkyl), —OC₁-C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents pyridinyl, in particular pyridin-3-yl, substituted at one or more carbon atoms with 1 or 2 substituents which are the same or different selected from the group consisting of methoxy, ethoxy, isopropoxy, and trifluoromethyl, and
   wherein the substituent or at least one of said substituents is preferably positioned para to the carbon atom which links the pyridinyl, in particular pyridin-3-yl, to the rest of the molecule.

Also preferred are compounds of general formula (I), wherein
R¹ represents pyrimidinyl, in particular pyrimidin-5-yl, optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁-C₅-alkyl, —OC₃—C₅-cycloalkyl, halogen or CN,
   wherein the substituent or at least one of said substituents R^{1a} is preferably positioned para to the carbon atom which links the pyrimidinyl, in particular pyrimidin-5-yl, to the rest of the molecule,
   wherein if R^{1a} represents C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl, said C₁-C₅-alkyl, C₃-C₅-cycloalkyl, —(C₁-C₃-alkyl)—(C₃-C₅-cycloalkyl), —OC₁C₅-alkyl or —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents pyrimidinyl, in particular pyrimidin-5-yl, optionally substituted at one or more carbon atoms with 1 or 2 substituents R^{1a} being OC₁-C₅-alkyl,
   wherein the OC₁-C₅-alkyl is preferably positioned parato the carbon atom which links the pyrimidinyl, in particular pyrimidin-5-yl, to the rest of the molecule,
   wherein said —OC₁—C₅-alkyl is optionally substituted with one or more substituents independently selected from the group consisting of OH, OR² and F, and
R² has the same meaning as defined herein for general formula (I).
   Also preferred are compounds of general formula (I), wherein
R¹ represents pyrimidinyl, in particular pyrimidin-5-yl, substituted with ethoxy in para position to the carbon atom which links the pyrimidinyl, in particular pyrimidin-5-yl, to the rest of the molecule.
   Also preferred are compounds of general formula (I), wherein
R¹ represents a 5-membered heteroaryl containing 1, 2 or 3 heteroatoms or heteroatom-containing groups independently selected from the group consisting of S, N, NH, and O, in particular pyrazolyl, imidazolyl, thiophenyl, thiazolyl, oxadiazolyl, thiadiazolyl, or triazolyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents pyrazolyl, in particular pyrazol-1-yl or pyrazol-4-yl, optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents pyrazolyl, in particular pyrazol-1-yl or pyrazol-4-yl, optionally substituted at one or more carbon atoms with 1 or 2 substituents R^{1a} which are the same or different wherein R^{1a} represents C₁-C₅-alkyl or C₃-C₅-cycloalkyl,
   wherein R^{1a} is optionally substituted with one or more substituents independently selected from the group consisting of OH, OR² and F, and
R² has the same meaning as defined herein for general formula (I).
   Also preferred are compounds of general formula (I), wherein
R¹ represents pyrazol-1-yl optionally substituted at any carbon atom with a substituent selected from the group consisting of methyl, butyl, in particular tert-butyl, and trifluoromethyl.
   Also preferred are compounds of general formula (I), wherein
R¹ represents pyrazol-1-yl optionally substituted at the carbon atom at position 3 or 4 with a substituent selected from the group consisting of tert-butyl, and trifluoromethyl.
   Also preferred are compounds of general formula (I), wherein
R¹ represents pyrazol-4-yl optionally substituted at a nitrogen atom with 1 substituent R^{1b}, wherein R^{1b} represents C₁-C₅-alkyl or C₃-C₅-cycloalkyl, in particular methyl or cyclobutyl. Preferably, said R^{1b} is at the ring nitrogen at position 1 of the pryazol-4-yl.
   Also preferred are compounds of general formula (I), wherein
R¹ represents imidazolyl, in particular imidazol-1-yl, optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents imidazolyl, in particular imidazol-1-yl, optionally substituted at one or more carbon atoms with 1 or 2 substituents R^{1a} which are the same or different wherein R^{1a} represents C₁-C₅-alkyl, and
   said C₁-C₅-alkyl being optionally substituted with one or more substituents independently selected from the group consisting of OH, OR² and F, in particular R^{1a} being unsubstituted tert-butyl, and
R² has the same meaning as defined herein for general formula (I).
   Also preferred are compounds of general formula (I), wherein
R¹ represents imidazolyl, in particular imidazol-1-yl, optionally substituted at one or more carbon atoms with 1 or 2 substituents R^{1a} which are the same or different wherein R^{1a} represents C₁-C₅-alkyl, and
   said C₁-C₅-alkyl being optionally substituted with one or more substituents independently selected from the group consisting of OH, OR² and F, in particular R¹ being unsubstituted tert-butyl,
   wherein said C₁-C₅-alkyl, in particular tert-butyl, is in particular at the carbon atom at position 4 of said imidazol-1-yl, and
R² has the same meaning as defined herein for general formula (I).
   Also preferred are compounds of general formula (I), wherein
R¹ represents a 9-membered bicyclic heteroaryl containing 1, 2 or 3 heteroatoms or heteroatom-containing groups selected from NH, N, O, S, SO and SO₂, in particular benzopyrazolyl, benzimidazolyl, benzothiophenyl, benzothiazolyl, benzoxadiazolyl, benzothiadiazolyl, thiazolopyridinyl, imidazopyridinyl, oxazolopyridinyl, furopyridinyl, benzofuranyl, or thienopyridynyl, more particular benzothiophenyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁-C₅-alkyl, —OC₃—C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R¹ represents benzothiophenyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁-C₅-alkyl and —OC₃—C₅-cycloalkyl independently are optionally substituted with one or more substituents independently selected from the group consisting of OH, OR² and F, and
R² has the same meaning as defined herein for general formula (I).
   In accordance with an aspect, the present invention covers compounds of general formula (I), wherein
R¹ represents phenyl, pyridinyl, pyrimidinyl, pyrazolyl, imidazolyl, benzothiophenyl, or pyrrolidinyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently, if R¹ represents pyrazolyl, imidazolyl, or pyrrolidinyl, any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   In another preferred embodiment, the invention relates to compounds of general formula (I), wherein
R¹ represents phenyl, pyridinyl, pyrazolyl, imidazolyl, benzothiophenyl, or pyrrolidinyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently, if R¹ represents pyrazolyl, imidazolyl, or pyrrolidinyl any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I).
   Also preferred are compounds of general formula (I), wherein
R² represents C₁-C₅-alkyl optionally substituted with 1 to 3 fluorine atoms, in particular methyl, ethyl, propyl, or butyl, each optionally substituted with 1 to 3 fluorine atoms.
Also preferred are compounds of general formula (I), wherein
R² represents methyl, difluormethyl, or trifluoromethyl.
   Also preferred are compounds of general formula (I), wherein
X is N or CR^{c}; wherein
R^{c} represents H, F, Cl, CN, methyl, or trifluoromethyl.
   Also preferred are compounds of general formula (I), wherein
X is CR^{c}.
   Also preferred are compounds of general formula (I), wherein
R^{c} represents H, F, Cl, or methyl.
   Also preferred are compounds of general formula (I), wherein
R^{c} represents H, F, or Cl, in particular H or F.
   Also preferred are compounds of general formula (I), wherein
R^{c} represents H or F, in particular H.
   Also preferred are compounds of general formula (I), wherein
R^{c} represents H or F, in particular F.
   Also preferred are compounds of general formula (I), wherein
R³ represents H.
   Also preferred are compounds of general formula (I), wherein
R⁴ represents H.
   Also preferred are compounds of general formula (I), wherein
R³ and R⁴ represent H, and
wherein R³ and R⁴ are attached to the 1,2-cyclopropylamide in cis or trans configuration.
   Also preferred are compounds of general formula (I), wherein
R³ and R⁴ represent hydrogen,
   wherein R³ and R⁴ are attached to the 1,2-cyclopropylamide in trans configuration.
   A preferred embodiment of the invention is compounds of general formula (I), wherein
R⁵ represents
   phenyl,
   —(CH₂)p-(C₅-C₇-cycloalkyl), wherein p is 0 or 1, in particular 1,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 nitrogen atoms, or
   bicyclic 8- to 10-membered heteroaryl containing 1, 2 or 3 heteroatoms or heteroatom-containing groups independently selected from NH, N, O, S, SO and SO₂,
wherein R⁵ is optionally substituted as defined in formula (I).
   In accordance with a further aspect, the present invention covers compounds of general formula (I), wherein
p represents 0.
   Another preferred embodiment of the invention is compounds of general formula (I),
wherein
R⁵ represents
   phenyl,
   —(CH₂)ₚ-(C₅-C₇-cycloalkyl), wherein p represents 0 or 1, in particular 0,
   6-membered heteroaryl containing 1 or 2 nitrogen atoms, or
   bicyclic 9-membered heteroaryl containing 1, 2 or 3 heteroatoms or heteroatom-containing groups independently selected from NH, N, O, S, SO and SO₂,
   wherein said R⁵ is optionally substituted at one or more carbon atoms with 1 or 2 substituents which are the same or different and selected from the group consisting of C₁-C₅-alkyl, —OC₁—C₅-alkyl, halogen, OH and CN, wherein said C₁-C₅-alkyl and —OC₁—C₅-alkyl independently are optionally substituted with one or more substituents independently selected from the group consisting of OH, OR² and F, and
   wherein independently, if said R⁵ represents bicyclic 9-membered heteroaryl, each ring nitrogen atom, if present, of said R⁵ is optionally substituted with C₁-C₅-alkyl, which is optionally substituted with OH, OR² or 1 to 5 fluorine atoms, and
R² has the same meaning as defined herein for general formula (I).
   In another preferred embodiment the invention relates to compounds of formula (I),
wherein
R⁵ represents
   phenyl, or
   cyclohexyl,
   wherein R⁵ is optionally substituted with 1 or 2 substituents which are the same or different and selected from the group consisting of C₁-C₅-alkyl, —OC₁—C₅-alkyl, halogen and CN, wherein said C₁-C₅-alkyl and —OC₁—C₅-alkyl independently are optionally substituted with OH, OR² or 1 to 5 fluorine atoms, and
   R² has the same meaning as defined herein for general formula (I)
   Another preferred embodiment of the invention relates to compounds of general formula (I),
wherein
R⁵ represents phenyl optionally substituted with 1 or 2 substituents which are the same or different and selected from the group consisting of C₁-C₅-alkyl, —OC₁—C₅-alkyl, halogen and CN, wherein said C₁-C₅-alkyl and —OC₁—C₅-alkyl independently are optionally substituted with OH, OR² or 1 to 5 fluorine atoms, and
R² has the same meaning as defined herein for general formula (I).
   Another preferred embodiment of the invention relates to compounds of general formula (I), wherein
R⁵ represents phenyl optionally substituted with 1 or 2 substituents which are the same or different and selected from the group consisting of C₁-C₅-alkyl, —OC₁—C₅-alkyl, F and Cl, wherein said C₁-C₅-alkyl and —OC₁—C₅-alkyl independently are optionally substituted with OH, OR² or 1 to 5 fluorine atoms, and
R² has the same meaning as defined herein for general formula (I).
   Also preferred are compounds of general formula (I), wherein
R⁵ represents phenyl substituted with 1 or 2 substituents which are the same or different and selected from the group consisting of C₁-C₅-alkyl, —OC₁—C₅-alkyl, F and Cl,
   wherein the substituent or at least one of said substituents is C₁-C₅-alkyl, OC₁-C₅-alkyl or Cl, said substitutent or said at least one of said substitutents is preferably positioned meta or para to the carbon atom which links the phenyl to the rest of the molecule, and
   wherein said C₁-C₅-alkyl and OC₁-C₅-alkyl independently are optionally substituted with 1 to 5 fluorine atoms.
   Also preferred are compounds of general formula (I), wherein
R⁵ represents phenyl optionally substituted with 1 or 2 substituents which are the same or different and selected from the group consisting of methyl, trifluoromethyl, trifluoromethoxy, F and Cl,
   wherein if the substituent or at least one of said substituents is F, methyl, trifluoromethyl, trifluoromethoxy or Cl, it is preferably positioned meta or para to the carbon atom which links the phenyl to the rest of the molecule.
   Also preferred are compounds of general formula (I), wherein
R⁵ represents phenyl optionally substituted with 1 or 2 substituents which are the same or different and selected from the group consisting of methyl, trifluoromethyl, trifluoromethoxy, F and Cl,
   wherein if the substituent or at least one of said substituents is F, it is preferably positioned ortho to the carbon atom which links the phenyl to the rest of the molecule, and
wherein if the substituent or at least one of said substituents is methyl, trifluoromethyl, trifluoromethoxy or Cl, it is preferably positioned para to the carbon atom which links the phenyl to the rest of the molecule.

In accordance with a further aspect, the present invention covers compounds of general formula (I), wherein
R⁵ represents
   5-membered heteroaryl containing 1 or 2 nitrogen atoms, in particular pyrazolyl,
      wherein each ring nitrogen atom of the 5-membered heteroaryl independently is optionally substituted with C₁-C₅-alkyl, which is optionally substituted with OH, OR² or 1 to 5 fluorine atoms, and
R² has the same meaning as defined herein for general formula (I).
   Also preferred are compounds of general formula (I), wherein
R⁵ represents pyrazolyl, in particular pyrazol-4-yl, wherein each ring nitrogen atom of the pyrazolyl independently is optionally substituted with C₁-C₅-alkyl, which is optionally substituted with OH, OR² or 1 to 5 fluorine atoms, and
R² has the same meaning as defined herein for general formula (I).
   Also preferred are compounds of general formula (I), wherein
R⁵ represents pyrazolyl, in particular pyrazol-4-yl, substituted at the nitrogen atom at position 1 with C₁-C₅-alkyl, which is optionally substituted with OH, OR² or 1 to 5 fluorine atoms, and
R² has the same meaning as defined herein for general formula (I).
   Also preferred are compounds of general formula (I), wherein
R⁵ represents pyrazolyl, in particular pyrazol-4-yl, substituted at the nitrogen atom at position 1 with methyl.
   Another preferred embodiment of the invention relates to compounds of general formula (I), wherein
R^{d} represents H, OH, F, or methyl, in particular H.
   A preferred embodiment of the invention relates to compounds of general formula (I),
wherein
R^{d} represents H or F, in particular H.
   A preferred embodiment of the invention relates to compounds of general formula (I),
wherein
R^{e} represents H, or F, in particular H.

A further preferred embodiment of the invention is compounds of general formula (I),
wherein
A represents tetrazolyl which is attached to the rest of the molecule by the carbon atom;
R¹ represents
   phenyl,
   5- to 7-membered lactam,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N atoms, or
   bicyclic 9- or 10-membered heteroaryl containing 1, 2 or 3 heteroatoms or heteroatom-containing groups independently selected from NH, N, O, S, SO and SO₂,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently, if R¹ represents 5-membered heteroaryl or bicyclic 9- or 10-membered heteroaryl any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I);
R³ and R⁴ represent H;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.
   In accordance with a further aspect, the present invention covers compounds of general formula (I), wherein
A represents tetrazolyl which is attached to the rest of the molecule by the carbon atom;
R¹ represents
   phenyl,
   5- or 6-membered heteroaryl, wherein said 5-membered heteroaryl contains 1 or 2 heteroatoms or heteroatom-containing groups independently selected from the group consisting of N, NH, and O, and wherein said 6-membered heteroaryl contains 1 or 2 N atoms, or
   bicyclic 9-membered heteroaryl containing 1, 2 or 3 heteroatoms or heteroatom-containing groups independently selected from NH, N, O, S, SO and SO₂,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁-C₅-alkyl, —OC₃—C₅-cycloalkyl, halogen or CN, and
   wherein independently, if R¹ represents 5-membered heteroaryl or bicyclic 9-membered heteroaryl any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I);
R³ and R⁴ represent H;
X represents CR^{c} or N, in particular CR^{c};
R^{c} represents H, F, Cl, methyl, trifluoromethyl or CN, in particular H or F;
R^{d} represents H or F, in particular H; and
R^{e} represents H or F, in particular H.
   A further preferred embodiment of the invention is compounds of general formula (I),
wherein
R³ and R⁴ represent H;
R¹ is selected from the group consisting of phenyl, pyridinyl, pyrimidinyl, pyrazolyl, imidazolyl, benzothiophenyl, or pyrrolidinyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently, if R¹ represents pyrazolyl or imidazolyl any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I); and
   wherein R³ and R⁴ are attached to the 1,2-cyclopropylamide in cis or trans configuration, preferably in trans configuration.
   Additionally preferred are compounds of general formula (I), wherein
R³ and R⁴ represent H;
R¹ represents phenyl,
   wherein said phenyl is optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁-C₅-alkyl and —OC₃—C₅-cycloalkyl independently are optionally substituted with one or more substituents independently selected from the group consisting of OH, OR² and F, and
R² has the same meaning as defined herein for general formula (I); and
wherein R³ and R⁴ are attached to the 1,2-cyclopropylamide in cis or trans configuration, preferably in trans configuration.
   Also preferred are compounds of general formula (I), wherein
R³ and R⁴ represent H;
R¹ represents
   phenyl,
   wherein said R¹ is optionally substituted at one or more carbon atoms with 1 or 2 substituents selected from the group consisting of methyl, methoxy, ethoxy, methoxymethyl, trifluoromethoxy, and a halogen, particular Cl; and
wherein R³ and R⁴ are attached to the 1,2-cyclopropylamide in cis or trans configuration, preferably in trans configuration.
   Also preferred are compounds of general formula (I), wherein
R³ and R⁴ represent H;
R¹ represents
   phenyl,
   wherein said R¹ is optionally substituted at two carbon atoms with 1 substituent at each substituted carbon atom, wherein the two substituents are independently selected from the group of combinations consisting of dimethoxy (methoxy and methoxy), ethoxy and methoxy, methoxymethyl and methyl, methyl and methoxy, and methoxy and halogen, in particular Cl;
   and
wherein R³ and R⁴ are attached to the 1,2-cyclopropylamide in cis or trans configuration, preferably in trans configuration.
   Additionally preferred are compounds of general formula (I), wherein
R³ and R⁴ represent H;
R¹ represents a 5-membered heteroaryl containing 1, 2 or 3 heteroatoms or heteroatom-containing groups selected from N, NH, O and S, in particular pyrazolyl or imidazolyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃-C₅-cycloalkyl, halogen or CN, and
   wherein independently, if R¹ represents pyrazolyl or imidazolyl any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I); and
wherein R³ and R⁴ are attached to the 1,2-cyclopropylamide in cis or trans configuration, preferably in trans configuration.

In another preferred embodiment, the invention relates to compounds of general formula (I), wherein
R³ and R⁴ represent H;
R¹ represents a 9-membered bicyclic heteroaryl containing 1, 2 or 3 heteroatoms or heteroatom-containing groups selected from NH, N, O, S, SO and SO₂, in particular benzopyrazolyl, benzimidazolyl, benzothiophenyl, benzothiazolyl, benzoxadiazolyl, benzothiadiazolyl, thiazolopyridinyl, imidazopyridinyl, oxazolopyridinyl, furopyridinyl, benzofuranyl or thienopyridynyl, more particular benzothiophenyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
      wherein independently each ring nitrogen atom, if present, of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
      if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I); and
wherein R³ and R⁴ are attached to the 1,2-cyclopropylamide in cis or trans configuration, preferably in trans configuration.
   In another preferred embodiment, the invention relates to compounds of general formula (I), wherein
R³ and R⁴ represent H;
R¹ represents phenyl, pyridinyl, pyrimidinyl, pyrazolyl, imidazolyl, benzothiophenyl, or pyrrolidinyl
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁-C₅-alkyl, —OC₃—C₅-cycloalkyl, halogen or CN, and
   wherein independently, if R^{1a} represents pyrazolyl, imidazolyl, or pyrrolidinyl, any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I); and
wherein R³ and R⁴ are attached to the 1,2-cyclopropylamide in cis or trans configuration, preferably in trans configuration.
   In accordance with a first aspect, the present invention covers compounds of general formula (I), wherein
A represents tetrazolyl which is attached to the rest of the molecule by the carbon atom;
R³ and R⁴ represent H;
R^{e} represents H or F, in particular H;
X represents CR^{c} or N, in particular CR^{c};
R^{c} represents H, F, Cl or methyl, in particular H or F; and
R⁵ represents
   phenyl,
   —(CH₂)p-(C₅-C₇-cycloalkyl), wherein p is 0 or 1, in particular 1,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 nitrogen atoms, or
   bicyclic 8- to 10-membered heteroaryl containing 1, 2 or 3 heteroatoms or heteroatom-containing groups independently selected from NH, N, O, S, SO and SO₂; and
wherein R⁵ is optionally substituted as defined in formula (I); and
wherein R³ and R⁴ are attached to the 1,2-cyclopropylamide in cis or trans configuration, preferably in trans configuration.

In another preferred embodiment, the invention relates to compounds of general formula (I), wherein
A represents tetrazolyl which is attached to the rest of the molecule by the carbon atom;
R³ and R⁴ represent H;
R^{e} represents H or F, in particular H;
X represents CR^{c} or N, in particular CR^{c};
R^{c} represents H, F, Cl or methyl, in particular H or F;
R⁵ represents
   phenyl,
   —(CH₂)p-(C₅-C₇-cycloalkyl), wherein p represents 0 or 1, in particular 0, or
   5-membered heteroaryl, wherein said 5-membered heteroaryl contains 1, 2 or 3 heteroatoms or heteroatom-containing groups independently selected from the group consisting of S, N, NH, and O,
   wherein said R⁵ is optionally substituted at one or more carbon atoms with 1 or 2 substituents which are the same or different and selected from the group consisting of C₁-C₅-alkyl, —OC₁—C₅-alkyl, halogen, OH and CN, wherein said C₁-C₅-alkyl and —OC₁—C₅-alkyl independently are optionally substituted with (a) substituent(s) independently selected from the group consisting of OH, OR², and 1 to 5 fluorine atoms, and
   wherein independently, if said R⁵ represents 5-membered heteroaryl, each ring nitrogen atom of said 5-membered heteroaryl is optionally substituted with C₁-C₅-alkyl, which is optionally substituted with OH, OR² or 1 to 5 fluorine atoms, and
R² has the same meaning as defined herein for general formula (I); and
wherein R³ and R⁴ are attached to the 1,2-cyclopropylamide in cis or trans configuration, preferably in trans configuration.

In another preferred embodiment, the invention relates to compounds of general formula (I), wherein
A represents tetrazolyl which is attached to the rest of the molecule by the carbon atom;
R³ and R⁴ represent H;
R^{e} represents H or F, in particular H;
X represents CR^{c} or N, in particular CR^{c};
R^{c} represents H, fluoro, chloro or methyl, in particular H or fluoro; and
R⁵ represents
   phenyl,
   cyclohexyl, or
   pyrazolyl,
      wherein R⁵ is optionally substituted at one or more carbon atoms with 1 or 2 substituents which are the same or different and selected from the group consisting of C₁-C₅-alkyl, OC₁-C₅-alkyl, F, and Cl,
      wherein each ring nitrogen atom, if present, of said R⁵ independently is optionally substituted with C₁-C₅-alkyl, which is optionally substituted with OH, OR² or 1 to 5 fluorine atoms; and
wherein R³ and R⁴ are attached to the 1,2-cyclopropylamide in cis or trans configuration, preferably in trans configuration.
   In another preferred embodiment, the invention relates to compounds of general formula (I), wherein
A represents tetrazolyl which is attached to the rest of the molecule by the carbon atom;
R³ and R⁴ represent H;
R^{d} represents H, OH, F, methyl;
R^{e} represents H or F, in particular H;
X represents CR^{c} or N, in particular CR^{c};
R^{c} represents H, F, Cl or methyl, in particular H or F;
R¹ represents
   phenyl,
   5- or 6-membered heteroaryl, wherein said 5-membered heteroaryl contains 1 or 2 heteroatoms or heteroatom-containing groups independently selected from the group consisting of N, NH, and O, and wherein said 6-membered heteroaryl contains 1 or 2 N atoms, or
   bicyclic 9-membered heteroaryl containing 1, 2 or 3 heteroatoms or heteroatom-containing groups independently selected from NH, N, O, S, SO and SO₂,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently, if R¹ represents 5-membered heteroaryl or bicyclic 9-membered heteroaryl any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R⁵ represents
   phenyl,
   —(CH₂)ₚ-(C₅-C₇-cycloalkyl), wherein p represents 0 or 1, in particular 0,
   5-membered heteroaryl, wherein said 5-membered heteroaryl contains 1, 2 or 3 heteroatoms or heteroatom-containing groups independently selected from the group consisting of S, N, NH, and O, or
   Bicyclic 9-membered heteroaryl containing 1, 2 or 3 heteroatoms or heteroatom-containing groups independently selected from NH, N, O, S, SO and SO₂,
   wherein said R⁵ is optionally substituted at one or more carbon atoms with 1 or 2 substituents which are the same or different and selected from the group consisting of C₁-C₅-alkyl, —OC₁—C₅-alkyl, halogen, OH and CN, wherein said C₁-C₅-alkyl and —OC₁—C₅-alkyl independently are optionally substituted with one or more substituents independently selected from the group consisting of OH, OR² and F;
wherein R² has the same meaning as defined herein for general formula (I); and
wherein independently, if said R⁵ represents 5-membered heteroaryl or bicyclic 9-membered heteroaryl, each ring nitrogen atom, if present, of said R⁵ is optionally substituted with C₁-C₅-alkyl, which is optionally substituted with OH, OR² or 1 to 5 fluorine atoms;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, methyl;
R^{e} represents H or F, in particular H;
X represents CR^{c} or N, in particular CR^{c};
R^{c} represents H, F, Cl or methyl, in particular H or F;
R¹ is selected from the group consisting of phenyl, pyridinyl, pyrimidinyl, pyrazolyl, imidazolyl, benzothiophenyl, and pyrrolidinyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently, if R¹ represents pyrazolyl or imidazolyl any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F; and
R⁵ represents
   phenyl,
   cyclohexyl, or
   pyrazolyl,
   wherein R⁵ is optionally substituted at one or more carbon atoms with 1 or 2 substituents which are the same or different and selected from the group consisting of C₁-C₅-alkyl, OC₁-C₅-alkyl, F and Cl,
   wherein each ring nitrogen atom of said pyrazolyl independently is optionally substituted with C₁-C₅-alkyl, which is optionally substituted with OH, OR² or 1 to 5 fluorine atoms;
wherein R² has the same meaning as defined herein for general formula (I); and
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, methyl;
R^{e} represents H or F, in particular H;
X represents CR^{c} or N, in particular CR^{c};
R^{c} represents H, F, Cl or methyl, in particular H or F;
R¹ represents phenyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁-C₅-alkyl, —OC₃—C₅-cycloalkyl, halogen or CN, and
   if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F; and
R⁵ represents phenyl,
   wherein R⁵ is optionally substituted at one or more carbon atoms with 1 or 2 substituents which are the same or different and selected from the group consisting of C₁-C₅-alkyl, OC₁-C₅-alkyl, F and Cl, and
   wherein said C₁-C₅-alkyl and —OC₁—C₅-alkyl independently are optionally substituted with one or more substituents independently selected from the group consisting of OH, OR² and F;
wherein R² has the same meaning as defined herein for general formula (I); and
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents phenyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F; and
R⁵ represents cyclohexyl,
   wherein R⁵ is optionally substituted at one or more carbon atoms with 1 or 2 substituents which are the same or different and selected from the group consisting of C₁-C₅-alkyl, OC₁-C₅-alkyl, F and Cl, and
   wherein said C₁-C₅-alkyl and —OC₁—C₅-alkyl independently are optionally substituted with one or more substituents independently selected from the group consisting of OH, OR² and F;
wherein R² has the same meaning as defined herein for general formula (I); and
wherein R³ and R⁴ are attached to the 1,2-cyclopropylamide in cis or trans configuration, preferably in trans configuration.

A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents phenyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
R² has the same meaning as defined herein for general formula (I); and
R⁵ represents cyclohexyl,
   wherein R⁵ is unsubstituted or substituted at one carbon atoms with 1 C₁-C₅-alkyl, in particular methyl;
   wherein said C₁-C₅-alkyl, if present, in particular methyl, is positioned para to the carbon atom that links the cyclohexyl to the rest of the molecule;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents phenyl,
   wherein said R¹ is substituted at one carbon atom with a C₁-C₅-alkyl substituted with a —OC₁-C₅-alkyl, in particular methoxymethyl, or
   wherein said R¹ is substituted at one carbon atom with —OC₁-C₅-alkyl optionally substituted with one or more F, and wherein said R¹ is optionally substituted at a second carbon atom with a substituent selected from the group consisting of —OC₁-C₅-alkyl, C₁-C₅-alkyl and halogen, in particular methyl and Cl; and
R⁵ represents cyclohexyl,
   wherein R⁵ is unsubstituted or substituted at one carbon atoms with a C₁-C₅-alkyl, in particular methyl;
   wherein said C₁-C₅-alkyl, if present, in particular methyl, is positioned para to the carbon atom that links the cyclohexyl to the rest of the molecule;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents phenyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F; and
R⁵ represents pyrazolyl,
   wherein R⁵ is optionally substituted at one or more carbon atoms with 1 or 2 substituents which are the same or different and selected from the group consisting of C₁-C₅-alkyl, OC₁-C₅-alkyl, F and Cl,
   wherein each ring nitrogen atom of said pyrazolyl independently is optionally substituted with C₁-C₅-alkyl, which is optionally substituted with OH, OR² or 1 to 5 fluorine atoms;
wherein R² has the same meaning as defined herein for general formula (I); and
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ represents H or methyl, in particular H;
R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyridinyl, in particular pyridin-3-yl,
   wherein said R¹ is optionally substituted at one or more carbon atoms with 1 or 2 substituents R^{1a} which are the same or different wherein R^{1a} represents C₁-C₅-alkyl or —OC₁-C₅-alkyl, and
   wherein said C₁-C₅-alkyl and —OC₁-C₅-alkyl independently are unsubstituted or substituted with one or more substituents independently selected from the group consisting of OH, OR² and F; and
R⁵ represents phenyl,
   wherein R⁵ is optionally substituted at one or more carbon atoms with 1 or 2 substituents which are the same or different and selected from the group consisting of C₁-C₅-alkyl, OC₁-C₅-alkyl, F and Cl, and
   wherein said C₁-C₅-alkyl and —OC₁-C₅-alkyl independently are optionally substituted with one or more substituents independently selected from the group consisting of OH, OR² and F;
wherein R² has the same meaning as defined herein for general formula (I); and
wherein R³ and R⁴ are attached to the 1,2-cyclopropylamide in cis or trans configuration, preferably in trans configuration.

A further preferred embodiment of the invention is compounds of general formula (I), wherein
A represents tetrazolyl which is attached to the rest of the molecule by the carbon atom;
R³ represents H or methyl, in particular H;
R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyridinyl, in particular pyridin-3-yl,
   wherein said R¹ is optionally substituted at one carbon atom with a substituent R^{1a} which represents C₁-C₅-alkyl or —OC₁-C₅-alkyl, and
   wherein said C₁-C₅-alkyl is substituted with one or more F, in particular R^{1a} represent a substituent selected from the group consisting of methoxy, ethoxy, isopropoxy, and trifluoromethyl;
R⁵ represents phenyl,
   wherein R⁵ is optionally substituted at one carbon atom with a substitutent selected from the group consisting of C₁-C₅-alkyl, F and Cl,
   in particular R⁵ is unsubstituted or substituted with a substituent selected from the group consisting of methyl, F and Cl;
wherein R³ and R⁴ are attached to the 1,2-cyclopropylamide in cis or trans configuration, preferably in trans configuration.
   A further preferred embodiment of the invention is compounds of general formula (I), wherein

A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyridinyl, in particular pyridin-3-yl,
   wherein said R¹is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN,

if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F;
R⁵ represents pyrazolyl,
   wherein R⁵ is optionally substituted at one or more carbon atoms with 1 or 2 substituents which are the same or different and selected from the group consisting of C₁-C₅-alkyl, OC₁-C₅-alkyl, F and Cl,
   wherein each ring nitrogen atom of said pyrazolyl independently is optionally substituted with C₁-C₅-alkyl, which is optionally substituted with OH, OR₂ or 1 to 5 fluorine atoms;
wherein R² has the same meaning as defined herein for general formula (I); and
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyridinyl, in particular pyridin-3-yl,
   wherein said R¹ is optionally substituted at one carbon atom, in particular at carbon atom at position 6 of pyrdin-3-yl, with a substituent R^{1a}, wherein R^{1a} represents —OC₁—C₅-alkyl, in particular R^{1a} is ethoxy;
R⁵ represents pyrazolyl, in particular pyrazol-4-yl,
   wherein R⁵ is optionally substituted at one ring nitrogen atom of said pyrazolyl, in particular at the nitrogen at position 1 of said pyrazol-4-yl, with a C₁-C₅-alkyl, which is optionally substituted with OH, OR² or 1 to 5 fluorine atoms, in particular with unsubstituted methyl, and

R² has the same meaning as defined herein for general formula (I);
wherein R³ and R⁴ are attached to the 1,2-cyclopropylamide in cis or trans configuration, preferably in trans configuration.

A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyridinyl, in particular pyridin-3-yl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F,
R² has the same meaning as defined herein for general formula (I);
R⁵ represents cyclohexyl,
   wherein R⁵ is optionally substituted at one or more carbon atoms with 1 or 2 substituents which are the same or different and selected from the group consisting of C₁-C₅-alkyl, OC₁-C₅-alkyl, F and Cl;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyridinyl, in particular pyridin-3-yl,
   wherein said R¹ is optionally substituted at one carbon atom with a substituent R^{1a} which represents C₁-C₅-alkyl, or —OC₁—C₅-alkyl,
   if R^{1a} represents C₁-C₅-alkyl, said C₁-C₅-alkyl is optionally substituted with 1 to 3 F,
   wherein particular said R^{1a} is selected from the group consisting of trifluoromethyl, methoxyl, and ethoxyl;
R⁵ represents cyclohexyl,
   wherein R⁵ is unsubstituted or substituted at one carbon atom with a C₁-C₅-alkyl, in particular methyl;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyrimidinyl,
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F; and
R⁵ represents phenyl,
   wherein R⁵ is optionally substituted at one or more carbon atoms with 1 or 2 substituents which are the same or different and selected from the group consisting of C₁-C₅-alkyl, OC₁-C₅-alkyl, F and Cl, and
   wherein said C₁-C₅-alkyl and —OC₁—C₅-alkyl independently are optionally substituted with one or more substituents independently selected from the group consisting of OH, OR² and F;
wherein R² has the same meaning as defined herein for general formula (I); and
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyrimidinyl, in particular pyrimidin-5-yl, optionally substituted at one or more carbon atoms with 1 or 2 substituents R^{1a} being —OC₁-C₅-alkyl,
   wherein said —OC₁—C₅-alkyl is preferably positioned para to the carbon atom which links the pyrimidinyl, in particular pyrimidin-5-yl, to the rest of the molecule,
   wherein said —OC₁—C₅-alkyl is optionally substituted with one or more substituents independently selected from the group consisting of OH, OR² and F, and
   R² has the same meaning as defined herein for general formula (I); and in particular R¹ represents ethoxypyrmidinyl, more particular 2-ethoxypyrimidin-5yl; and
R⁵ represents phenyl,
   wherein R⁵ is optionally substituted with Cl, in particular at the carbon atom positioned para to the carbon atom which links the phenyl to the rest of the molecule, in particular R⁵ is 4-chlorophenyl;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyrazolyl, in particular pyrazol-1-yl or pyrazol-4-yl, optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently any ring nitrogen atom of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, -(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F; and
R⁵ represents phenyl optionally substituted with 1 or 2 substituents which are the same or different and selected from the group consisting of C₁-C₅-alkyl, —OC₁—C₅-alkyl, halogen and CN, wherein said C₁-C₅-alkyl and —OC₁—C₅-alkyl independently are optionally substituted with OH, OR² or 1 to 5 fluorine atoms;
wherein R² has the same meaning as defined herein for general formula (I); and
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;

R¹ represents pyrazolyl, in particular pyrazol-1-yl or pyrazol-4-yl, optionally substituted at one or more carbon atoms with 1 or 2 substituents R^{1a} which are the same or different wherein R^{1a} represents C₁-C₅-alkyl, C₃-C₅-cycloalkyl or —(C₁-C₃-alkyl)—(C₃-C₅-cycloalkyl),
wherein R^{1a} is optionally substituted with one or more substituents independently selected from the group consisting of OH, OR² and F; and
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or Cl, said substitutent or said at least one of said substitutents is preferably positioned meta or para to the carbon atom which links the phenyl to the rest of the molecule, and
   wherein said C₁-C₅-alkyl and OC₁-C₅-alkyl independently are optionally substituted with 1 to 5 fluorine atoms;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyrazol-1-yl optionally substituted at any carbon atom with a substituent selected from the group consisting of methyl, butyl, in particular tert-butyl, and trifluoromethyl; and
R⁵ represents phenyl optionally substituted with 1 or 2 substituents which are the same or different and selected from the group consisting of methyl, trifluoromethoxy, F and Cl,
   wherein if the substituent or at least one of said substituents is F, methyl, trifluoromethyl, trifluoromethoxy or Cl, it is preferably positioned meta or para to the carbon atom which links the phenyl to the rest of the molecule;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyrazol-1-yl optionally substituted at the carbon atom at position 3 or 4 with a substituent selected from the group consisting of tert-butyl and trifluoromethyl.
R⁵ represents phenyl optionally substituted with 1 or 2 substituents which are the same or different and selected from the group consisting of methyl, trifluoromethoxy, F and Cl,
   wherein if the substituent or at least one of said substituents is F, methyl, trifluoromethyl, trifluoromethoxy or Cl, it is preferably positioned meta or para to the carbon atom which links the phenyl to the rest of the molecule;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents pyrazol-4-yl optionally substituted at any ring nitrogen atom of said R¹ with 1 substituent R^{1b}, wherein R^{1b} represents C₃-C₅-cycloalkyl, in particular cyclobutyl;and preferably, said R^{1b} is at the ring nitrogen at position 1 of the pyrazol-4-yl; and
R⁵ represents phenyl optionally substituted with 1 or 2 substituents which are the same or different and selected from the group consisting of trifluoromethy, and F,
   wherein if the substituent or at least one of said substituents is F, it is preferably positioned ortho to the carbon atom which links the phenyl to the rest of the molecule, and
   wherein if the substituent or at least one of said substituents is trifluoromethyl, it is preferably positioned para to the carbon atom which links the phenyl to the rest of the molecule;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H;
R^{e} represents H;
X represents CR^{c};
R^{c} represents H;
R¹ represents pyrazol-4-yl substituted with cyclobutyl at the ring nitrogen atom at position 1; and
R⁵ represents phenyl substituted with trifluoromethyl and F,
   wherein F is positioned ortho to the carbon atom which links the phenyl to the rest of the molecule, and
   wherein the trifluoromethyl is preferably positioned para to the carbon atom which links the phenyl to the rest of the molecule;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents a 9-membered bicyclic heteroaryl containing 1, 2 or 3 heteroatoms or heteroatom-containing groups selected from NH, N, O, S, SO and SO₂, in particular R¹ represents benzothiophenyl;
   wherein said R¹ is optionally substituted at one or more carbon atoms with 1 or 2 substituents R^{1a} which are the same or different wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   wherein independently each ring nitrogen atom, if present, of said R¹ is optionally substituted with 1 substituent R^{1b} wherein R^{1b} represents C₁-C₅-alkyl, —(C₁-C₃-alkyl)—(C₃-C₅-cycloalkyl) or C₃-C₅-cycloalkyl, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F; and R² has the same meaning as defined herein for general formula (I);
R⁵ represents phenyl substituted with 1 or 2 substituents which are the same or different and selected from the group consisting of C₁-C₅-alkyl, OC₁-C₅-alkyl, F and Cl,
   wherein the substituent or at least one of said substituents is C₁-C₅-alkyl, OC₁-C₅-alkyl or Cl, and
   wherein said C₁-C₅-alkyl and OC₁-C₅-alkyl independently are optionally substituted with 1 to 5 fluorine atoms;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents benzothiophenyl; and
R⁵ represents phenyl optionally substituted with 1 C₁-C₅-alkyl, in particular methyl,
   wherein said C₁-C₅-alkyl is preferably positioned meta to the carbon atom which links the phenyl to the rest of the molecule;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents a 5- to 7-membered lactam,
   wherein said R¹ is optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
wherein the ring nitrogen atom of said 5- to 7-membered lactam is optionally substituted with 1 substituent R^{1b}, wherein R^{1b} represents C₁-C₅-alkyl, C₃-C₅-cycloalkyl, or —(C₁-C₃-alkyl)—(C₃-C₅-cycloalkyl), and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F; and
R⁵ represents phenyl optionally substituted with 1 or 2 substituents which are the same or different and selected from the group consisting of C₁-C₅-alkyl, —OC₁—C₅-alkyl, halogen and CN, wherein said C₁-C₅-alkyl and —OC₁—C₅-alkyl independently are optionally substituted with OH, OR² or 1 to 5 fluorine atoms;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents 5-membered lactam attached to the rest of the molecule through its ring nitrogen atom,
   wherein said R¹ is optionally substituted at one or more carbon atoms with 1 or 2 substituents R^{1a} which are the same or different, wherein R^{1a} represents C₁-C₅-alkyl, C₃-C₅-cycloalkyl, —(C₁-C₃-alkyl)—(C₃-C₅-cycloalkyl), —OC₁—C₅-alkyl, —OC₃—C₅-cycloalkyl, halogen or CN, and
   if R^{1a} represents C₁-C₅-alkyl, C₃-C₅-cycloalkyl, —(C₁-C₃-alkyl)—(C₃-C₅-cycloalkyl), —OC₁—C₅-alkyl or —OC₃—C₅-cycloalkyl, said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
   R² has the same meaning as defined herein for general formula (I); and
R⁵ represents phenyl optionally substituted with 1 or 2 substituents which are the same or different and selected from the group consisting of C₁-C₅-alkyl, —OC₁—C₅-alkyl, F, Cl and CN, wherein said C₁-C₅-alkyl and —OC₁—C₅-alkyl independently are optionally substituted with OH, OR² or 1 to 5 fluorine atoms;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents 5-membered lactam,
   wherein said R¹ is optionally substituted at one carbon atom with a substituent R^{1a}, wherein R^{1a} represents C₁-C₅-alkyl, C₃-C₅-cycloalkyl, —(C₁-C₃-alkyl)—(C₃-C₅-cycloalkyl), —OC₁-C₅-alkyl, —OC₃—C₅-cycloalkyl, halogen or CN,
   wherein the ring nitrogen atom of said 5-membered lactam links the lactam to the rest of the molecule, and
   wherein said C₁-C₅-alkyl, C₃-C₅-cycloalkyl, —(C₁-C₃-alkyl)—(C₃-C₅-cycloalkyl), —OC₁-C₃-alkyl and —OC₃—C₅-cycloalkyl is optionally substituted with one or more substituents independently selected from the group consisting of OH, OR² and F, and
   R² has the same meaning as defined herein for general formula (I); and
R⁵ represents phenyl optionally substituted with a halogen, in particular Cl;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents 5-membered lactam,
   wherein said R¹ is optionally substituted at one carbon atom with a substituent R^{1a}, wherein R^{1a} represents C₁-C₅-alkyl, in particular ethyl,
   wherein the ring nitrogen atom of said 5-membered lactam links the lactam to the rest of the molecule; and
R⁵ represents phenyl optionally substituted with a halogen, in particular Cl, in particular said halogen is positioned para to the carbon atom that links the phenyl to the rest of the molecule;
wherein R³ and R⁴ are attached to the 1,2-cyclopropylamide in cis or trans configuration, preferably in trans configuration.
   A further preferred embodiment of the invention is compounds of general formula (I), wherein
A represents tetrazolyl which is attached to the rest of the molecule by the carbon atom;
R³ and R⁴ represent H;
R^{d} represents H, OH, F, or methyl, in particular H;
R^{e} represents H or F, in particular H;
X represents CR^{c} or N, in particular CR^{c};
R^{c} represents H, F, Cl or methyl, in particular H or F;
R¹ represents oxopyrrolidinyl, in particular 2-oxopyrrolidin-1-yl,
   wherein said R¹ is optionally substituted at one carbon atom with a C₁-C₅-alkyl, in particular ethyl,
   in particular R¹ represents 3-ethyl-2-oxopyrrolidin-1-yl; and
R⁵ represents phenyl optionally substituted with a halogen, in particular Cl, in particular said halogen is positioned para to the carbon atom that links the phenyl to the rest of the molecule;
wherein R³ and R⁴ are attached to the 1,2-cyclopropylamide in cis or trans configuration, preferably in trans configuration.
   Particular preferred compounds are selected from the group consisting of
trans-N-[3′,4′-dimethoxy-2-(1H-tetrazol-5-yl)biphenyl-4-yl]-2-phenylcyclopropanecarboxamide;
trans-N-[3′,4′-dimethoxy-2-(1H-tetrazol-5-yl)biphenyl-4-yl]-2-(1-methyl-1H-pyrazol-4-yl)cyclopropanecarboxamide;
trans-N-[3′,4′-dimethoxy-2-(1H-tetrazol-5-yl)biphenyl-4-yl]-2-(2-fluorophenyl)cyclopropanecarboxamide;
trans-N-[3′,4′-dimethoxy-2-(1H-tetrazol-5-yl)biphenyl-4-yl]-2-(2-methylphenyl)cyclopropanecarboxamide;
trans-N-[3′,4′-dimethoxy-2-(1H-tetrazol-5-yl)biphenyl-4-yl]-2-(2-ethylphenyl)cyclopropanecarboxamide;
trans-N-[3′,4′-dimethoxy-2-(1H-tetrazol-5-yl)biphenyl-4-yl]-2-[2-(trifluoromethoxy)phenyl]cyclopropanecarboxamide;
trans-N-[3′,4′-dimethoxy-2-(1H-tetrazol-5-yl)biphenyl-4-yl]-2-(3-methylphenyl)cyclopropanecarboxamide;
trans-2-(3-chlorophenyl)-N-[3′,4′-dimethoxy-2-(1H-tetrazol-5-yl)biphenyl-4-yl]cyclopropanecarboxamide;
trans-2-(4-chlorophenyl)-N-[3′,4′-dimethoxy-2-(1H-tetrazol-5-yl)biphenyl-4-yl]cyclopropanecarboxamide;
trans-N-[3′,4′-dimethoxy-2-(1H-tetrazol-5-yl)biphenyl-4-yl]-2-(4-methylphenyl)cyclopropanecarboxamide;
trans-N-[3′,4′-dimethoxy-2-(1H-tetrazol-5-yl)biphenyl-4-yl]-2-(4-fluorophenyl)cyclopropanecarboxamide;
trans-N-[3′,4′-dimethoxy-2-(1H-tetrazol-5-yl)biphenyl-4-yl]-2-(4-ethylphenyl)cyclopropanecarboxamide;
trans-N-[3′,4′-dimethoxy-2-(1H-tetrazol-5-yl)biphenyl-4-yl]-2-(4-methoxyphenyl)cyclopropanecarboxamide;
trans-2-cyclohexyl-N-[3′,4′-dimethoxy-2-(1H-tetrazol-5-yl)biphenyl-4-yl]cyclopropanecarboxamide;
N[3′,4′-dimethoxy-2-(1H-tetrazol-5-yl)biphenyl-4-yl]-2-(4-methylcyclohexyl)cyclopropanecarboxamide;
trans-N-[4-(6-ethoxypyridin-3-yl)-3-(1H-tetrazol-5-yl)phenyl]-2-(4-fluorophenyl)cyclopropanecarboxamide;
trans-2-(4-chlorophenyl)-N-[4-(6-ethoxypyridin-3-yl)-3-(1H-tetrazol-5-yl)phenyl]cyclopropanecarboxamide;
trans-N-[4-(6-ethoxypyridin-3-yl)-3-(1H-tetrazol-5-yl)phenyl]-2-(3-methylphenyl)cyclopropanecarboxamide;
trans-2-(3-chlorophenyl)-N-[4-(6-ethoxypyridin-3-yl)-3-(1H-tetrazol-5-yl)phenyl]cyclopropanecarboxamide;
trans-2-cyclohexyl-N-[4-(6-ethoxypyridin-3-yl)-3-(1H-tetrazol-5-yl)phenyl]cyclopropanecarboxamide;
trans-N-[4-(6-ethoxypyridin-3-yl)-3-(1H-tetrazol-5-yl)phenyl]-1-methyl-2-phenylcyclopropanecarboxamide;
trans-2-(4-chlorophenyl)-N-[4-(5-ethoxypyridin-3-yl)-3-(1H-tetrazol-5-yl)phenyl]cyclopropanecarboxamide;
trans-N-[4-(5-ethoxypyridin-3-yl)-3-(1H-tetrazol-5-yl)phenyl]-2-(3-methylphenyl)cyclopropanecarboxamide;
trans-2-cyclohexyl-N-[4-(5-methoxypyridin-3-yl)-3-(1H-tetrazol-5-yl)phenyl]cyclopropanecarboxamide;
trans-N-[4-(5-methoxypyridin-3-yl)-3-(1H-tetrazol-5-yl)phenyl]-2-(3-methylphenyl)cyclopropanecarboxamide;
trans-2-(4-chlorophenyl)-N-[4-(5-methoxypyridin-3-yl)-3-(1H-tetrazol-5-yl)phenyl]cyclopropanecarboxamide;
trans-2-(4-chlorophenyl)-N-[4-(2-ethoxypyrimidin-5-yl)-3-(1H-tetrazol-5-yl)phenyl]cyclopropanecarboxamide;
trans-2-phenyl-N-{3-(1H-tetrazol-5-yl)-4-[(trifluoromethyl)pyridin-3-yl]phenyl}cyclopropanecarboxamide;
trans-1-methyl-2-phenyl-N-{3-(1-tetrazol-5-yl)-4-[6-(trifluoromethyl)pyridin-3-yl]phenyl}cyclopropanecarboxamide;
trans-2-cyclohexyl-N-{3-(1H-tetrazol-5-yl)-4-[6-(trifluoromethyl)pyridin-3-yl]phenyl}cyclopropanecarboxamide;
trans-2-cyclohexyl-N-[4′-ethoxy-3′-methoxy-2-(1H-tetrazol-5-yl)biphenyl-4-yl]cyclopropanecarboxamide;

trans-2-(4-chlorophenyl)-N-[4′-(methoxymethyl)-3′-methyl-2-(1H-tetrazol-5-yl)biphenyl-4-yl]cyclopropanecarboxamide;
trans-2-cyclohexyl-N-[4′-(methoxymethyl)-3′-methyl-2-(1H-tetrazol-5-yl)biphenyl-4-yl]cyclopropanecarboxamide;
trans-2-(4-chlorophenyl)-N-[4′-methoxy-3′-methyl-2-(1H-tetrazol-5-yl)biphenyl-4-yl]cyclopropanecarboxamide;
trans-2-cyclohexyl-N-[4′-methoxy-3′-methyl-2-(1H-tetrazol-5-yl)biphenyl-4-yl]cyclopropanecarboxamide;
trans-N-[4′-methoxy-2-(1H-tetrazol-5-yl)biphenyl-4-yl]-2-(4-methylphenyl)cyclopropanecarboxamide;
trans-2-cyclohexyl-N-[4′-methoxy-2-(1H-tetrazol-5-yl)biphenyl-4-yl]cyclopropanecarboxamide;
trans-2-(4-chlorophenyl)-N-[4′-(methoxymethyl)-2-(1H-tetrazol-5-yl)biphenyl-4-yl]cyclopropanecarboxamide;
trans-N-[3′-chloro-4′-methoxy-2-(1H-tetrazol-5-yl)biphenyl-4-yl]-2-(4-chlorophenyl)cyclopropanecarboxamide;
trans-N-[3′-chloro-4′-methoxy-2-(1H-tetrazol-5-yl)biphenyl-4-yl]-2-cyclohexylcyclopropanecarboxamide;
trans-2-(4-chlorophenyl)-N-[2-(1H-tetrazol-5-yl)-4′-(trifluoromethoxy)biphenyl-4-yl]cyclopropanecarboxamide;
trans-2-cyclohexyl-N-[2-(1H-tetrazol-5-yl)-4′-(trifluoromethoxy)biphenyl-4-yl]cyclopropanecarboxamide;
trans-2-phenyl-N-{3-(1H-tetrazol-5-yl)-4-[4-(trifluoromethyl)-1H-pyrazol-1-yl]phenyl}cyclopropanecarboxamide;
trans-N-[4-(4-tert-butyl-1H-pyrazol-1-yl)-3-(1H-tetrazol-5-yl)phenyl]-2-(4-chlorophenyl)cyclopropanecarboxamide;
trans-N-[4-(3-tert-butyl-1H-pyrazol-1-yl)-3-(1H-tetrazol-5-yl)phenyl]-2-(4-chlorophenyl)cyclopropanecarboxamide;
trans-2-(4-chlorophenyl)-N-{3-(1H-tetrazol-5-yl)-4-[4-(trifluoromethyl)-1H-pyrazol-1-yl]phenyl}cyclopropanecarboxamide;
trans-2-(4-chlorophenyl)-N-{3-(1H-tetrazol-5-yl)-4-[3-(trifluoromethyl)-1H-pyrazol-1-yl]phenyl}cyclopropanecarboxamide;
trans-N-[4-(4-tert-butyl-1H-imidazol-1-yl)-3-(1H-tetrazol-5-yl)phenyl]-2-(4-chlorophenyl)cyclopropanecarboxamide;
cis-2-(4-chlorophenyl)-N-[4-(6-ethoxypyridin-3-yl)-2-hydroxy-5-(1H-tetrazol-5-yl)phenyl]cyclopropanecarboxamide;
trans-2-(4-chlorophenyl)-N-[4-(6-ethoxypyridin-3-yl)-2-hydroxy-5-(1H-tetrazol-5-yl)phenyl]cyclopropanecarboxamide;
trans-N-[4-(6-ethoxypyridin-3-yl)-2-hydroxy-5-(1H-tetrazol-5-yl)phenyl]2-(3-methylphenyl)cyclopropanecarboxamide;
trans-N-{2-hydroxy-5-(1H-tetrazol-5-yl)-4-[6-(trifluoromethyl)pyridin-3-yl]phenyl}-2-(3-methylphenyl)cyclopropanecarboxamide;
trans-N-[5-fluoro-3′,4′-dimethoxy-2-(1H-tetrazol-5-yl)biphenyl-4-yl]-2-(3-methylphenyl)cyclopropanecarboxamide;
trans-2-(4-chlorophenyl)-N-[5-fluoro-3′,4′-dimethoxy-2-(1H-tetrazol-5-yl)biphenyl-4-yl]cyclopropanecarboxamide;
trans-N-[5-fluoro-4′-methoxy-2-(1H-tetrazol-5-yl)biphenyl-4-yl]-2-(3-methylphenyl)cyclopropanecarboxamide;
trans-N-[5-fluoro-4′-methoxy-3′-methyl-2-(1H-tetrazol-5-yl)biphenyl-4-yl]-2-(3-methylphenyl)cyclopropanecarboxamide;
trans-2-(4-chlorophenyl)-N-[5-fluoro-4′-methoxy-3′-methyl-2-(1H-tetrazol-5-yl)biphenyl-4-yl]cyclopropanecarboxamide;
trans-2-(4-chlorophenyl)-N-[5-fluoro-4′-methoxy-2-(1H-tetrazol-5-yl)biphenyl-4-yl]cyclopropanecarboxamide;
trans-N-[2-fluoro-3′,4′-dimethoxy-6-(1H-tetrazol-5-yl)biphenyl-4-yl]-2-(3-methylphenyl)cyclopropanecarboxamide;
trans-2-cyclohexyl-N-[2-fluoro-3′,4′-dimethoxy-6-(1H-tetrazol-5-yl)biphenyl-4-yl]cyclopropanecarboxamide;
trans-2-(4-chlorophenyl)-N-[4-(6-ethoxypyridin-3-yl)-3-fluoro-5-(1H-tetrazol-5-yl)phenyl]cyclopropanecarboxamide;
trans-2-(4-chlorophenyl)-N-{3-fluoro-5-(1H-tetrazol-5-yl)-4-[6-(trifluoromethyl)pyridin-3-yl]phenyl}cyclopropanecarboxamide;
trans-N-[4-(6-ethoxypyridin-3-yl)-2-methyl-5-(1H-tetrazol-5-yl)phenyl]-2-(3-methylphenyl)cyclopropanecarboxamide;
trans-2-(4-chlorophenyl)-N-[4-(6-ethoxypyridin-3-yl)-2-methyl-5-(1H-tetrazol-5-yl)phenyl]cyclopropanecarboxamide;
trans-2-cyclohexyl-N-[4-(6-ethoxypyridin-3-yl)-2-methyl-5-(1H-tetrazol-5-yl)phenyl]cyclopropanecarboxamide;
trans-2-(4-chlorophenyl)-N-[3′,4′-dimethoxy-5-methyl-2-(1H-tetrazol-5-yl)biphenyl-4-yl]cyclopropanecarboxamide;
trans-2-cyclohexyl-N-[3′,4′-dimethoxy-5-methyl-2-(1H-tetrazol-5-yl)biphenyl-4-yl]cyclopropanecarboxamide;
trans-N-[4-(5-methoxypyridin-3-yl)-2-methyl-5-(1H-tetrazol-5-yl)phenyl]-2-(3-methylphenyl)cyclopropanecarboxamide;
trans-2-cyclohexyl-N-[4-(5-methoxypyridin-3-yl)-2-methyl-5-(1H-tetrazol-5-yl)phenyl]cyclopropanecarboxamide;
trans-2-(3-methylphenyl)-N-{2-methyl-5-(1H-tetrazol-5-yl)-4-[6-(trifluoromethyl)pyridin-3-yl]phenyl}cyclopropanecarboxamide;
trans-2-cyclohexyl-N-{2-methyl-5-(1H-tetrazol-5-yl)-4-[6-(trifluoromethyl)pyridin-3-yl]phenyl}cyclopropanecarboxamide;
trans-N-[4′-ethoxy-3′-methoxy-5-methyl-2-(1H-tetrazol-5-yl)biphenyl-4-yl]-2-(3-methylphenyl)cyclopropanecarboxamide;
trans-2-cyclohexyl-N-[4′-ethoxy-3′-methoxy-5-methyl-2-(1H-tetrazol-5-yl)biphenyl-4-yl]cyclopropanecarboxamide;
trans-2-cyclohexyl-N-[4′-methoxy-3′,5-dimethyl-2-(1H-tetrazol-5-yl)biphenyl-4-yl]cyclopropanecarboxamide;
trans-2-(4-chlorophenyl)-N-{4-[(3R)-3-ethyl-2-oxopyrrolidin-1-yl]-3-(1H-tetrazol-5-yl)phenyl}cyclopropanecarboxamide;
trans-N-[4-(1-benzothiophen-2-yl)-3-(1H-tetrazol-5-yl)phenyl]-2-(3-methylphenyl)cyclopropanecarboxamide;
trans-N-[4′-ethoxy-3′-methoxy-2-(1H-tetrazol-5-yl)biphenyl-4-yl]-2-(3-methylphenyl)cyclopropanecarboxamide;
trans-N-[4-(6-isopropoxypyridin-3-yl)-3-(1H-tetrazol-5-yl)phenyl]-2-(3-methylphenyl)cyclopropanecarboxamide;
trans-2-(4-chlorophenyl)-N-3-(1H-tetrazol-5-yl)-4-[6-(trifluoromethyl)pyridin-3-yl]phenyl}cyclopropanecarboxamide;
trans-2-(4-chlorophenyl)-N-[4-(1-methyl-1H-pyrazol-4-yl)-3-(1H-tetrazol-5-yl)phenyl]cyclopropanecarboxamide;
trans-2-(4-chlorophenyl)-N-[2-hydroxy-5-(1H-tetrazol-5-yl)-4-[6-(trifluoromethyl)-3-pyridyl]phenyl]cyclopropanecarboxamide;
(+)-trans-N-[4-(4-tert-butyl-1H-imidazol-1-yl)-3-(1H-tetrazol-5-yl)phenyl]-2-(4-chlorophenyl)cyclopropanecarboxamide; and
(-)-trans-N-[4-(4-tert-butyl-1H-imidazol-1-yl)-3-(1H-tetrazol-5-yl)phenyl]-2-(4-chlorophenyl)cyclopropanecarboxamide;
or an isomer, enantiomer, diastereomer, racemate, hydrate, solvate, or a salt thereof, or a mixture of same.

It is to be understood that the present invention relates also to any combination of the preferred embodiments described above.

As mentioned above, compounds of the present invention effectively inhibit Bradykinin B1 receptor and may therefore be used for the treatment or prophylaxis of diseases which are related to pain and to inflammation.

Additionally, compounds of the present invention reduce the release of inflammation related cytokines like IL-6 and IL-8.

### Pharmaceutical Compositions of the Compounds of the Invention

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprises at least one compound according to the invention, conventionally together with one or more pharmaceutically suitable excipient(s), and to their use according to the present invention.

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations.

For example, the compounds of this invention can be combined with known hormonal therapeutical agents.

In particular, the compounds of the present invention can be administered in combination or as comedication with hormonal contraceptives. Hormonal contraceptives are for example Combined Oral Contraceptives (COCs) or Progestin-Only-Pills (POPs) or hormone-containing devices.

COCs include but are not limited to birth control pills or a birth control method that includes a combination of an estrogen (estradiol) and a progestogen (progestin). The estrogenic part is in most of the COCs ethinyl estradiol. Some COCs contain estradiol or estradiol valerate.

Said COCs contain the progestins norethynodrel, norethindrone, norethindrone acetate, ethynodiol acetate, norgestrel, levonorgestrel, norgestimate, desogestrel, gestodene, drospirenone, dienogest, or nomegestrol acetate.

Birth control pills include for example but are not limited to Yasmin, Yaz, both containing ethinyl estradiol and drospirenone; Microgynon or Miranova containing levonorgestrel and ethinyl estradiol; Marvelon containing ethinyl estradiol and desogestrel; Valette containing ethinyl estradiol and dienogest; Belara and Enriqa containing ethinyl estradiol and chlormadinonacetate; Qlaira containing estradiol valerate and dienogest as active ingredients; and Zoely containing estradiol and normegestrol.

POPs are contraceptive pills that contain only synthetic progestogens (progestins) and do not contain estrogen. They are colloquially known as mini pills.

POPs include but are not limited to Cerazette containing desogestrel; and Micronor containing norethindrone.

Other Progeston-Only forms are intrauterine devices (IUDs), for example Mirena containing levonorgestrel or injectables, for example Depo-Provera containing medroxyprogesterone acetate, or implants, for example Implanon containing etonogestrel.

Other hormone-containing devices with contraceptive effect which are suitable for a combination with the compounds of the present invention are vaginal rings like Nuvaring containing ethinyl estradiol and etonogestrel, or transdermal systems like contraceptive patches, for example Ortho-Evra containing ethinyl estradiol and norelgestromin or Apleek (Lisvy) containing ethinyl estradiol and gestodene.

A preferred embodiment of the present invention is the administration of a compound of general formula (I) in combination with a COC or a POP or other Progestin-Only forms, as well as in combination with vaginal rings or contraceptive patches as mentioned above.

Furthermore, the compounds of the present invention can be combined with therapeutic agents or active ingredients, that are already approved or that are still under development for the treatment and/or prophylaxis of diseases which are related to or mediated by the Bradykinin B1 receptor.

For the treatment and/or prophylaxis of urinary tract diseases, the compounds of the present invention can be administered in combination or as co-medication with any substance that can be applied as therapeutic agent in the following indications:

Urinary tract disease states associated with the bladder outlet obstruction; urinary incontinence conditions such as reduced bladder capacity, increased frequency of micturition, urge incontinence, stress incontinence, or bladder hyperreactivity; benign prostatic hypertrophy; prostatic hyperplasia; prostatitis; detrusor hyperreflexia; overactive bladder and symptoms related to overactive bladder wherein said symptoms are in particular increased urinary frequency, nocturia, urinary urgency or urge incontinence; pelvic hypersensitivity; urethritis; prostatitis; prostatodynia; cystitis, in particular interstitial cystitis; idiopathic bladder hypersensitivity.

For the treatment and/or prophylaxis of overactive bladder and symptoms related to overactive bladder, the compounds of the present invention can be administered in combination or as co-medication in addition to behavioural therapy like diet, lifestyle or bladder training with anticholinergics like oxybutynin, tolterodine, propiverine, solifenacin, darifenacin, trospium, fesoterdine; β-3 agonists like mirabegron; neurotoxins like onabutolinumtoxin A; or antidepressants like imipramine, duloxetine.

For the treatment and/or prophylaxis of interstitial cystitis, the compounds of the present invention can be administered in combination or as co-medication in addition to behavioural therapy like diet, lifestyle or bladder training with pentosans like elmiron; antidepressants like amitriptyline, imipramine; or antihistamines like loratadine.

For the treatment and/or prophylaxis of gynaecological diseases, the compounds of the present invention can be administered in combination or as co-medication with any substance that can be applied as therapeutic agent in the following indications:

dysmenorrhea, including primary and secondary; dyspareunia; endometriosis; endometriosis-associated pain; endometriosis-associated symptoms, such as and in particular dysmenorrhea, dyspareunia, dysuria, or dyschezia.

For the treatment and/or prophylaxis of dysmenorrhea, including primary and secondary; dyspareunia; endometriosis and endometriosis-associated pain, the compounds of the present invention can be administered in combination with ovulation inhibiting treatment, in particular COCs as mentioned above or contraceptive patches like Ortho-Evra or Apleek (Lisvy); or with progestogenes like dienogest (Visanne); or with GnRH analogous, in particular GnRH agonists and antagonists, for example leuprorelin, nafarelin, goserelin, cetrorelix, abarelix, ganirelix, degarelix; or with androgens: danazol.

For the treatment and/or prophylaxis of diseases, which are associated with pain, or pain syndromes, the compounds of the present invention can be administered in combination or as co-medication with any substance that can be applied as therapeutic agent in the following indications:

pain-associated diseases or disorders like hyperalgesia, allodynia, functional bowel disorders (such as irritable bowel syndrome) and arthritis (such as osteoarthritis, rheumatoid arthritis and ankylosing spondylitis), burning mouth syndrome, burns, migraine or cluster headache, nerve injury, traumatic nerve injury, post-traumatic injuries (including fractures and sport injuries), neuritis, neuralgia, poisoning, ischemic injury, interstitial cystitis, viral, trigeminal neuralgia, small fiber neuropathy, diabetic neuropathy, chronic arthritis and related neuralgias, HIV and HIV treatment-induced neuropathy.

The compounds of the present invention can be combined with other pharmacological agents and compounds that are intended to treat inflammatory diseases, inflammatory pain or general pain conditions.

In addition to well-known medicaments which are already approved and on the market, the compounds of the present invention can be administered in combination with inhibitors of the P2X purinoceptor family (P2X3, P2X4), with inhibitors of IRAK4 and with antagonists of the prostanoid EP4 receptor.

In particular, the compounds of the present invention can be administered in combination with pharmacological endometriosis agents, intended to treat inflammatory diseases, inflammatory pain or general pain conditions and/or interfering with endometriotic proliferation and endometriosis associated symptoms, namely with inhibitors of Aldo-keto-reductase1C3 (AKR1C3) and with functional blocking antibodies of the prolactin receptor.

The compounds of the present invention can be combined with other pharmacological agents and compounds that are intended for the treatment, prevention or management of cancer.

In particular, the compounds of the present invention can be administered in combination with 131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil +oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, or zorubicin.

Furthermore, the compounds of the present invention can be combined with active ingredients, which are well known for the treatment of cancer-related pain and chronic pain. Such combinations include, but are not limited to step II opiods like codeine phosphate, dextropropoxyphene, dihydro-codeine, Tramadol), step III opiods like morphine, fentanyl, buprenorphine, oxymorphone, oxycodone and hydromorphone; and other medications used for the treatment of cancer pain like steroids as Dexamethasone and methylprednisolone; bisphosphonates like Etidronate, Clodronate, Alendronate, Risedronate, and Zoledronate; tricyclic antidepressants like Amitriptyline, Clomipramine, Desipramine, Imipramine and Doxepin; class I antiarrhythmics like mexiletine and lidocaine; anticonvulsants like carbamazepine, Gabapentin, oxcarbazepine, phenytoin, pregabalin, topiramate, alprazolam, diazepam, flurazepam, pentobarbital and phenobarbital.

In addition to those mentioned above, the inventive Bradykinin B1 inhibitors can also be combined with any of the following active ingredients:

active ingredients for Alzheimer's therapy, for example acetylcholinesterase inhibitors (e.g. donepezil, rivastigmine, galantamine, tacrine), NMDA (N-methyl-D-aspartate) receptor antagonists (e.g. memantine); L-DOPA/carbidopa (L-3,4-dihydroxyphenylalanine), COMT (catechol-O-methyltransferase) inhibitors (e.g. entacapone), dopamine agonists (e.g. ropinrole, pramipexole, bromocriptine), MAO-B (monoaminooxidase-B) inhibitors (e.g. selegiline), anticholinergics (e.g. trihexyphenidyl) and NMDA antagonists (e.g. amantadine) for treatment of Parkinson's; beta-interferon (IFN-beta) (e.g. IFN beta-1b, IFN beta-1a Avonex® and Betaferon®), glatiramer acetate, immunoglobulins, natalizumab, fingolimod and immunosuppressants such as mitoxantrone, azathioprine and cyclophosphamide for treatment of multiple sclerosis; substances for treatment of pulmonary disorders, for example beta-2-sympathomimetics (e.g. salbutamol), anticholinergics (e.g. glycopyrronium), methylxanthines (e.g. theophylline), leukotriene receptor antagonists (e.g. montelukast), PDE-4 (phosphodiesterase type 4) inhibitors (e.g. roflumilast), methotrexate, IgE antibodies, azathioprine and cyclophosphamide, cortisol-containing preparations; substances for treatment of osteoarthritis such as non-steroidal anti-inflammatory substances (NSAIDs). In addition to the two therapies mentioned, methotrexate and biologics for B-cell and T-cell therapy (e.g. rituximab, abatacept) should be mentioned for rheumatoid disorders such as rheumatoid arthritis and juvenile idiopathic arthritis. Neurotrophic substances such as acetylcholinesterase inhibitors (e.g. donepezil), MAO (monoaminooxidase) inhibitors (e.g. selegiline), interferons and anticonvulsives (e.g. gabapentin); active ingredients for treatment of cardiovascular disorders such as beta-blockers (e.g. metoprolol), ACE inhibitors (e.g. benazepril), diuretics (e.g. hydrochlorothiazide), calcium channel blockers (e.g. nifedipine), statins (e.g. simvastatin); anti-diabetic drugs, for example metformin and glibenclamide, sulphonylureas (e.g. tolbutamide) and insulin therapy for treatment of diabetes and metabolic syndrome. Active ingredients such as mesalazine, sulfasalazine, azathioprine, 6-mercaptopurine or methotrexate, probiotic bacteria (Mutaflor, VSL#3®, Lactobacillus GG, Lactobacillus plantarum, L. acidophilus, L. casei, Bifidobacterium infantis 35624, Enterococcus fecium SF68, Bifidobacterium longum, Escherichia coli Nissle 1917), antibiotics, for example ciprofloxacin and metronidazole, anti-diarrhoea drugs, for example loperamide, or laxatives (bisacodyl) for treatment of chronic-inflammatory bowel disorders. Immunosuppressants such as glucocorticoids and non-steroidale anti-inflammatory substances (NSAIDs), cortisone, chloroquine, cyclosporine, azathioprine, belimumab, rituximab, cyclophosphamide for treatment of lupus erythematosus. By way of example but not exclusively, calcineurin inhibitors (e.g. tacrolimus and ciclosporin), cell division inhibitors (e.g. azathioprine, mycophenolate mofetil, mycophenolic acid, everolimus or sirolimus), rapamycin, basiliximab, daclizumab, anti-CD3 antibodies, anti-T-lymphocyte globulin/anti-lymphocyte globulin for organ transplants, Vitamin D3 analogues, for example calcipotriol, tacalcitol or calcitriol, salicylic acid, urea, ciclosporine, methotrexate, or efalizumab for dermatological disorders.

### Methods of Treating

The present invention relates to a method for using the compounds of the present invention and compositions thereof, to inhibit the Bradykinin B1 receptor.

The present invention relates to a method for using the compounds of the present invention and compositions thereof, to treat mammalian disorders and diseases which include but are not limited to:

Diseases related to pain and inflammation, in particular selected from the group consisting of
visceral pain e.g. related to pancreatitis, interstitial cystitis, renal colic, or prostatitis, chronic pelvic pain, or pain related to infiltrating endometriosis;
neuropathic pain such as post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
central pain syndromes potentially caused by virtually any lesion at any level of the nervous system including but not limited to pain related to stroke, multiple sclerosis, and spinal cord injury;
postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative, and perioperative pain (including but not limited to general surgery, orthopaedic, and gynaecological surgery); and
acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis);
and diseases like or related to a disease selected from the group consisting of:
gynaecological disorders and/or diseases, or effects and/or symptoms which negatively influence women health including endometriosis, uterine fibroids, pre-eclampsia, hormonal deficiency, spasms of the uterus, or heavy menstrual bleeding;
the respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
dermatology including pruritus, itch, inflammatory skin disorders including psoriasis, eczema, and atopic dermatitis;
affection of the joints or bones including rheumatoid arthritis, gout, osteoporosis, osteoarthritis, and ankylosing spondylitis;
affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
infection including HIV infection, and tuberculosis;
trauma associated with oedema including cerebral oedema, burns, sunburns, and sprains or fracture;
poisoning including aluminosis, anthracosis, asbestosis, chalicosis, ptilosis, siderosis, silicosis, tabacosis, and byssinosis uveitis;
diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, or fat or muscle metabolism;
cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
cardio-vascular system including congestive heart failure, atherosclerosis, congestive heart failure, myocardial infarct, and heart fibrosis; and
other conditions including septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.

A preferred embodiment of the present invention relates to a method for using the compounds of the present invention and compositions thereof, to treat a gynaecological disease, preferably dysmenorrhea, dyspareunia or endometriosis, endometriosis-associated pain, or other endometriosis-associated symptoms, wherein said symptoms include dysmenorrhea, dyspareunia, dysuria, or dyschezia. Additionally the present invention relates to a method for using the compounds of the present invention and compositions thereof, to treat osteoarthritis, rheumatoid arthritis, gout, neuropathic pain, asthma, cough, lung injury, lung fibrosis, pneumonia, kidney fibrosis, kidney failure pruritus, irritable bowel disease, overactive urinary bladder, diabetes type 1, diabetes type 2, diabetic neuropathy, diabetic retinopathy, diabetic macular oedema, metabolic syndrome, obesity, heart fibrosis, cachexia, muscle atrophy, Alzheimer's disease, and interstitial cystitis.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a gynaecological disease.

### Dose and Administration

Based upon standard laboratory techniques known to evaluate compounds useful for the treatment of disorders and/or diseases which are mediated by Bradykinin B1 receptor, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed the mode of administration, the period of treatment, the age and sex of the patient treated, and the nature and extent of the condition treated.

The total amount of the active ingredient to be administered will generally range from about 0.001 mg/kg to about 200 mg/kg body weight per day, preferably from about 0.01 mg/kg to about 20 mg/kg body weight per day. A preferred administration of the compound of the present invention includes but is not limited to 0.1 mg/kg to about 10 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. A preferred oral unit dosage for administration of the compounds of the present invention includes but is not limited to 0.1 mg/kg to about 10 mg/kg body weight one to three times a day to once a week. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg of total body weight. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases treated with said method are gynaecological disorders, more preferably dysmenorrhea, dyspareunia or endometriosis, endometriosis-associated pain, or other endometriosis-associated symptoms, wherein said symptoms include dysmenorrhea, dyspareunia, dysuria, or dyschezia. Further diseases which can be treated with said method are osteoarthritis, rheumatoid arthritis, gout, neuropathic pain, asthma, cough, lung injury, lung fibrosis, pneumonia, kidney fibrosis, kidney failure pruritus, irritable bowel disease, overactive urinary bladder, diabetes type 1, diabetes type 2, diabetic neuropathy, diabetic retinopathy, diabetic macular oedema, metabolic syndrome, obesity, heart fibrosis, cachexia, muscle atrophy, Alzheimer's disease, and interstitial cystitis.

Preferably, the method of treating the diseases mentioned above is not limited to the treatment of said disease but also includes the treatment of pain related to or associated with said diseases.

The compounds of the present invention can be used in particular in therapy and prevention, i.e. prophylaxis, of genitourinary, gastrointestinal, respiratory or pain-related disease, condition or disorder.

Methods of testing for a particular pharmacological or pharmaceutical property are well known to persons skilled in the art.

The example testing experiments described herein serve to illustrate the present invention and the invention is not limited to the examples given.

### Synthesis of Compounds of General Formula (I) of the Present Invention

Compounds of general formula (I) with the meaning of R¹, R⁵ and A, X, R^{d}, R^{e}, R³ and R⁴ as defined in general formula (I), can be synthesised according to various general procedures, some of which are exemplified below.

Scheme 1 depicts the synthesis starting from synthons of the formula (II), wherein Hal stands for Cl, Br or I, Br being preferred. The aryl halides of the general formula (II) can be cross-coupled with boronic acids of the general formula (III) or alternatively with their respective pinacol esters to yield compounds of general formula (IV) by Pd-mediated reactions (Suzuki coupling) known to those skilled in the art. A suitable solvent (for example N,N-dimethylformamide, tetrahydrofuran, 1,4-dioxane, dimethoxyethane and optionally water) is used and a base (such as triethylamine, potassium carbonate, caesium carbonate) and a catalyst-ligand mixture, for example of palladium(II) acetate/triphenylphosphine, tetrakis(triphenylphosphine)palladium(0), bis(triphenylphosphine) palladium(II) dichloride, bis(diphenylphosphino)ferrocenedichloro-palladium (II) is utilised at temperatures between 20° C. and 120° C., preferred at 100° C.

The nitrile moiety of formula (IV) is converted to tetrazoles of the general formula (V) by reaction with 1-4 equivalents of trimethylsilyl azide in the presence of 1-2 equivalents of dibutyltinoxide in toluene or xylene as solvent at temperatures between 50° C. and 160° C. Any tetrazole moieties shown in chemical formulas herein are for illustrative purposes and have to be understood as both 1H- and 2H-tautomers.

Aromatic amines of formula (V) may react with carboxylic acid of formula (VI) by methods known to those skilled in the art to give the compounds of the general formula (I). The reaction is mediated by activating a carboxylic acid of formula (VI) with suited reagents such as dicyclohexylcarbodiimide (DCC), 1-(3-dimethylaminopropyl)-3-ethylcarbodiimide (EDCI), N-hydroxybenzotriazole (HOBT), N-[(dimethylamino)-(3H-[1,2,3]triazolo[4,5-b]pyridin-3-yloxy)methyliden]-N-methylmethanaminium hexafluorophosphate (HATU) or propylphosphonic anhydride (T3P). For example, the reaction with HATU takes place in an inert solvent, such as N,N-dimethylformamide, dichloromethane or dimethyl sulfoxide in the presence of the appropriate amine formula (V) and a tertiary amine (such as triethylamine or diisopropylethylamine) at temperatures between −30° C. and +60° C., preferably at room temperature.

It is also possible to convert a carboxylic acid of the formula (VI) into the corresponding carboxylic acid chloride with an inorganic acid chloride (such as phosphorus pentachloride, phosphorus trichloride or thionyl chloride) and then into the target compounds of the general formula (I), in pyridine or an inert solvent (such as N,N-dimethylformamide), in the presence of the appropriate amine formula (V) and a tertiary amine (for example triethylamine) at temperatures between −30° C. and +60° C., preferably between 0° C. and room temperature.

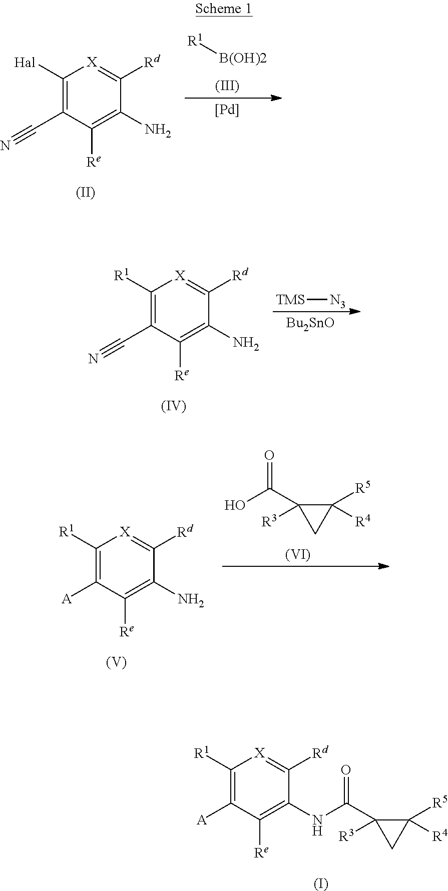

Scheme 2 shows an alternative approach in which the sequence of reaction steps is changed. Also starting from synthons of the general formula (II), first the tetrazole (A) is formed yielding compounds of the general formula (VII). To the NH of the tetrazole (A), a protecting group is attached. A suitable protecting group is e.g. the 2-(trimethylsilyl)ethoxymethyl group (SEM). To attach the SEM-group, the tetrazole (A) compound (VII) is reacted with 1-1.5 equivalents 2-(trimethylsilyl)ethoxymethyl chloride in the presence of a base, e.g. N,N-diisopropylethylamine (1-2 equivalents) in a solvent like e.g. N,N-dimethylformide. A separable mixture of both possible SEM-regioisomers is obtained.

In analogy to the procedures described for Scheme 1, amide coupling gives compounds of the general formula (IX), followed by the Suzuki reaction which yields SEM-protected compounds of the general formula (X). Cleavage of the SEM group can be accomplished either by reaction with 1.2 equivalents of tetra-n-butylammonium fluoride in dichloromethane or alternatively by heating to 60° C. in 3 M hydrochloric acid/methanol 1:1 for ca. 1 h, to yield the target compounds of general formula (I).

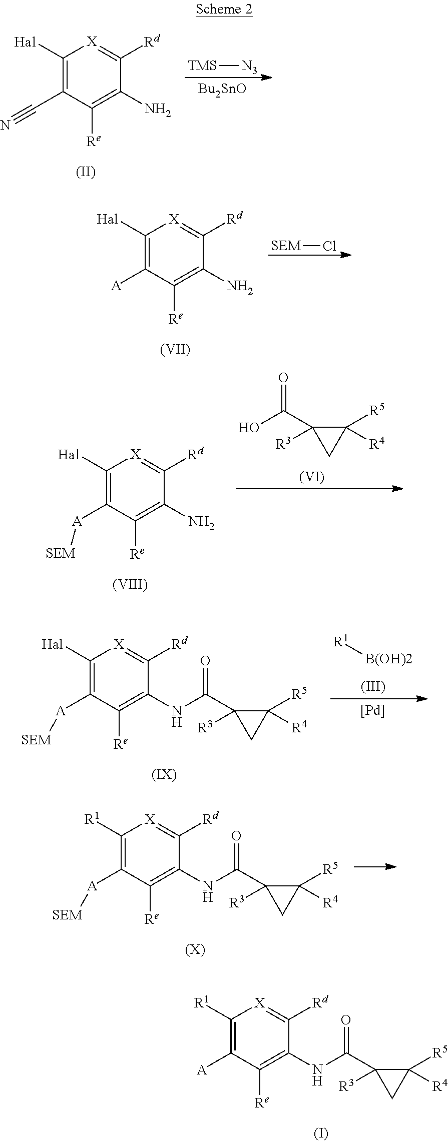

The starting materials of the general formula (II) are either commercially available or can be synthesized via methods known to those skilled in the art from appropriate precursors. For example, the amino group may be obtained by reduction of the corresponding nitro group with hydrogen in the presence of a palladium catalyst in solvents like ethanol, ethyl acetate or mixtures thereof. Alternatively, the amino group may be obtained by reduction of the corresponding nitro group with Iron/HCl. The nitro group may be introduced by classical methods like treatment with nitric acid/sulphuric acid (with appropriate concentration and volume ratio) at temperatures between 0° C. and 25° C. The sequence of reactions steps (nitro reduction, Suzuki reaction, tetrazole formation) may be changed as appropriate.

Scheme 3 shows an alternative approach to synthesis of compounds of general formula (I) where R¹ is an N-linked optionally substituted 5-membered heteroaryl or 4- to 7-membered heterocycloalkyl group, for example pyrazolyl, imidazolyl or pyrrolidinyl. Starting from synthons of the general formula (XI) (wherein Hal stands for Cl, Br or I) the aryl halide can first be substituted by a nucleophile of general formula (XII) to yield a compound of general formula (XIII). R¹ in formula (XII) is linked to the H through a nitrogen atom. The substitution takes place in a dipolar aprotic solvent such as acetonitrile, DMSO or DMF and in the presence of an appropriate base (for example potassium carbonate) at temperatures between RT and 100° C., preferably at 60° C. The nitrile group of formula (XIII) can subsequently be converted to a tetrazole of the general formula (XIV) by reaction with 1-4 equivalents of trimethylsilyl azide in the presence of 1-2 equivalents of dibutyltinoxide in toluene or xylene solvent at temperatures between 50° C. and 160° C., preferably between 120° C. and 140° C. The tetrazoles of the present invention have to be understood as both 1H- and 2H-tautomers. The nitro group of a compound of general formula (XIV) is then reduced to the corresponding aniline of general formula (V) by reaction under a hydrogen atmosphere in the presence of a palladium catalyst (for example 5-10% palladium on carbon) in an appropriate solvent (for example ethanol or ethyl acetate) at temperatures between 0° C. and 100° C. In analogy to the procedures described for Scheme 1, amide coupling gives compounds of the general formula (I).

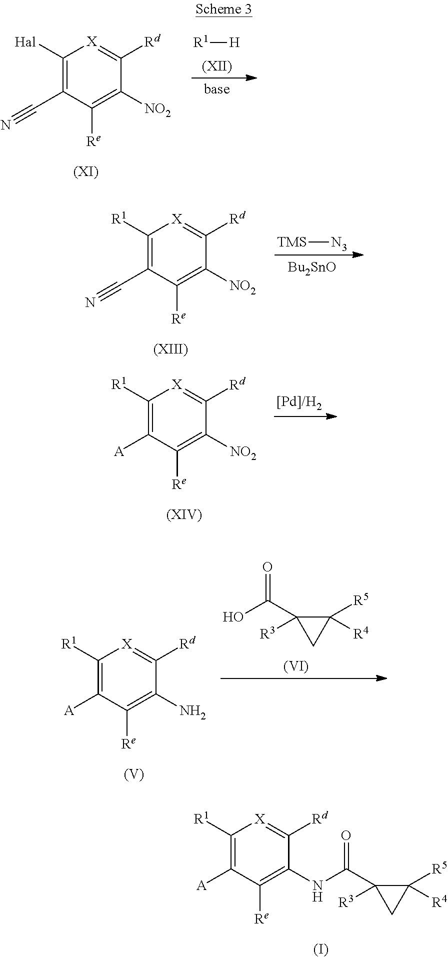

Alternatively to Scheme 3, the reaction sequence can be modified as depicted in Scheme 4 to synthesise compounds of general formula (I) where R¹ is an N-linked optionally substituted 4- to 7-membered heterocycloalkyl or 5-membered heteroaryl, for example pyrazolyl, imidazolyl or pyrrolidinyl. R¹ in formula (XII) is linked to the H through a nitrogen atom.

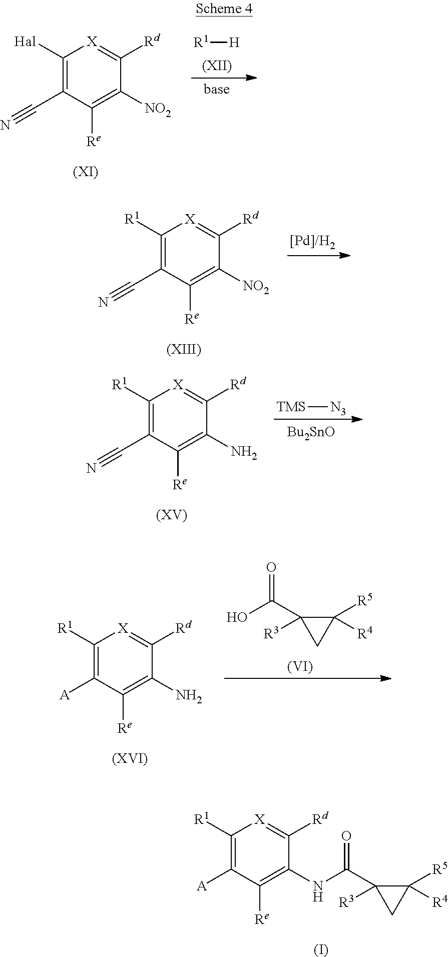

The carboxylic acids of the general formula (VI) are either commercially available or can be synthesized from appropriate precursors via methods known to those skilled in the art. For example, Scheme 5 depicts the synthesis starting from oxirane of general formula (XVII). The oxirane synthon is reacted with the appropriate compound of general formula (XVIII) (for example triethyl 2-phosphonopropionate) in the presence of n-butyllithium in a suitable aprotic solvent (for example 1,2-dimethoxyethane) at temperatures between −20° C. and +5° C. (typically at 0° C.), followed by heating at temperatures between 60° C. and 120° C. (typically at 100° C.). The ester group of formula (XIX) can subsequently be converted to a carboxylic acid of the general formula (VI) in aqueous base (such as lithium hydroxide) in an appropriate solvent (for example THF) at temperatures between 0° C. and 60° C., usually and in particular at room temperature.

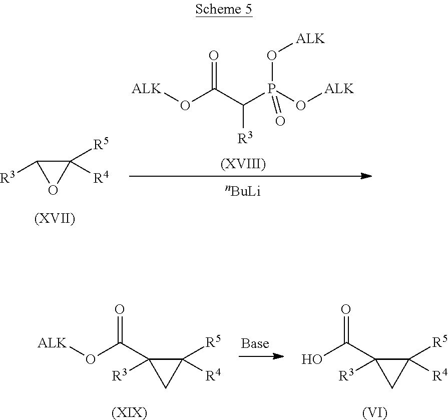

Scheme 6 shows an alternative approach to carboxylic acid intermediates of general formula (VI). An alkene of the general formula (XX) can be reacted with an α-alkyl-α-diazoester of general formula (XXI) in the presence of a metal catalyst (for example dirhodium tetraacetate) in a suitable solvent (for example dichloromethane, 1,2-dichloroethane or tetrahydrofuran) at a temperature between 0° C. and 40° C. The ester group of formula (XXII) can subsequently be converted to a carboxylic acid of the general formula (VI) in aqueous base (such as lithium hydroxide) in an appropriate solvent (for example THF) at temperatures between 0° C. and 60° C., usually at room temperature.

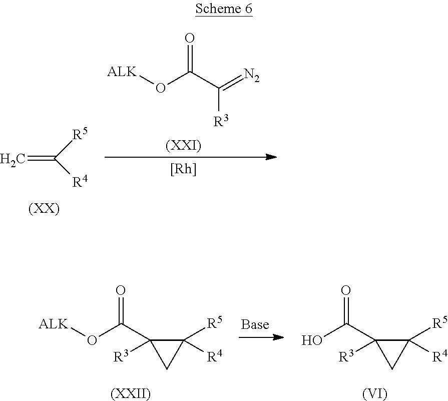

Scheme 7 shows an alternative approach to synthesis of compounds of general formula (I) where R¹ is a substituted cyclohexyl group. Starting from synthons of the general formula (XI) (wherein Hal stands for Cl, Br or I; Br being preferred) the aryl halide can first be reacted with a cross-coupling partner of general formula (XIII) (wherein X³ is SnBu_{3,} B(OH)₂ or the respective pinacol boronic ester) to yield a compound of general formula (XXIV). A suitable solvent (for example N,N-dimethylformamide, tetrahydrofuran, 1,4-dioxane, dimethoxyethane, toluene and optionally water) is used and a catalyst-ligand mixture, for example of palladium(II) acetate/triphenylphosphine, tetrakis(triphenylphosphine)-palladium(0), tris(dibenzylideneacetone)dipalladium, or bis(triphenylphosphine)-palladium(II) dichloride, bis(diphenylphosphino)ferrocenedichloropalladium (II) is utilised at temperatures between 10° C. and 120° C. The resulting styrene of general formula (XXIV) can then be converted into a corresponding cyclohexanone of general formula (XXVI) by Diels-Alder reaction with a suitable diene of general formula (XXV) (for example 2-trimethylsiloxy-1,3-butadiene) in a suitable solvent (for example toluene or xylene) at temperatures between 10° C. and 180° C. Subsequent hydrolysis of the resulting silyl enol ether is achieved with aqueous acid (i.e 1 to 6 molar aqueous hydrochloric acid) where applicable. Reduction of a ketone of general formula (XXVI) is achieved using a reducing agent (for example sodium borohydride) in a suitable solvent (such as methanol or tetrahydrofuran) at temperatures between −40° C. to 100° C. The resulting alcohol of general formula (XXVII) can then be alkylated with an alkyl halide of general formula (XXVIII) (wherein Hal stands for Cl, Br or I and R² is C₁-C₅-alkyl, optionally substituted with OH, OR² or 1 to 5 fluorine atoms) in the presence of a suitable base (for example sodium hydride or potassium tert-butoxide) in an appropriate solvent (such as dimethylformamide or dioxane) at temperature between −40° C. and 100° C. The nitrile moiety of formula (XXIX) can subsequently be converted to a tetrazole of the general formula (XXX) by reaction with 1 to 4 equivalents of trimethylsilyl azide in the presence of 1-2 equivalents of dibutyltinoxide in toluene or xylene solvent at temperatures between 50° C. and 160° C. (typically between 120° C. and 140° C.). The tetrazoles of the present invention have to be understood as both 1H- and 2H-tautomers. The nitro group of a compound of general formula (XXX) is then reduced to the corresponding aniline of general formula (XXXI) by reaction under a hydrogen atmosphere in the presence of a palladium catalyst (for example 5-10% palladium on carbon) in an appropriate solvent (for example ethanol or ethyl acetate) at temperatures between 0° C. and 100° C. (typically at room temperature). In analogy to the procedures described for Scheme 1, amide coupling gives compounds of the general formula (I).

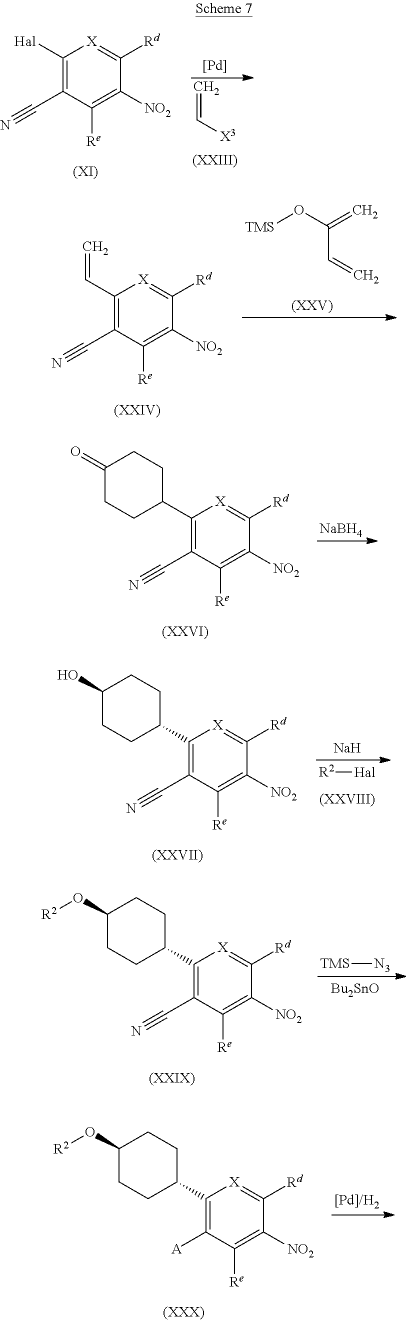

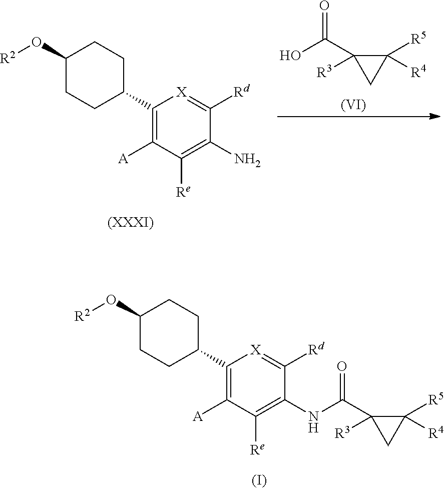

### Experimental Section

The example testing experiments described herein serve to illustrate the present invention and the invention is not limited to the examples given.

The following table lists the abbreviations used in this paragraph, in the examples section and biological assays section.

| Abbreviation | Meaning |
|---|---|
| ALK | Alkyl group |
| Bu₂SnO | Dibutyltinoxide |
| BSA | Bovine Serum Albumin |
| Cs₂CO₃ | Cesium carbonate |
| ca. | circa |
| DCE | 1,2-Dichloroethane |
| DCM | Dichloromethane |
| DIPEA | N-Ethyl-N-isopropylpropan-2-amine |
| DMAP | N,N-Dimethylpyridin-4-amine |
| DME | 1,2-Dimethoxyethane |
| DMF | N,N-Dimethylformamide |
| DMSO | Dimethyl sulfoxide |
| DP | Desired product |
| EtOAc | Ethyl acetate |
| EE | Ethyl acetate |
| h | Hour |
| HATU | N-[(Dimethylamino)(3H-[1,2,3]triazolo[4,5- |
| | b]pyridin-3-yloxy)methylene]-N- |
| | methylmethanaminium hexafluorophosphate |
| HCl | Hydrochloric acid |
| HPLC | high performance liquid chromatography |
| IPC | In process check |
| K₂CO₃ | Potassium carbonate |
| LC-MS | liquid chromatography - mass spectrometry |
| LCMS | liquid chromatography - mass spectrometry |
| LiOH | Lithium hydroxide |
| M | Molar |
| μW | Microwave |
| MeCN | Acetonitile |
| MeOH | Methanol |
| MgSO₄ | Magnesium sulfate |
| min | Minute(s) |
| N | Normal |
| Na₂CO₃ | Sodium carbonate |
| NaH | Sodium hydride |
| NaHCO₃ | Sodium bicarbonate |
| NaI | Sodium iodide |
| NaOH | Sodium hydroxide |
| Na₂SO₄ | Sodium sulfate |
| NMR | nuclear magnetic resonance spectroscopy |
| PdCl₂(PPh₃)₂ | Bis(triphenylphosphine)palladium(II) dichloride |
| Pd(dppf)Cl₂ | [1,1′-Bis(diphenylphosphino)ferrocene]- |
| | dichloropalladium(II) |
| Pd(dppf)Cl₂•CH₂Cl₂ | 1,1′-Bis(diphenylphosphino)ferrocene- |
| | palladium(II)dichloride dichloromethane complex |
| PPh₃ | Triphenylphosphine |
| ppm | parts per million |
| Py | Pyridine |
| RFU | Relative fluorescence units |
| RT | Room temperature |
| rt | Retention time |
| Rt | Retention time |
| [Rh] | Dirhodium tetraacetate |
| sat. | Saturated |
| SEM | 2-(Trimethylsilyl)ethoxymethyl |
| SFC | Supercritical fluid chromatography |
| SiO₂ | Silica gel |
| SM | Starting material |
| TMS-azide | Azidotrimethylsilane |
| TMS-N₃ | Azidotrimethylsilane |
| T3P | Propylphosphonic anhydride |
| TEA | Triethylamine |
| TFA | Trifluoroacetic acid |
| THF | Tetrahydrofuran |

### Analysis Methods

### Analytical LCMS Methods

Method 1: Instrument: Waters Acquity Platform ZQ4000; column: Waters BEHC 18, 50 mm×2.1 mm, 1.7 μm; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.
Method 2: Instrument: Waters Acquity LCT; column: Phenomenex Kinetex C18, 50 mm×2.1 mm, 2.6 μm; eluent A: water/0.05% formic acid, eluent B: acetonitrile/0.05% formic acid; gradient: 0.0 min 98% A 0.2 min: 98% A→4 1.7 min: 10% A→1.9 min: 10% A→2 min: 98% A→2.5 min: 98% A; flow: 1.3 ml/min; column temperature: 60° C.; UV-detection: 200-400 nm.
Method 3: Instrument: Waters Acquity UPLCMS SingleQuad; Column: Acquity UPLC BEH C18 1.7 μm, 50×2.1mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.
Method 4: Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.
Method 5: Instrument MS: Waters ZQ; instrument HPLC: Waters UPLC Acquity; column: Acquity BEH C18 (Waters), 50 mm×2.1 mm, 1.7 μm; eluent A: water +0.1% formic acid, eluent B: acetonitrile (Lichrosolv Merck); gradient: 0.0 min 99% A-1.6 min 1% A-1.8 min 1%A-1.81 min 99% A-2.0 min 99% A; oven: 60° C.; flow: 0.800 ml/min; UV-detection PDA 210-400 nm.

### LC-MS, Analytical Method A: Routine High Throughput Analysis

Column: Kinetex Core-Shell C18, 2.1×50 mm, 5 μm; Eluent A: Water+0.1% Formic acid, Eluent B: Acetonitrile+0.1% Formic acid; Gradient 0.00 mins 95% A→1.20 mins 100% B→1.30 mins 100% B-1.31 mins 95% A; column temperature: 40° C.; flow rate 1.2 ml/min; injection volume: 3 μl; UV-detection range: 210-420 nm.

### LC-MS, Analytical Method C: Routine High Throughput Analysis at High pH

Column: Phenomenex Gemini-NX C18, 2.0×50 mm, 3 μm; Eluent A: 2 mM ammonium bicarbonate, buffered to pH10, Eluent B: Acetonitrile; Gradient 0.00 mins 99% A→1.80 mins 100% B→2.10 mins 100% B→2.30 mins 99% A→3.50 mins 99% A; column temperature: 40° C.; flow rate 1.0 ml/min; injection volume: 3 μl; UV-detection range: 210-420 nm.

### LC-MS, Analytical Method D:

Column: Waters Atlantis dC18, 2.1×100 mm, 3 μm; Eluent A: Water+0.1% Formic acid, Eluent B: Acetonitrile+0.1% Formic acid; Gradient 0.00 mins 95% A→5.00 mins 100% B→5.40 mins 100% B→5.42 mins 95% A→7.00 mins 95% A; column temperature: 40° C.; flow rate 0.6 ml/min; injection volume: 3 μl; UV-detection range: 210-420 nm.

### LC-MS, Analytical Method E: High pH

Column: Phenomenex Gemini-NX C18, 2.0×100 mm, 3 μm; Eluent A: 2 mM ammonium bicarbonate, buffered to pH10, Eluent B: Acetonitrile; Gradient 0.00 mins 95% A→5.50 mins 100% B→5.90 mins 100% B→5.92 mins 95% A→7.00 mins 95% A; column temperature: 40° C.; flow rate 0.5 ml/min; injection volume: 3 μl; UV-detection range: 210-420 nm.

### LC-MS, Analytical Method F:

Column: Phenomenex Kinetix-XB C18, 2.1×100 mm, 1.7 μm; Eluent A: Water+0.1% Formic acid, Eluent B: Acetonitrile+0.1% Formic acid; Gradient 0.00 mins 95% A→5.30 mins 100% B→5.80 mins 100% B→5.82 mins 95% A→7.00 mins 95% A; column temperature: 40° C.; flow rate 0.6 ml/min; injection volume: 1 μl; UV-detection range: 200-400 nm.

### Purification Methods:

Biotage Isolera™ chromatography system using pre-packed silica and pre-packed modified silica cartridges.

### Preparative HPLC, Method A: High pH

Column: Waters Xbridge C18, 30×100 mm, 10 μm; Solvent A: Water+0.2% Ammonium hydroxide, Solvent B: Acetonitrile+0.2% Ammonium hydroxide; Gradient 0.00 mins 90% A→0.55 mins 90% A→14.44 mins 95% B→16.55 mins 95% B→16.75 90% A; column temperature: room temperature; flow rate 40 ml/min; injection volume: 1500 μl; Detection: UV 215 nm.

### Preparative HPLC, Method B: Low pH

Column: Waters Sunfire C18, 30×100 mm, 10 μm; Solvent A: Water +0.1% Formic acid, Solvent B: Acetonitrile+0.1% Formic acid; Gradient 0.00 mins 90% A→4 0.55 mins 90% A→14.44 mins 95% B→16.55 mins 95% B→16.75 90% A; column temperature: room temperature; flow rate 40 ml/min; injection volume: 1500 82 l; Detection: UV 215 nm.

### Preparative HPLC Methods

Preparative HPLC, method 1:
System: Waters autopurification system: Pump 2545, Sample Manager 2767, CFO, DAD 2996, ELSD 2424, SQD; Column: XBrigde C18 5 μm 100×30 mm; Solvent: A=H2O+0.1% Vol. formic acid (99%), B=acetonitrile; Gradient: 0-8 min 10-100% B, 8-10 min 100% B; Flow: 50 mL/min; temperature: room temp.; Solution: Max. 250 mg/max. 2.5 mL DMSO or DMF; Injection: 1×2.5 mL; Detection: DAD scan range 210-400 nm; MS ESI+, ESI−, scan range 160-1000 m/z.

### Preparative HPLC, Method 2:

System: Waters autopurification system: Pump 2545, Sample Manager 2767, CFO, DAD 2996, ELSD 2424, SQD; Column: XBrigde C18 5 μm 100×30 mm; Solvent: A=H2O+0.1% Vol. ammonia (99%), B=acetonitrile; Gradient: 0-8 min 10-100% B, 8-10 min 100% B; Flow: 50 mL/min; temperature: room temp.; Solution: Max. 250 mg/max. 2.5 mL DMSO o. DMF; Injection: 1×2.5 mL; Detection: DAD scan range 210-400 nm; MS ESI+, ESI−, scan range 160-1000 m/z.

### EXAMPLES

Chemical naming of the Examples and Intermediates was performed using either ACD-naming software (by ACD/LABS) or Marvin software (by ChemAxon).

Reaction times are either specified explicitly in the protocols of the experimental section, or reactions were run until completion. Chemical reactions were monitored and their completion was judged using methods well known to the person skilled in the art, such as thin layer chromatography, e.g. on plates coated with silica gel, or by LCMS methods.

### Intermediate 1A: 4-amino-3′,4′-dimethoxybiphenyl-2-carbonitrile

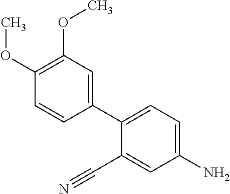 [SMILES: COc1ccc(-c2ccc(N)cc2C#N)cc1OC]

A solution of 5-amino-2-bromobenzonitrile (5.0 g, 25.3 mmol), (3,4-dimethoxyphenyl)boronic acid (5.1 g, 27.8 mmol) and potassium carbonate (11.6 g, 84 mmol) in dimethoxyethane (75 mL) and water (30 mL) was degassed with a stream of nitrogen gas for 5 mins. Dichlorobis(triphenylphosphine)palladium(II) (178 mg, 0.25 mmol) was added, and the reaction heated at 100° C. for 60 min. The reaction was then cooled to RT and diluted with EE (50 mL) and the aqueous layer was removed. The organics were washed with brine (2×40 mL), dried (Na₂SO₄), filtered and concentrated to give the desired product (6.4 g, quant. yield) as an orange solid, which was used without further purification.

¹H NMR (500 MHz, Chloroform-d) δ [ppm]7.29 (d, J=8.4 Hz, 1H), 7.08-7.04 (m, 2H), 7.00 (d, J=2.5 Hz, 1H), 6.94 (d, J=8.0 Hz, 1H), 6.91 (dd, J=8.4, 2.5 Hz, 1H), 3.94 (s, 3H), 3.92 (s, 3H), 3.87 (s, 2H);
LCMS (Analytical Method A) 100%@ Rt=1.13, MS (ESIpos) m/z=255 (M+H)⁺.

In analogy to the procedure described for Intermediate 1A, the following intermediates were prepared using 5-amino-2-bromobenzonitrile and the appropriate boronic acids or, respectively, the corresponding pinacol boronic esters as starting materials.

| Int. | Structure | Name | Analytical Data |
|---|---|---|---|
| 2A | 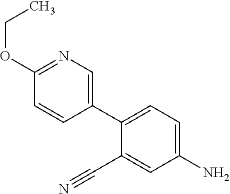 [SMILES: CCOc1ccc(-c2ccc(N)cc2C#N)cn1] | 5-amino-2-(6- ethoxypyridin- 3-yl)benzonitrile | ¹H NMR (400 MHz, DMSO-d6) δ [ppm] 1.34 (t, 3H), 4.34 (q, 2H), 5.75 (s, 2H), 6.88 (d, 1H), 6.93 (dd, 1H), 6.98 (d, 1H), 7.27 (d, 1H), 7.81 (dd, 1H), 8.24 (d, 1H). LCMS (method 1): Rₜ = 0.95, MS (ESIpos) m/z = 240 (M + H)⁺. |
| 3A | 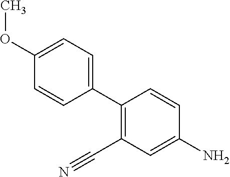 [SMILES: COc1ccc(-c2ccc(N)cc2C#N)cc1] | 4-amino-4′- methoxybipheny 1-2-carbonitrile | ¹H NMR (500 MHz, DMSO-d6) δ [ppm] 7.44-7.35 (m, 2H), 7.22 (d, J = 8.4 Hz, 1H), 7.06-6.98 (m, 2H), 6.96- 6.88 (m, 2H), 5.62 (s, 2H), 3.79 (s, 3H). LCMS (Analytical Method A): Rt = 1.07 min, MS (ESlpos) m/z = 225 (M + H)⁺. |
| 4A | 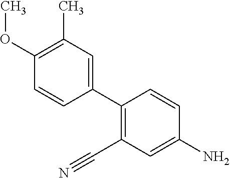 [SMILES: COc1ccc(-c2ccc(N)cc2C#N)cc1C] | 4-amino-4′- methoxy-3′- methylbiphenyl- 2-carbonitrile | ¹H NMR (500 MHz, DMSO-d6) δ [ppm] 2.19 (s, 3H), 3.82 (s, 3H), 5.60 (s, 2H), 6.89 (dd, J = 8.4, 2.4 Hz, 1H), 6.93 (d, J = 2.4 Hz, 1H), 7.00 (d, J = 8.4 Hz, 1H), 7.19-7.28 (m, 3H). LCMS (Analytical Method A): Rt = 1.14 min, MS (ESIpos) m/z = 239 (M + H)⁺. |
| 5A | 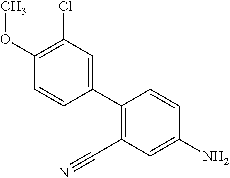 [SMILES: COc1ccc(-c2ccc(N)cc2C#N)cc1Cl] | 4-amino-3′- chloro-4′- methoxybipheny 1-2-carbonitrile | ¹H NMR (250 MHz, CDCl₃) δ [ppm] 7.48 (d, J = 2.3 Hz, IH), 7.42 (dd, J = 8.5. 2.3 Hz, 1H), 7.24 (d, J = 8.4 Hz, 1H), 7.04-6.97 (m, 2H), 6.90 (dd, J = 8.4, 2.6 Hz, 1H), 3.95 (s, 3H), 3.90 (s, 2H). LCMS (Analytical Method A): Rt = 1.14 min. MS (ESIpos) m/z = 259 (M + H)⁺. |
| 6A | 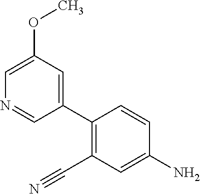 [SMILES: COc1cncc(-c2ccc(N)cc2C#N)c1] | 5-amino-2-(5- methoxypyridin- 3-yl)benzonitrile | ¹H NMR (500 MHz, DMSO-d6) δ [ppm] 8.28 (m, 2H), 7.51-7.44 (m, 1H), 7.36 (d, J = 8.5 Hz, 1H), 7.00 (d, J = 2.4 Hz, 1H), 6.95 (dd, J = 8.5, 2.4 Hz, 1H), 5.82 (s, 2H), 3.88 (s, 3H). LCMS (Analytical Method A): Rt = 0.81 min. MS (ESIpos) m/z = 226 (M + H)⁺. |
| 7A | 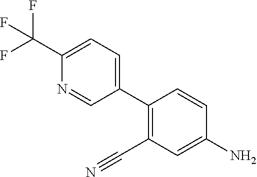 [SMILES: N#Cc1cc(N)ccc1-c1ccc(C(F)(F)F)nc1] | 5-amino-2-[6- (trifluoromethyl) pyridin-3-yl] benzonitrile | ¹H NMR (500 MHz, DMSO-d6) δ [ppm] 5.96 (s, 2H), 6.98 (dd, J = 8.5, 2.4 Hz, 1H), 7.05 (d, J = 2.4 Hz, 1H), 7.43 (d, J = 8.5 Hz, 1H), 8.00 (d, J = 8.2 Hz, 1H), 8.21 (dd, J = 8,2, 2.2 Hz, 1H), 8.88 (d, J = 2.1 Hz, 1H). LCMS (Analytical Method A): Rt = 1.10 min. MS (ESIpos) m/z = 264 (M + H)⁺. |
| 8A | 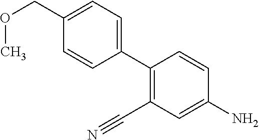 [SMILES: COCc1ccc(-c2ccc(N)cc2C#N)cc1] | 4-amino-4′- (methoxymethyl) biphenyl-2- carbonitrile | ¹H NMR (500 MHz, Methanol-d4) δ [ppm] 7.49 (d, J = 8.2 Hz, 2H), 7.43 (d, J = 8.2 Hz, 2H), 7.29 (d, J = 8.4 Hz, 1H), 7.05 (d, J = 2.4 Hz, 1H), 7.00 (dd, J = 8.4, 2.5 Hz, 1H), 4.53 (s, 2H), 3.42 (s, 3H). LCMS (Analytical Method A): Rt = 1.10 min. MS (ESIpos) m/z = 239 (M + H)⁺. |
| 9A | 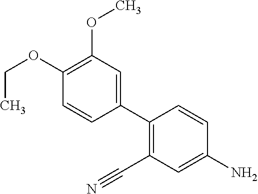 [SMILES: CCOc1ccc(-c2ccc(N)cc2C#N)cc1OC] | 4-amino-4′- ethoxy-3′- methoxybipheny 1-2-carbonitrile | ¹H NMR (500 MHz, DMSO-d6) δ [ppm] 1.34 (t, J = 7.0 Hz, 3H), 3.80 (s, 3H), 4.04 (q, J = 7.0 Hz, 2H), 5.63 (s, 2H), 6.88-7.03 (m, 4H), 7.05 (d, J = 2.0 Hz, 1H), 7.27 (d, J = 8,4 Hz, 1H). LCMS (Analytical Method A): Rt = 1.08 min. MS (ESlpos) m/z = 269 (M + H)⁺. |
| 10A | 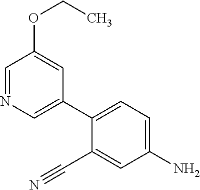 [SMILES: CCOc1cncc(-c2ccc(N)cc2C#N)c1] | 5-amino-2-(5- ethoxypyridin- 3-yl)benzonitrile | ¹H NMR (500 MHz, DMSO-d6) δ [ppm] 8.27 (d, J = 2.7 Hz, 1H), 8.25 (d, J = 1.8 Hz, 1H), 7.48-7.42 (m, 1H), 7.35 (d, J = 8.5 Hz, 1H), 7.00 (d, J = 2.4 Hz, 1H), 6.94 (dd, J = 8.5, 2.4 Hz, 1H), 5.81 (s, 2H), 4.16 (q, J = 7.0 Hz, 2H), 1.36 (t, J = 7.0 Hz, 3H). LCMS (Analytical Method A): Rt = 0.92 mins; MS (ESIPos) m/z = 240 (M + H)⁺. |
| 11A | 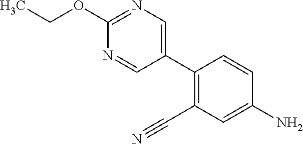 [SMILES: CCOc1ncc(-c2ccc(N)cc2C#N)cn1] | 5-amino-2-(2- ethoxypyrimidin- 5-yl)benzonitrile | ¹H NMR (500 MHz, DMSO-d6) δ [ppm] 8.70 (s, 2H), 7.33 (d, J = 8.4 Hz, 1H), 7.00 (d, J = 2.4 Hz, 1H), 6.95 (dd, J = 8.4, 2.4 Hz, 1H), 5.82 (s, 2H), 4.40 (q, J = 7.0 Hz, 2H), 1.36 (t, J = 7.0 Hz, 3H). LCMS (Analytical Method A): Rt = 1.02 mins; MS (ESIpos) m/z = 240.95 (M + H)⁺. |
| 12A | 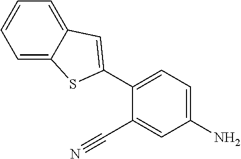 [SMILES: N#Cc1cc(N)ccc1-c1cc2ccccc2s1] | 5-amino-2-(1- benzothiophen- 2-yl)benzonitrile | ¹H NMR (500 MHz, DMSO-d6) δ [ppm] 5.96 (s, 2H), 6.95 (dd, 1H), 7.02 (d, 1H), 7.40 (m, 2H), 7.47 (d, 1H), 7.67 (s, 1H), 7.88 (d, 1H), 7.98 (d, 1H). LCMS (Analytical Method A) Rt = 1.21 min, MS (ESIpos): m/z = 250.9 (M + H)⁺. |
| 13A | 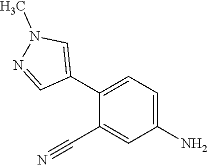 | 5-amino-2-(1- methyl-1H- pyrazol-4- yl)benzonitrile | ¹H NMR (500 MHz, Chloroform-d) δ [ppm] 7.71 (d, J = 8.5 Hz, 1H), 7.40 (d, J = 2.3 Hz, 1H), 6.95 (d, J = 2.5 Hz, 1H), 6.89 (dd, J = 8.5, 2.6 Hz, 1H), 6.80 (d, J = 2.3 Hz, 1H), 3.95 (s, 3H), 3.88 (s, 2H). LCMS (Analytical Method A) Rt = 1.01 min, MS (ESIPos): m/z = 198.9 (M + H)⁺. |
| 14A | 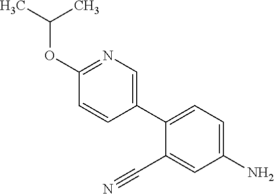 [SMILES: CC(C)Oc1ccc(-c2ccc(N)cc2C#N)cn1] | 5-amino-2-(6- isopropoxy- pyridin-3-yl) benzonitrile | ¹H NMR (500 MHz, DMSO-d6) δ [ppm] 1.31 (d, J = 6.2 Hz, 6H), 5.28 (hept, J = 6.2 Hz, 1H), 5.71 (s, 2H), 6.82 (dd, J = 8.6, 0.6 Hz, 1H), 6.92 (dd, J = 8.5, 2.5 Hz, 1H), 6.97 (d, J = 2.4 Hz, 1H), 7.26 (d, J = 8.4 Hz, 1H), 7.78 (dd, J = 8.6, 2.6 Hz, 1H), 8.22 (dd, J = 2.6, 0.6 Hz, 1H). LCMS (Analytical Method A): Rt = 1.14 mins, MS (ESIPos): m/z = 254.05 (M + H)⁺. |
| 15A | 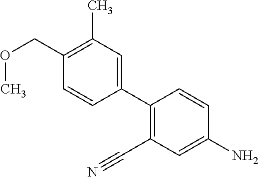 [SMILES: COCc1ccc(-c2ccc(N)cc2C#N)cc1C] | 4-amino-4′- (methoxymethyl)- 3′-methylbiphenyl- 2-carbonitrile | ¹H NMR (500 MHz, DMSO-d6) δ [ppm] 2.31 (s, 3H), 3.34 (s, 3H), 4.44 (s, 2H), 5.68 (s, 2H), 6.91 (dd, J = 8.5, 2.5 Hz, 1H), 6.95 (d, J = 2.4 Hz, 1H), 7.23- 7.28 (m, 3H), 7.35 (d, J = 7.9 Hz, 1H). LCMS (Analytical Method A): Rt = 1.12 mins, MS (ESIPos): m/z = 253.00 (M + H)⁺. |
| 16A | 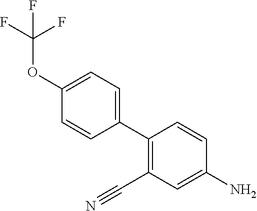 [SMILES: N#Cc1cc(N)ccc1-c1ccc(OC(F)(F)F)cc1] | 4-amino-4′- (trifluoromethoxy) biphenyl-2- carbonitrile | ¹H NMR (500 MHz, DMSO-d6) δ [ppm] 5.78 (s, 2H), 6.84-7.04 (m, 2H), 7.29 (d, J = 8.5 Hz, 1H), 7.45 (d, J = 8.1 Hz, 2H), 7.59 (d, J = 8.6 Hz, 2H). LCMS (Analytical Method A): Rt = 1.2 mins, MS (ESIPos): m/z = 279 (M + H)⁺. |

### Intermediate 17A: 3′,4′-dimethoxy-2-(1H-tetrazol-5-yl)biphenyl-4-amine

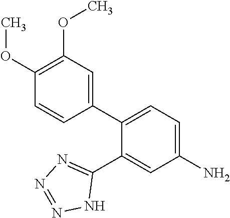 [SMILES: COc1ccc(-c2ccc(N)cc2-c2nnn[nH]2)cc1OC]

4-Amino-3′,4′-dimethoxybiphenyl-2-carbonitrile (12.9 g, 50.7 mmol) was dissolved in toluene (400 mL) and azidotrimethylsilane (26.9 mL, 203 mmol) and di-n-butyl tin oxide (18.9 g, 76.1 mmol) were added at RT. The resulting dark brown mixture was heated to 130° C. (bath temperature) for 14 h. The mixture was cooled and diluted with 250 mL methanol and 100 mL water. The mixture was extracted 3 times with ethyl acetate, the combined organic layers washed with water, brine, dried (Na₂SO₄), filtered and concentrated in vacuo. The residue was purified by Biotage Isolera™ chromatography (silica gel, eluting with DCM/MeOH, 95:5 to 78:22) to yield 13.6 g (90% yield) of the desired product as a light brown foam.
¹-NMR (400 MHz, DMSO-d6) δ [ppm] 3.55 (s, 3H), 3.71 (s, 3H), 6.47 (d, 1H), 6.75 (d, 1H), 6.81−6.83 (m, 2H), 7.24 (d, 1H).
LCMS (method 1): Rₜ=0.62, MS (ESIpos) m/z=298 (M+H)⁺.
In analogy to the procedure described for Intermediate 17A, the following intermediates were prepared using the corresponding nitriles as starting materials.

| Int. | Structure | Name | Analytical Data |
|---|---|---|---|
| 18A | 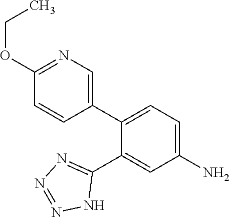 [SMILES: CCOc1ccc(-c2ccc(N)cc2-c2nnn[nH]2)cn1] | 4-(6- ethoxypyridin- 3-yl)-3-(1H- tetrazol-5- yl)aniline | ¹H NMR (400 MHz, DMSO-d6) δ [ppm] 1.30 (t, 3H), 4.26 (q, 2H), 6.64 (d, 1H), 6.81-6.84 (m, 2H), 7.19-7.22 (m, 2H), 7.80 (d, 1H). LCMS (method 1): Rₜ = 0.68, MS (ESIpos) m/z = 283 (M + H)⁺. |
| 19A | 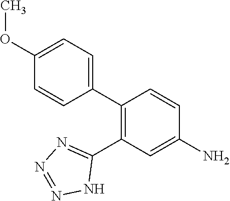 [SMILES: COc1ccc(-c2ccc(N)cc2-c2nnn[nH]2)cc1] | 4′-methoxy-2- (1H-tetrazol-5- yl)biphenyl-4- amine | ¹H NMR (500 MHz, DMSO-d6) δ [ppm] 7.17 (d, J = 8.3 Hz, 1H), 6.89- 6.85 (m, 2H), 6.83-6.77 (m, 3H), 6.75 (d, J = 2.4 Hz, 1H), 5.47 (s, 2H), 3.71 (s, 3H). LCMS (Analytical Method A): Rt = 0.89 mins, MS (ESIpos) m/z = 268 (M + H)⁺. |
| 20A | 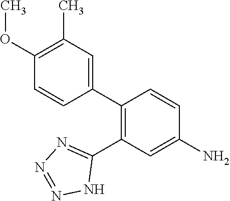 [SMILES: COc1ccc(-c2ccc(N)cc2-c2nnn[nH]2)cc1C] | 4′-methoxy-3′- methyl-2-(1H- tetrazol-5- yl)biphenyl-4- amine | ¹H NMR (500 MHz, DMSO-d6) δ [ppm] 2.05 (s, 3H), 3.73 (s, 3H), 5.44 (s, 2H), 6.66 (dd, J = 8.4, 2.3 Hz, 1H), 6.72- 6.83 (m, 4H), 7.16 (d, J = 8.3 Hz, 1H). LCMS (Analytical Method A): Rt = 0.99 mins, MS (ESIpos) m/z = 282 (M + H)⁺. |
| 21A | 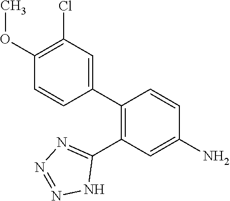 [SMILES: COc1ccc(-c2ccc(N)cc2-c2nnn[nH]2)cc1Cl] | 3′-chloro-4′- methoxy-2-(1H- tetrazol-5- yl)biphenyl-4- amine | ¹H NMR (250 MHz, Methanol-d4) δ [ppm] 7.26 (m, 1H), 7.10 (d, J = 2.1 Hz, 1H), 6.99-6.91 (m, 3H), 6.86 (m, 1H), 3.86 (s, 3H). LCMS (Analytical Method A): Rₜ = 0.97 min; MS (ESIpos) m/z 302 (M + H)⁺. |
| 22A | 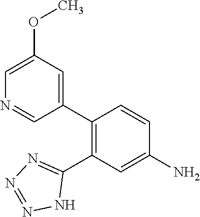 [SMILES: COc1cncc(-c2ccc(N)cc2-c2nnn[nH]2)c1] | 4-(5- methoxypyridin- 3-yl)-3-(1H- tetrazol-5- yl)aniline | ¹H NMR (500 MHz, DMSO-d6) δ [ppm] 8.12 (d, J = 2.8 Hz, 1H), 7.76 (d, J = 1.8 Hz, 1H), 7.30 (d, J = 8.3 Hz, 1H), 6.92 (m, 1H), 6.89-6.80 (m, 2H), 5.67 (s, 2H), 3.72 (s, 3H). |
| 23A | 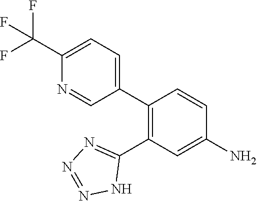 [SMILES: Nc1ccc(-c2ccc(C(F)(F)F)nc2)c(-c2nnn[nH]2)c1] | 3-(1H-tetrazol- 5-yl)-4-[6- (trifluoromethyl) pyridine-3-yl] aniline | ¹H NMR (500 MHz, DMSO-d6) δ [ppm] 5.79 (s, 2H), 6.88 (m, 1H), 6.92 (s, 1H), 7.33 (d, J = 8.4 Hz, 1H), 7.60 (m, 1H), 7.77 (d, J = 8.1 Hz, 1H), 8.40 (d, J = 2.1 Hz, 1H). LCMS (Analytical Method A): Rₜ = 0.97 min; MS (ESIpos) m/z 307 (M + H)⁺. |
| 24A | 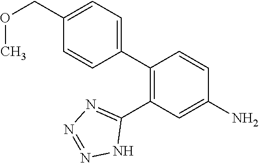 [SMILES: COCc1ccc(-c2ccc(N)cc2-c2nnn[nH]2)cc1] | 4′-(methoxy- methyl)-2- (1H-tetrazol- 5-yl)biphenyl-4- amine | ¹H NMR (250 MHz, Methanol-d4) δ [ppm] 7.32-7.20 (m, 3H), 7.06-6.92 (m, 4H), 4.43 (s, 2H), 3.37 (s, 3H). LCMS (Analytical Method A): Rₜ = 0.89 min; MS (ESIpos) m/z 282 (M + H)⁺. |
| 25A | 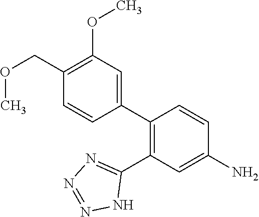 [SMILES: COCc1ccc(-c2ccc(N)cc2-c2nnn[nH]2)cc1OC] | 4′-ethoxy-3′- methoxy-2-(1H- tetrazol-5- yl)biphenyl-4- amine | ¹H NMR (500 MHz, DMSO-d6) δ [ppm] 1.29 (t, J = 6.9 Hz, 3H), 3.55 (s, 3H), 3.95 (q, J = 6.8 Hz, 2H), 5.75 (s, 2H), 6.38-6.51 (m, 2H), 6.69-6.88 (m, 3H), 7.23 (d, J = 8.3 Hz, 1H). LCMS (Analytical Method A): Rₜ = 1.12 min; MS (ESIpos) m/z 312 (M + H)⁺. |
| 26A | 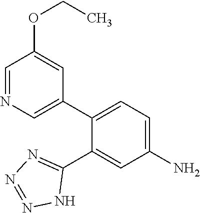 [SMILES: CCOc1cncc(-c2ccc(N)cc2-c2nnn[nH]2)c1] | 4-(5- ethoxypyridin- 3-yl)-3-(1H- tetrazol-5- yl)aniline | ¹H NMR (500 MHz, DMSO-d6) δ [ppm] 8.09 (d, J = 2.7 Hz, 1H), 7.75 (d, J = 1.7 Hz, 1H), 7.28 (d, J = 8.2 Hz, 1H), 6.90-6.87 (m, 1H), 6.87-6.82 (m, 2H), 3.96 (q, J = 7.0 Hz, 2H), 1.28 (t, J = 7.0 Hz, 3H). LCMS (Analytical Method A): Rt = 0.98 mins; MS (ESIPos) m/z = 282 (M + H)⁺. |
| 27A | 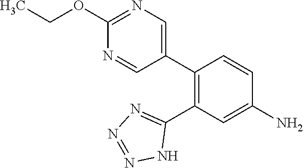 [SMILES: CCOc1ncc(-c2ccc(N)cc2-c2nnn[nH]2)cn1] | 4-(2-ethoxy- pyrimidin- 5-yl)-3-(1H- tetrazol-5- yl)aniline | ¹H NMR (500 MHz, DMSO-d6) δ [ppm] 8.47-8.43 (m, 2H), 6.96 (d, J = 8.3 Hz, 1H), 6.76-6.69 (m, 2H), 3.77 (s, 3H), 3.61 (s, 3H). LCMS (Analytical Method A): Rt = 0.88 mins; MS (ESIpos) m/z = 283.95 (M + H)⁺. |
| 28A | 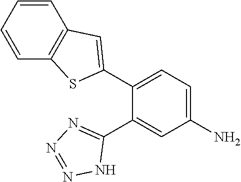 [SMILES: Nc1ccc(-c2cc3ccccc3s2)c(-c2nnn[nH]2)c1] | 4-(1- benzothiophen- 2-yl)-3-(1H- 1,2,3,4-tetrazol- 5-yl)aniline | ¹H NMR (500 MHz, DMSO-d6) δ [ppm] 5.79 (s, 2H), 6.77 (d, J = 2.2 Hz, 1H), 6.86 (d, J = 8.5 Hz, 1H), 6.89 (s, 1H), 7.22-7.35 (m, 2H), 7.45 (d, J = 8.4 Hz, 1H), 7.69 (d, J = 8.0 Hz, 1H), 7.83 (d, J = 7.8 Hz, 1H). LCMS (Analytical Method D) Rt = 3.67 min, MS (ESIpos): m/z = 293.9 (M + H)⁺. |
| 29A | 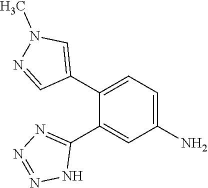 [SMILES: Cn1cc(-c2ccc(N)cc2-c2nnn[nH]2)cn1] | 4-(1-methyl-1H- pyrazol-4-yl)-3- (1H-tetrazol-5- yl)aniline | ¹H NMR (500 MHz, Methanol-d4) δ [ppm] 7.52-7.47 (m, 1H), 7.42 (d, J = 2.2 Hz, 1H), 6.94-6.90 (m, 2H), 5.76 (d, J = 2.3 Hz, 1H), 3.81 (s, 3H). LCMS (Analytical Method A): Rt = 0.70 min, MS (ESIpos): no ionization observed |
| 30A | 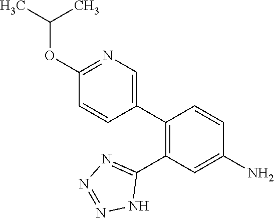 [SMILES: CC(C)Oc1ccc(-c2ccc(N)cc2-c2nnn[nH]2)cn1] | 4-(6- isopropoxy- pyridin-3-yl)- 3-(1H-tetrazol- 5-yl)aniline | ¹H NMR (500 MHz, DMSO-d6) δ [ppm] 1.26 (d, J = 6.2 Hz, 6H), 5.19 (hept, J = 6.2 Hz, 1H), 5.57 (s, 2H), 6.59 (d, J = 8.5 Hz, 1H), 6.77-6.86 (m, 2H), 7.11-7.25 (m, 2H), 7.79 (d, J = 2.5 Hz, 1H). LCMS (Analytical Method A): Rt = 0.94 mins, MS (ESIPos): m/z = 297.00 (M + H)⁺. |
| 31A | 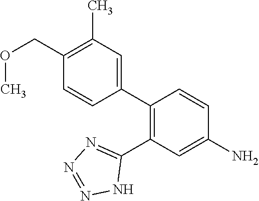 [SMILES: COCc1ccc(-c2ccc(N)cc2-c2nnn[nH]2)cc1C] | 4′-(methoxy- methyl)-3′- methyl-2-(1H- tetrazol-5-yl) biphenyl-4- amine | ¹H NMR (250 MHz, DMSO-d6) δ [ppm] 2.17 (s, 3H), 3.29 (s, 3H), 4.34 (s, 2H), 5.51 (s, 2H), 6.65 (dd, J = 7.7, 1.7 Hz, 1H), 6.71-6.91 (m, 3H), 7.09 (d, J = 7.8 Hz, 1H), 7.20 (d, J = 8.3 Hz, 1H). LCMS (Analytical Method A): Rt = 0.94 mins, MS (ESIPos): m/z = 296.0 (M + H)⁺. |
| 32A | 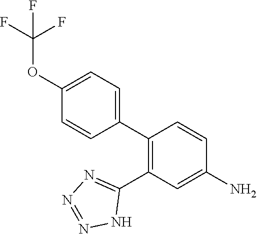 [SMILES: Nc1ccc(-c2ccc(OC(F)(F)F)cc2)c(-c2nnn[nH]2)c1] | 2-(1H-tetrazol- 5-yl)-4′-(trifluoro- methoxy)biphenyl- 4-amine | ¹H NMR (500 MHz, Methanol-d4) δ [ppm] 6.91-7.00 (m, 2H), 7.07-7.13 (m, 2H), 7.15 (d, J = 8.3 Hz, 2H), 7.28 (d, J = 8.2 Hz, 1H). LCMS (Analytical Method A): Rt = 1.05 mins, MS (ESIPos): m/z = 322 (M + H)⁺. |

### Intermediate 33A: 2-(6-Ethoxypyridin-3-yl)-4-methyl-5-nitrobenzonitrile

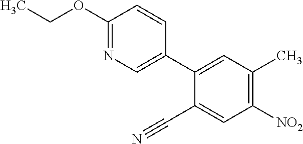 [SMILES: CCOc1ccc(-c2cc(C)c([N+](=O)[O-])cc2C#N)cn1]

A solution of 2-bromo-4-methyl-5-nitrobenzonitrile (2 g, 8.3 mmol), 2-ethoxy-5-(4,4,5,5-tetramethyl-1,3,2-dioxaborolan-2-yl)pyridine (2.07 g, 8.3 mmol) and potassium carbonate (3.78 g, 27 mmol) in dimethoxyethane (20 mL) and water (8 mL) was degassed with a stream of nitrogen for 10 mins. Dichlorobis(triphenylphosphine)palladium(II) (58 mg, 0.08 mmol) was added, and the reaction heated at 100° C. for 2 h under nitrogen. The reaction mixture was dissolved into ethyl acetate (250 mL) and washed with water (30 mL) then brine (30 mL), dried over anhydrous Na₂SO₄, filtered and concentrated under reduced pressure. The crude residue was triturated from dichloromethane/heptane, and the precipitate collected by vacuum filtration. The mother liqueur was concentrated and the residue was purified by Biotage Isolera™ chromatography (silica gel, eluting with heptanes—EtOAc, 100:0 to 0:100). The batches were combined to afford 1.71 g (68% yield) of the title compound as an off-white solid. ¹H NMR (500 MHz, DMSO-d6) δ [ppm] 1.36 (t, J=7.0 Hz, 3H), 2.64 (s, 3H), 4.40 (q, J=7.0 Hz, 2H), 7.00 (d, J=8.6 Hz, 1H), 7.86 (s, 1H), 8.03 (dd, J=8.6, 2.6 Hz, 1H), 8.46 (d, J=2.6 Hz, 1H),8.64 (s, 1H).
LCMS (Analytical Method A): Rt=1.25 mins; MS (ESIPos) m/z=283.95 (M+H)⁺.

In analogy to the procedure described for Intermediate 33A, the following intermediates were prepared using the corresponding boronic acid as starting material.

| Int. | Structure | Name | Analytical Data |
|---|---|---|---|
| 34A | 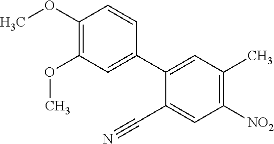 [SMILES: COc1ccc(-c2cc(C)c([N+](=O)[O-])cc2C#N)cc1OC] | 3′,4′- Dimethoxy-5- methyl-4- nitrobiphenyl-2- carbonitrile | ¹H NMR (500 MHz, DMSO-d6) δ [ppm] 2.65 (s, 3H), 3.84 (s, 6H), 7.15 (d, J = 8.4 Hz, 1H), 7.19-7.32 (m, 2H), 7.83 (s, 1H), 8.59 (s, 1H). LCMS (Analytical Method A): Rt = 1.19 mins. |
| 35A | 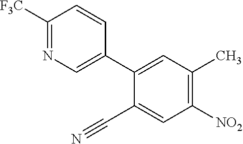 [SMILES: Cc1cc(-c2ccc(C(F)(F)F)nc2)c(C#N)cc1[N+](=O)[O-]] | 4-Methyl-5- nitro-2-[6- (trifluoromethyl) pyridin-3-yl] benzonitrile | ¹H NMR (500 MHz, Chloroform-d) δ [ppm] 8.90 (d, J = 2.0 Hz, 1H), 8.47 (s, 1H), 8.15 (dd, J = 8.1, 2.2 Hz, 1H), 7.88 (d, J = 8.1 Hz, 1H), 7.56 (s, 1H), 2.78 (s, 3H). LCMS (Analytical Method A): Rt = 1.20 mins; MS (ESIPos) m/z = 307.95 (M + H)⁺. |
| 36A | 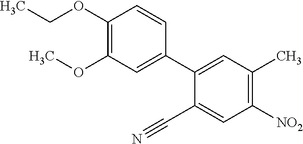 [SMILES: CCOc1ccc(-c2cc(C)c([N+](=O)[O-])cc2C#N)cc1OC] | 4′-Ethoxy-3′- methoxy-5- methyl-4- nitrobiphenyl-2- carbonitrile | ¹H NMR (500 MHz, DMSO-d6) δ [ppm] 1.37 (t, J = 7.0 Hz, 3H), 2.64 (s, 3H), 3.84 (s, 3H), 4.10 (q, J = 7.0 Hz, 2H), 7.13 (d, J = 8.4 Hz, 1H), 7.22 (dd, J = 8.3, 2.2 Hz, 1H), 7.27 (d, J = 2.2 Hz, 1H), 7.82 (s, 1H), 8.58 (s, 1H). LCMS (Analytical Method A): Rt = 1.26 mins. |
| 37A | 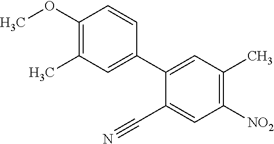 [SMILES: COc1ccc(-c2cc(C)c([N+](=O)[O-])cc2C#N)cc1C] | 4′-Methoxy-3′,5- dimethyl-4- nitrobiphenyl-2- carbonitrile | ¹H NMR (500 MHz, Chloroform-d) δ [ppm] 8.40 (s, 1H), 7.48 (s, 1H), 7.44 (dd, J = 8.4, 2.4 Hz, 1H), 7.35 (d, J = 2.3 Hz, 1H), 6.96 (d, J = 8.5 Hz, 1H), 3.91 (s, 3H), 2.73 (s, 3H), 2.30 (s, 3H). LCMS (Analytical Method A): Rt = 1.31 min. |
| 38A | 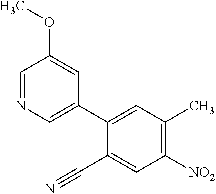 [SMILES: COc1cncc(-c2cc(C)c([N+](=O)[O-])cc2C#N)c1] | 2-(5- methoxypyridin- 3-yl)-4-methyl- 5-nitro- benzonitrile | ¹H NMR (500 MHz, Chloroform-d) δ 8.46 (d, J = 2.8 Hz, 1H), 8.44 (s, 1H), 8.37 (d, J = 1.8 Hz, 1H), 7.55 (s, 1H), 7.46-7.40 (m, 1H), 3.95 (s, 3H), 2.76 (s, 3H). LCMS (Analytical Method A): Rt = 1.08 min, m/z = 269.9 [M + H]⁺ |

### Intermediate 39A: 2-Ethoxy-5-[5-methyl-4-nitro-2-(1H-tetrazol-5-yl)phenyl]pyridine

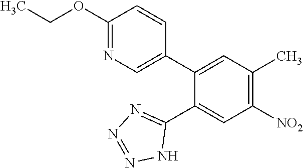 [SMILES: CCOc1ccc(-c2cc(C)c([N+](=O)[O-])cc2-c2nnn[nH]2)cn1]

2-(6-Ethoxypyridin-3-yl)-4-methyl-5-nitrobenzonitrile (1.71 g, 5.6 mmol) was split between two sealed tubes. To both tubes p-xylene (8 mL), di-n-butyltin oxide (700 mg, 2.8 mmol) and azidotrimethylsilane (1.12 mL, 8.4 mmol) were added. The resulting mixtures were heated in a sealed tube at 130° C. for 16 h. The reaction was cooled to RT, MeOH (10 mL) was added to both tubes and the mixture stirred at RT for 1 hour then reaction mixtures combined and concentrated at reduced pressure. The residue was purified by Biotage Isolera™ chromatography (silica gel, eluting with DCM/MeOH, 100:0 to 85:15) to afford 1.41 g (72% yield) of the title compound as a yellow solid.
¹H NMR (500 MHz, DMSO-d6) δ [ppm] 1.32 (t, J=7.0 Hz, 3H), 2.66 (s, 3H), 4.32 (q, J=7.0 Hz, 2H), 6.77 (d, J=8.6 Hz, 1H), 7.44 (dd, J=8.6, 2.4 Hz, 1H), 7.75 (s, 1H), 8.03 (d, J=2.4 Hz, 1H), 8.41 (s, 1H).
LCMS (Analytical Method A): Rt=1.10 mins; MS (ESIPos) m/z=327.10 (M+H)⁺.
In analogy to the procedure described for Intermediate 39A, the following intermediates were prepared using the corresponding nitrile as starting material.

| Int. | Structure | Name | Analytical Data |
|---|---|---|---|
| 40A | 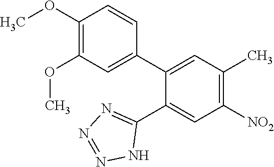 [SMILES: COc1ccc(-c2cc(C)c([N+](=O)[O-])cc2-c2nnn[nH]2)cc1OC] | 5-(3′,4′- Dimethoxy-5- methyl-4- nitrobiphenyl-2- yl)-1H-tetrazole | ¹H NMR (500 MHz, DMSO-d6) δ [ppm] 2.67 (s, 3H), 3.61 (s, 3H), 3.76 (s, 3H), 6.63-6.72 (m, 2H), 6.93 (d, J = 8.2 Hz, 1H), 7.74 (s, 1H), 8.30 (s, 1H). LCMS (Analytical Method A): Rt = 1.06 mins; MS (ESIPos) m/z = 342.1 (M + H)⁺. |
| 41A | 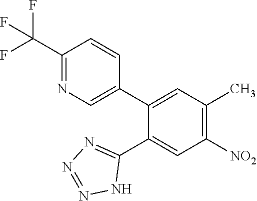 [SMILES: Cc1cc(-c2ccc(C(F)(F)F)nc2)c(-c2nnn[nH]2)cc1[N+](=O)[O-]] | 5-[5-Methyl-4- nitro-2-(1H- tetrazol-5- yl)phenyl]-2- (trifluoromethyl) pyridine | ¹H NMR (500 MHz, Methanol-d4) δ [ppm] 8.56 (d, J = 1.7 Hz, 1H), 8.52 (s, 1H), 7.90 (dd, J = 8.1, 1.7 Hz, 1H), 7.83 (d, J = 8.1 Hz, 1H), 7.71 (s, 1H), 2.74 (s, 3H). LCMS (Analytical Method A): Rt = 1.12 min; MS (ESIPos) m/z = 351.1 (M + H)⁺. |
| 42A | 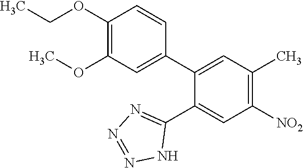 [SMILES: CCOc1ccc(-c2cc(C)c([N+](=O)[O-])cc2-c2nnn[nH]2)cc1OC] | 5-(4′-Ethoxy-3′- methoxy-5- methyl-4- nitrobiphenyl-2- yl)-1H-tetrazole | ¹H NMR (500 MHz, DMSO-d6) δ [ppm] 1.32 (t, J = 6.9 Hz, 3H), 2.67 (s, 3H), 3.61 (s, 3H), 4.01 (q, J = 6.9 Hz, 2H), 6.61-6.74 (m, 2H), 6.91 (d, J = 8.2 Hz, 1H), 7.74 (s, 1H), 8.29 (s, 1H). LCMS (Analytical Method A): Rt = 1.12 mins. |
| 43A | 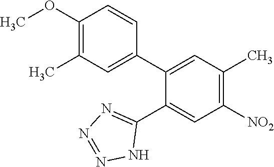 [SMILES: COc1ccc(-c2cc(C)c([N+](=O)[O-])cc2-c2nnn[nH]2)cc1C] | 5-(4′-Methoxy- 3′,5-dimethyl-4- nitrobiphenyl-2- yl)-1H-tetrazole | ¹H NMR (500 MHz, Chloroform-d) δ [ppm] 8.65 (s, 1H), 7.38 (s, 1H), 7.01- 6.75 (m, 3H), 3.83 (s, 3H), 2.68 (s, 3H), 2.14 (s, 3H). LCMS (Analytical Method A): Rt = 1.16 min; MS (ESIPos) m/z = 326.0 (M + H)⁺. |
| 44A | 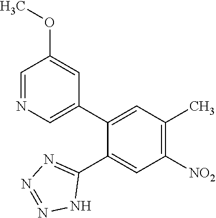 [SMILES: COc1cncc(-c2cc(C)c([N+](=O)[O-])cc2-c2nnn[nH]2)c1] | 3-methoxy-5-[5- methyl-4-nitro- 2-(1H-tetrazol- 5-yl)phenyl] pyridine | ¹H NMR (500 MHz, Methanol-d4) δ 8.30 (s, 1H), 8.15 (d, J = 2.6 Hz, 1H), 7.94 (s, 1H), 7.65 (s, 1H), 7.17 (s, 1H), 3.77 (s, 3H), 2.70 (s, 3H). LCMS (Analytical Method A): Rt = 0.93 min, m/z = 313.1 [M + H]⁺. |

### Intermediate 45A: 4-(6-Ethoxypyridin-3-yl)-2-methyl-5-(1H-tetrazol-5-yl)aniline

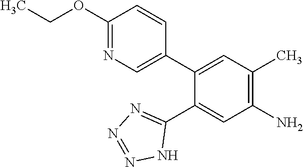 [SMILES: CCOc1ccc(-c2cc(C)c(N)cc2-c2nnn[nH]2)cn1]

To a solution of 2-ethoxy-5-[5-methyl-4-nitro-2-(1H-tetrazol-5-yl)phenyl]pyridine (1.41 g, 4.02 mmol) in ethanol (100 mL) was added 10% palladium on carbon (436 mg, 0.4 mmol) and the reaction stirred under an atmosphere of hydrogen for 4 h. The reaction was filtered through Celite® and concentrated under reduced pressure then triturated in DCM/heptane to afford 1.3 g (91% yield) of the title compound.
¹H NMR (500 MHz, DMSO-d6) δ [ppm] 1.29 (t, J=7.0 Hz, 3H), 2.17 (s, 3H), 4.26 (q, J=7.0 Hz, 2H), 5.31 (s, 2H), 6.64 (d, J=8.5 Hz, 1H), 6.87 (s, 1H), 7.11 (s, 1H), 7.22 (dd, J=8.5, 2.5 Hz, 1H), 7.80 (d, J=2.2 Hz, 1H).
LCMS (Analytical Method A): Rt=0.95 mins; MS (ESIPos) m/z=297.1 (M+H)⁺.
In analogy to the procedure described for Intermediate 45A, the following intermediates were prepared using the corresponding nitro compound as starting material.

| Int. | Structure | Name | Analytical Data |
|---|---|---|---|
| 46A | 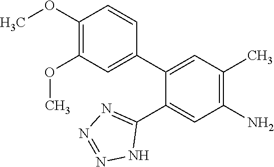 | 3′,4′- Dimethoxy-5- methyl-2-(1H- tetrazol-5- yl)biphenyl-4- amine | ¹H NMR (250 MHz, DMSO-d6) δ [ppm] 2.17 (s, 3H), 3.56 (s, 3H), 3.71 (s, 3H), 5.23 (s, 2H), 6.41-6.55 (m, 2H), 6.76-6.86 (m, 2H), 7.14 (s, 1H). LCMS (Analytical Method A): Rt = 0.91 mins; MS (ESINeg) m/z = 310.15 (M − H)⁻. |
| 47A | 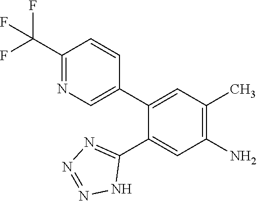 [SMILES: Cc1cc(-c2ccc(C(F)(F)F)nc2)c(-c2nnn[nH]2)cc1N] | 2-Methyl-5-(1H- tetrazol-5-yl)- 4-[6-(trifluoro- methyl) pyridin-3-yl] aniline | ¹H NMR (500 MHz, Methanol-d4) δ 8.36 (s, 1H), 7.71-7.63 (m, 2H), 7.24 (s, 1H), 7.04 (s, 1H), 2.28 (s, 3H). LCMS (Analytical Method A): Rt = 3.54 min; MS (ESIPos) m/z = 321.1 (M + H)⁺. |
| 48A | 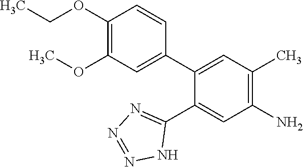 [SMILES: CCOc1ccc(-c2cc(C)c(N)cc2-c2nnn[nH]2)cc1OC] | 4′-Ethoxy-3′- methoxy-5- methyl-2-(1H- tetrazol-5-yl) biphenyl-4- amine | ¹H NMR (500 MHz, DMSO-d6) δ [ppm] 1.29 (t, J = 6.9 Hz, 3H), 3.55 (s, 3H), 3.95 (q, J = 6.8 Hz, 2H), 5.75 (s, 2H), 6.38-6.51 (m, 2H), 6.69-6.88 (m, 3H), 7.23 (d, J = 8.3 Hz, 1H). LCMS (Analytical Method A): Rt = 0.98 mins; MS (ESIPos) m/z = 326.0 (M + H)⁺. |
| 49A | 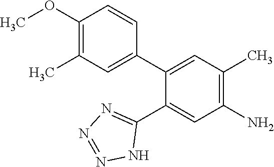 | 4′-Methoxy-3′,5- dimethyl-2-(1H- tetrazol-5-yl) biphenyl-4- amine | ¹H NMR (500 MHz, Methanol-d4) δ [ppm] 7.17 (s, 1H), 6.95 (s, 1H), 6.84 (s, 1H), 6.82-6.74 (m, 2H), 3.81 (s, 3H), 2.28 (s, 3H), 2.12 (s, 3H). LCMS (Analytical Method A): Rt = 1.03 min; MS (ESIPos) m/z = 296.05 (M + H)⁺. |
| 50A | 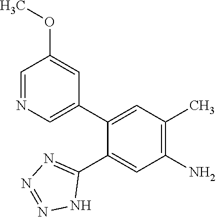 [SMILES: COc1cncc(-c2cc(C)c(N)cc2-c2nnn[nH]2)c1] | 4-(5-methoxy- pyridin-3-yl)- 2-methyl-5- (1H-tetrazol- 5-yl)aniline | ¹H NMR (250 MHz, Methanol-d4) δ 8.00 (d, J = 2.8 Hz, 1H), 7.82 (d, J = 1.7 Hz, 1H), 7.19 (s, 1H), 7.00 (dd, J = 2.7, 1.8 Hz, 1H), 6.97 (s, 1H), 3.72 (s, 3H), 2.27 (s, 3H). LCMS (Analytical Method A): Rt = 0.68 min, m/z = 283 [M + H]⁺. |

Intermediate 51A: 5-fluoro-3′,4′-dimethoxy-4-nitrobiphenyl-2-carbonitrile

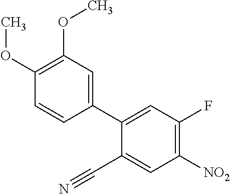 [SMILES: COc1ccc(-c2cc(F)c([N+](=O)[O-])cc2C#N)cc1OC]

Water (37 mL) and 1,2-dimethoxyethane (74 mL) were degassed with a stream of argon for 15 mins. Then 2-bromo-4-fluoro-5-nitrobenzonitrile (5.00 g, 20.4 mmol), 3,4-dimethylbenzeneboronic acid (3.71 g, 20.4 mmol), potassium carbonate (9.31 g, 67.3 mmol) and dichlorobis(triphenylphosphine) palladium(II) (172 mg, 0.245 mmol) were added, and the reaction heated at 90° C. for 5 h. The mixture was cooled and put into water. Ethyl acetate was added and the layers separated. The aqueous layer was extracted 3× with ethyl acetate. The combined organic layers were washed with brine, dried with sodium sulfate and concentrated under reduced pressure. The residue was purified by Biotage Isolera™ chromatography (silica gel, eluting with hexane/ethyl acetate, 100:0 to 1:1) to yield 2.96 g (38% yield) of the desired product.
¹H NMR (400 MHz, DMSO-d) δ [ppm] 3.34 (s, 3H), 3.86 (s, 3H), 7.18 (d, 1H), 7.30-7.33 (m, 2H), 8.00 (d, 1H), 8.84 (d, 1H);
LCMS (method 1): Rt=1.09, MS (ESIpos) m/z=303 (M+H)⁺.
In analogy to the procedure described for Intermediate 51A, the following intermediates were prepared using the corresponding boronic acid as starting material.

| Int. | Structure | Name | Analytical Data |
|---|---|---|---|
| 52A | 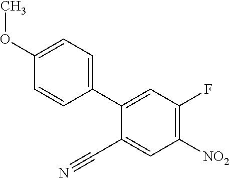 [SMILES: COc1ccc(-c2cc(F)c([N+](=O)[O-])cc2C#N)cc1] | 5-fluoro-4′- methoxy-4- nitrobiphenyl-2- carbonitrile | ¹H NMR (400 MHz, DMSO-d) δ [ppm] 3.86 (s, 3H), 7.16 (d, 2H), 7.69 (d, 2H), 7.94 (d, 1H), 8.84 (d, 1H). LCMS (method 1): Rt = 1.15; MS (ESIpos) m/z = 273 (M + H)⁺ |
| 53A | 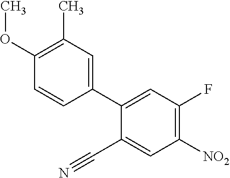 [SMILES: COc1ccc(-c2cc(F)c([N+](=O)[O-])cc2C#N)cc1C] | 5-fluoro-4′- methoxy-3′- methyl-4- nitrobiphenyl-2- carbonitrile | ¹H NMR (400 MHz, DMSO-d) δ [ppm] 2.24 (s, 3H), 3.89 (s, 3H), 7.16 (d, 1H), 7.53 (m, 1H), 7.59 (dd, 1H), 7.92 (d, 1H), 8.83 (d, 1H). LCMS (method 1): Rt = 1.27; MS (ESIpos) m/z = 287 (M + H)⁺ |

### Intermediate 54A: 5-(5-fluoro-3′,4′-dimethoxy-4-nitrobiphenyl-2-yl)-1H-tetrazole

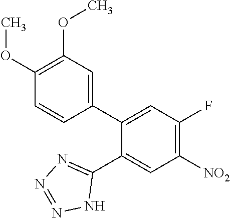 [SMILES: COc1ccc(-c2cc(F)c([N+](=O)[O-])cc2-c2nnn[nH]2)cc1OC]

5-Fluoro-3′,4′-dimethoxy-4-nitrobiphenyl-2-carbonitrile (Intermediate 51A) (710 mg, 2.35 mmol) were dissolved in toluene (25 mL) and azidotrimethylsilane (1.25 mL, 9.37 mmol) and di-n-butyl tin oxide (877 mg, 3.52 mmol) were added at RT. The mixture was heated to 130° C. (bath temperature) for 4 h. The mixture was cooled and diluted with methanol. Silica gel was added and the solvents evaporated under reduced pressure. The residue was purified by Biotage Isolera™ chromatography (silica gel, eluting with DCM/MeOH, 100:0 to 70:30) to yield 720 mg (89% yield) of the desired product as a yellow solid.
¹H NMR (400 MHz, DMSO-d6) δ [ppm] 3.63 (s, 3H), 3.77 (s, 3H), 6.69 (dd, 1H), 6.75 (d, 1H), 6.94 (d, 1H), 7.90 (d, 1H), 8.47 (d, 1H).
LCMS (method 2): Rₜ=0.90; MS (ESIpos) m/z=346 (M+H)⁺.
In analogy to the procedure described for Intermediate 54A, the following intermediates were prepared using the corresponding nitrile as starting material.

| Int. | Structure | Name | Analytical Data |
|---|---|---|---|
| 55A | 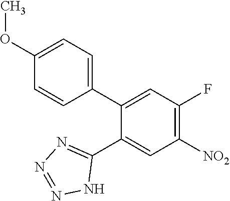 [SMILES: COc1ccc(-c2cc(F)c([N+](=O)[O-])cc2-c2nnn[nH]2)cc1] | 5-(5-fluoro-4′- methoxy-4- nitrobiphenyl-2- yl)-1H-tetrazole | ¹H NMR (400 MHz, DMSO-d6) δ [ppm] 3.77 (s, 3H), 6.90 (d, 2H), 7.12 (d, 2H), 7.72 (d, 1H), 8.39 (d, 1H). LCMS (method 1) Rt = 0.95; MS (ESIpos) m/z = 316 (M + H)⁺ |
| 56A | 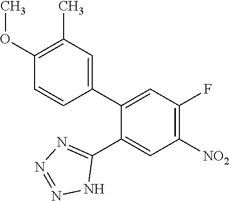 [SMILES: COc1ccc(-c2cc(F)c([N+](=O)[O-])cc2-c2nnn[nH]2)cc1C] | 5-(5-fluoro-4′- methoxy-3′- methyl-4- nitrobiphenyl-2- yl)-1H-tetrazole | ¹H NMR (400 MHz, DMSO-d) δ [ppm] 2.10 (s, 3H), 3.80 (s, 3H), 6.89-6.90 (m, 2H), 7.05 (m, 1H), 7.82 (d, 1H), 8.47 (d, 1H). LCMS (method 1): Rt = 1.06; MS (ESIpos) m/z = 330 (M + H)⁺ |

### Intermediate 57A: 5-fluoro-3′,4′-dimethoxy-2-(1H-tetrazol-5-yl)biphenyl-4-amine

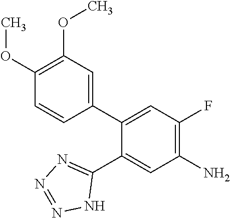 [SMILES: COc1ccc(-c2cc(F)c(N)cc2-c2nnn[nH]2)cc1OC]

5-(5-Fluoro-3′,4′-dimethoxy-4-nitrobiphenyl-2-yl)-1H-tetrazole (Intermediate 54A) (720 mg, 2.09 mmol) was dissolved in ethanol (12 mL) and palladium on carbon (10%, 220 mg) added. The mixture was shaken under hydrogen atmosphere for 3 h. Then the catalyst was filtered off and washed with EtOH and DCM. The filtrate was evaporated to dryness under reduced pressure. The crude product (off white foam, 640 mg, 90% yield) was used without further purification.
¹H NMR (400 MHz, DMSO-d6) δ [ppm] 3.58 (s, 3H), 3.72 (s, 3H), 6.50 (dd, 1H), 6.53 (d, 1H), 6.83 (d, 1H), 6.98 (d, 1H), 7.25 (d, 1H).
LCMS (method 1): Rt=0.74; MS (ESIpos) m/z=316 (M+H)⁺.
In analogy to the procedure described for Intermediate 57A, the following intermediates were prepared using the corresponding nitro compound as starting material.

| Int. | Structure | Name | Analytical Data |
|---|---|---|---|
| 58A | 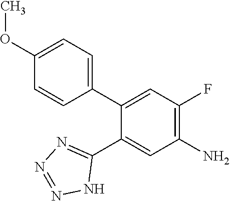 [SMILES: COc1ccc(-c2cc(F)c(N)cc2-c2nnn[nH]2)cc1] | 5-fluoro-4′- methoxy-2-(1H- tetrazol-5- yl)biphenyl-4- amine | ¹H NMR (400 MHz, DMSO-d6) δ [ppm] 3.72 (s, 3H), 6.81 (d, 2H), 6.91 (d, 2H), 6.97 (d, 1H), 7.l7 (d, 1H). LCMS (method 1): Rt = 0.79; MS (ESIpos) m/z = 286 (M + H)⁺. |
| 59A | 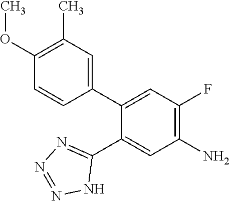 [SMILES: COc1ccc(-c2cc(F)c(N)cc2-c2nnn[nH]2)cc1C] | 5-fluoro-4′- methoxy-3′- methyl-2-(1H- tetrazol-5- yl)biphenyl-4- amine | ¹H NMR (400 MHz, DMSO-d) δ [ppm] 2.06 (s, 3H), 3.75 (s, 3H), 6.69 (dd, 1H), 6.78 (d, 1H), 6.85 (d, 1H), 6.97 (d, 1H), 7.16 (d, 1H). LCMS (method 1): Rt = 0.91; MS (ESIpos) m/z = 300 (M + H)⁺. |

### Intermediate 60A: 2-Bromo-3-fluoro-5-nitrobenzonitrile

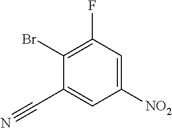 [SMILES: N#Cc1cc([N+](=O)[O-])cc(F)c1Br]

To a solution of 2-bromo-3-fluorobenzonitrile (10.0 g, 50 mmol) in sulfuric acid (22 mL) was added nitric acid (69%, 4.82 mL, 75 mmol) dropwise, maintaining the temperature between 5 and 10° C. The reaction mixture turned red-orange at the end of the addition. The reaction mixture was allowed to warm up to RT and was stirred for 1 hour. The reaction mixture was then poured onto crushed ice. The products were extracted with EtOAc (2×100 mL). The combined organic layers were washed with brine (50 mL), dried over MgSO₄, filtered and concentrated under reduced pressure. The residue was suspended in MeOH (50 mL) and the mixture was briefly heated at reflux. After cooling to RT, a pale yellow precipitate was collected by vacuum filtration, washed with a small amount of MeOH and dried in the vacuum oven. The material was recrystallized from MeOH to afford 1.77 g (14% yield) of the title compound as a yellow powder.

¹H NMR (500 MHz, DMSO-d6) δ [ppm] 8.77 (dd, J=2.5, 1.4 Hz, 1H), 8.65 (dd, J=8.3, 2.5 Hz, 1H).

LCMS (Analytical Method A): Rt=1.11 min, mass ion not observed.

### Intermediate 61A: 6-Fluoro-3′,4′-dimethoxy-4-nitrobiphenyl-2-carbonitrile

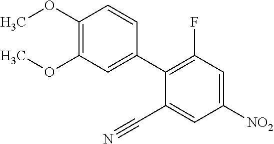 [SMILES: COc1ccc(-c2c(F)cc([N+](=O)[O-])cc2C#N)cc1OC]

A mixture of 2-bromo-3-fluoro-5-nitrobenzonitrile (1.6 g, 6.2 mmol), (3,4-dimethoxyphenyl)boronic acid (1.2 g, 6.8 mmol) and potassium carbonate (2.8 g, 20.5 mmol) in dimethoxyethane (11 mL) and water (8 mL) was heated at reflux under nitrogen atmosphere for 10 minutes. Dichlorobis(triphenyl phosphine) palladium(II) (30 mg, 0.043 mmol) was added to the reaction and the mixture was heated at 100° C. for 2 hours. After cooling to RT, the reaction mixture was diluted with chloroform (100 mL) and water (30 mL). The organic layer was separated the aqueous layer was extracted with chloroform (2×50 mL). The combined organic extracts were washed with brine (30 mL), dried (MgSO4), filtered and concentrated under reduced pressure. The residue was purified by Biotage Isolera™ chromatography (silica gel, eluting with DCM/ethyl acetate, 100:0 to 0:100) to afford 1.71 g (90% yield) of the title compound as an orange solid.
¹H NMR (500 MHz, DMSO-d6) δ [ppm] 8.75-8.69 (m, 1H), 8.58 (dd, J=9.3, 2.2 Hz, 1H), 7.23 (s, 1H), 7.19-7.14 (m, 2H), 3.85 (s, 3H), 3.80 (s, 3H).
LCMS (Analytical Method A): Rt=4.18 mins.
In analogy to the procedure described for Intermediate 61A, the following intermediates were prepared using the corresponding boronic acid as starting material.

| Int. | Structure | Name | Analytical Data |
|---|---|---|---|
| 62A | 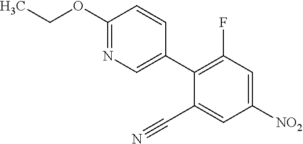 [SMILES: CCOc1ccc(-c2c(F)cc([N+](=O)[O-])cc2C#N)cn1] | 2-(6-ethoxypyridin- 3-yl)-3-fluoro-5- nitrobenzonitrile | ¹H NMR (500 MHz, DMSO-d6) δ [ppm] 8.84-8.74 (m, 1H), 8.64 (dd, J = 9.3, 2.2 Hz, 1H), 8.46-8.38 (m, 1H), 7.98 (ddd, J = 8.6, 2.4, 1.2 Hz, 1H), 7.03 (d, J = 8.6 Hz, 1H), 4.40 (q, J = 7.0 Hz, 2H), 1.36 (t, J = 7.0 Hz, 3H). LCMS (Analytical Method A): Rt = 1.24 mins; MS (ESIpos) m/z = 287.9 (M + H)⁺. |
| 63A | 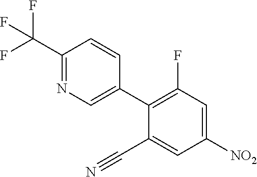 [SMILES: N#Cc1cc([N+](=O)[O-])cc(F)c1-c1ccc(C(F)(F)F)nc1] | 3-fluoro-5-nitro-2-[6- (trifluoromethyl)pyridin- 3-yl]benzonitrile | ¹H NMR (500 MHz, DMSO-d6) δ 9.06 (s, 1H), 8.93-8.85 (m, 1H), 8.75 (dd, J = 9.2, 2.2 Hz, 1H), 8.51-8.40 (m, 1H), 8.22 (d, J = 8.2 Hz, 1H). LCMS (Analytical Method A): Rt = 1.21 mins; MS (ESIpos) m/z = 311.95 (M + H)⁺. |

### Intermediate 64A: 5-(6-Fluoro-3′,4′-dimethoxy-4-nitrobiphenyl-2-yl)-1H-tetrazole

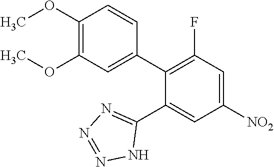 [SMILES: COc1ccc(-c2c(F)cc([N+](=O)[O-])cc2-c2nnn[nH]2)cc1OC]

An ACE pressure tube was loaded with 6-fluoro-3′,4′-dimethoxy-4-nitrobiphenyl-2-carbonitrile (1.7 g, 5.6 mmol), p-xylene (10 mL), di-n-butyltin oxide (1.4 g, 5.5 mmol) and azidotrimethylsilane (850 μL, 6.4 mmol). The pressure tube was sealed and heated at 130° C. for 2 hours. The reaction was cooled to RT, MeOH (10 mL) was added and the mixture stirred at RT for 1 hour, then concentrated at reduced pressure. The residue was purified by Biotage Isolera™ chromatography (silica gel, eluting with DCM/MeOH, 100:0 to 80:20) to afford 1.33 g (68% yield) of the title compound as a yellow solid.
¹H NMR (500 MHz, DMSO-d6) δ [ppm] 8.47-8.43 (m, 2H), 6.96 (d, J=8.3 Hz, 1H), 6.76-6.69 (m, 2H), 3.77 (s, 3H), 3.61 (s, 3H).
LCMS (Analytical Method A): Rt=1.03 mins; MS (ESIpos) m/z=346.0 (M+H)⁺.
In analogy to the procedure described for Intermediate 64A, the following intermediates were prepared using the corresponding nitrile as starting material.

| Int. | Structure | Name | Analytical Data |
|---|---|---|---|
| 65A | 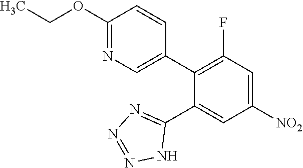 [SMILES: CCOc1ccc(-c2c(F)cc([N+](=O)[O-])cc2-c2nnn[nH]2)cn1] | 2-ethoxy-5-[2-fluoro-4- nitro-6-(1H-tetrazol-5- yl)phenyl]pyridine | ¹H NMR (500 MHz, DMSO-d6) δ [ppm] 8.60-8.49 (m, 1H), 8.39 (dd, J = 9.1, 2.3 Hz, 1H), 8.00 (d, J = 2.3 Hz, 1H), 7.54 (dd, J = 8.6, 2.4 Hz, 1H), 6.82 (d, J = 8.6 Hz, 1H), 4.32 (q, J = 7.0 Hz, 2H), 1.33 (t, J = 7.0 Hz, 3H). LCMS (Analytical Method A): Rt = 1.11 mins; MS (ESIpos) m/z = 330.95 (M + H)⁺. |
| 66A | 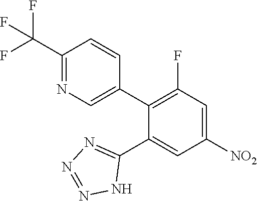 [SMILES: O=[N+]([O-])c1cc(F)c(-c2ccc(C(F)(F)F)nc2)c(-c2nnn[nH]2)c1] | 5-[2-fluoro-4-nitro-6-(1H- tetrazol-5-yl)phenyl]-2- (trifluoromethyl)pyridine | ¹H NMR (500 MHz, DMSO-d6) δ [ppm] 8.69 (s, 2H), 8.49 (d, J = 8.1 Hz, 1H), 8.08 (dd, J = 8.1, 1.7 Hz, 1H), 8.01 (d, J = 8.1 Hz, 1H). LCMS (Analytical Method A): Rt = 1.11 mins; MS (ESIpos) m/z = 354.95 (M + H)⁺. |

### Intermediate 67A: 2-Fluoro-3′,4′-dimethoxy-6-(1H-tetrazol-5-yl)biphenyl-4-amine

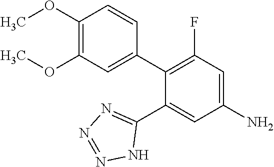 [SMILES: COc1ccc(-c2c(F)cc(N)cc2-c2nnn[nH]2)cc1OC]

To a de-gassed suspension of 5-(6-fluoro-3′,4′-dimethoxy-4-nitrobiphenyl-2-yl)-1H-tetrazole (1.3 g, 3.7 mmol) in EtOH (100 mL) was added Pd/C (5%, 200 mg, 0.094 mmol). The mixture was stirred at room temperature under an atmosphere of hydrogen for a total of 16 hours. The catalyst was removed by filtration through celite and washed with EtOH (50 mL). The filtrate was concentrated in vacuo to afford 1.15 g (97% yield) of the title compound as yellow solid.
¹H NMR (500 MHz, DMSO-d6) δ [ppm] 6.83 (d, J=8.2 Hz, 1H), 6.65-6.58 (m, 2H), 6.55-6.48 (m, 2H), 5.79 (s, 2H), 3.72 (s, 3H), 3.57 (s, 3H).
LCMS (Analytical Method A): Rt=0.92 mins; MS (ESIpos) m/z=316.0 (M+H)⁺.
In analogy to the procedure described for Intermediate 67A, the following intermediates were prepared using the corresponding nitro compound as starting material.

| Int. | Structure | Name | Analytical Data |
|---|---|---|---|
| 68A | 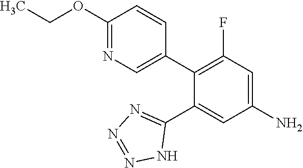 [SMILES: CCOc1ccc(-c2c(F)cc(N)cc2-c2nnn[nH]2)cn1] | 4-(6-ethoxypyridin-3- yl)-3-fluoro-5-(1H- tetrazol-5-yl)aniline | ¹H NMR (500 MHz, DMSO-d6) δ [ppm] 7.80-7.73 (m, 1H), 7.30 (dd, J = 8.5, 2.2 Hz, 1H), 6.72-6.67 (m, 2H), 6.63 (dd, J = 12.3, 2.1 Hz, 1H), 5.91 (s, 2H), 4.26 (q, J = 7.0 Hz, 2H), 1.29 (t, J = 7.0 Hz, 3H). LCMS (Analytical Method A): Rt = 0.99 mins; MS (ESIpos) m/z = 300.95 (M + H)⁺. |
| 69A | 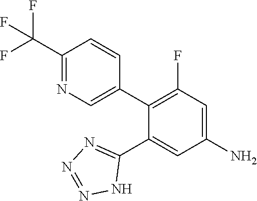 [SMILES: Nc1cc(F)c(-c2ccc(C(F)(F)F)nc2)c(-c2nnn[nH]2)c1] | 3-fluoro-5-(1H-tetrazol- 5-yl)-4-[6- (trifluoromethyl)pyridin- 3-yl]aniline | ¹H NMR (500 MHz, DMS0-d6) δ [ppm] 8.43 (s, 1H), 7.83 (d, J = 8.1 Hz, 1H), 7.73 (d, J = 8.0 Hz, 1H), 6.85 (s, 1H), 6.68 (dd, J = 12.6, 2.1 Hz, 1H), 6.10 (s, 2H). LCMS (Analytical Method F): Rt = 2.44 mins; MS (ESIpos) m/z = 325.1 (M + H)⁺. |

### Intermediate 70A: 2-(6-ethoxypyridin-3-yl)-4-hydroxy-5-nitrobenzonitrile

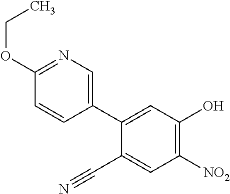 [SMILES: CCOc1ccc(-c2cc(O)c([N+](=O)[O-])cc2C#N)cn1]

Water (37 mL) and 1,2-dimethoxyethane (74 mL) were degassed with a stream of argon for 15 mins. Then 2-bromo-4-fluoro-5-nitrobenzonitrile (5.00 g, 20.4 mmol), 2-ethoxy-5-pyridine boronic acid (3.41 g, 20.4 mmol), potassium carbonate (9.31 g, 67.3 mmol) and dichlorobis(triphenylphosphine) palladium(II) (172 mg, 0.245 mmol) were added, and the reaction heated at 90° C. for 5 h. The mixture was cooled and put into water. Ethyl acetate was added and the layers separated. The aqueous layer was extracted 3× with EE. The combined organic layers were washed with brine, dried with sodium sulfate and concentrated under reduced pressure. The residue was purified by Biotage Isolera™ chromatography (silica gel, eluting with heptane/ethyl acetate/MeOH, 100:0:0 to 0:100:0 to 0:70:30) to yield 1.63 g (25% yield) of the title compound (where the F atom is replaced by hydroxy).
¹H NMR (400 MHz, DMSO-d6) δ [ppm] 1.35 (t, 3H), 4.37 (q, 2H), 6.73 (s, 1H), 6.92 (dd, 1H), 7.87 (dd, 1H), 8.29 (s, 1H), 8.31 (d, 1H).
LCMS (method 1): Rₜ=1.06; MS (ESIpos) m/z=286 (M+H)⁺.

In analogy to the procedure described for Intermediate 70A, the following intermediate was prepared using the corresponding pyridine boronic acid or pyridine boronic pinacol ester as starting material.

| Int. | Structure | Name | Analytical Data |
|---|---|---|---|
| 71A | 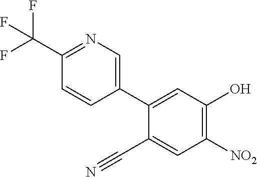 [SMILES: N#Cc1cc([N+](=O)[O-])c(O)cc1-c1ccc(C(F)(F)F)nc1] | 4-hydroxy-5-nitro-2-[6- (trifluoromethyl)pyridin- 3-yl]benzonitrile | ¹H NMR (400 MHz, DMSO-d6) δ [ppm] 6.72 (s, 1H), 8.05 (d, 1H), 8.26 (dd, 1H), 8.29 (s, 1H), 8.91 (d, 1H); LCMS (method 1): Rₜ = 1.01; MS (ESIpos) m/z = 310 (M + H)⁺. |

In analogy to the procedure described for Intermediate 54A, the following intermediates were prepared using the corresponding nitrile as starting material.

| Int. | Structure | Name | Analytical Data |
|---|---|---|---|
| 72A | 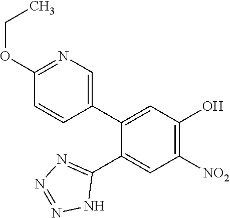 [SMILES: CCOc1ccc(-c2cc(O)c([N+](=O)[O-])cc2-c2nnn[nH]2)cn1] | 5-(6-ethoxypyridin-3- yl)-2-nitro-4-(1H- tetrazol-5-yl)phenol | ¹H NMR (400 MHz, DMSO-d6) δ [ppm] 1.32 (t, 3H), 4.31 (q, 2H), 6.73 (d, 1H), 7.18 (s, 1H), 7.40 (dd, 1H), 7.98 (d, 1H), 8.28 (d, 1H). LCMS (method 1): Rt = 0.85; MS (ESIpos) m/z = 329 (M + H)⁺. |
| 73A | 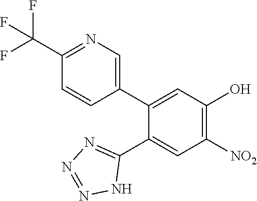 [SMILES: O=[N+]([O-])c1cc(-c2nnn[nH]2)c(-c2ccc(C(F)(F)F)nc2)cc1O] | 2-nitro-4-(1H-tetrazol- 5-yl)-5-[6- (trifluoromethyl)pyridin- 3-yl]phenol | ¹H NMR (400 MHz, DMSO-d6) δ [ppm] 7.17 (s, 1H), 7.90 (m, 2H), 8.40 (s, 1H), 8.57 (s, 1H); LCMS (method 1): Rₜ = 0.85; MS (ESIpos) m/z = 353 (M + H)⁺. |

In analogy to the procedure described for Intermediate 57A, the following intermediates were prepared using the corresponding nitro compound as starting material.

| Int. | Structure | Name | Analytical Data |
|---|---|---|---|
| 74A | 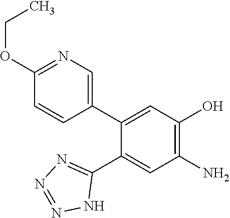 [SMILES: CCOc1ccc(-c2cc(O)c(N)cc2-c2nnn[nH]2)cn1] | 2-amino-5-(6- ethoxypyridin-3-yl)-4- (1H-tetrazol-5-yl)phenol | ¹H NMR (400 MHz, DMSO-d6) δ [ppm] 1.30 (t, 3H), 4.26 (q, 2H), 6.62 (d, 1H), 6.72 (s, 1H), 6.88 (s, 1H), 7.22 (dd, 1H), 7.78 (d, 1H). LCMS (method 1): Rₜ = 0.54; MS (ESIpos) m/z = 299 (M + H)⁺. |
| 75A | 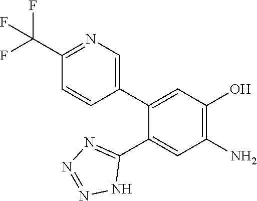 [SMILES: Nc1cc(-c2nnn[nH]2)c(-c2ccc(C(F)(F)F)nc2)cc1O] | 2-amino-4-(1H-tetrazol- 5-yl)-5-[6- (trifluoromethyl)pyridin- 3-yl]phenol | ¹H NMR (400 MHz, DMSO-d6) δ [ppm] 6.78 (s, 1H), 6.99 (s, 1H), 7.62 (dd, 1H), 7.74 (d, 1H), 8.35 (d, 1H). LCMS (method 1): Rₜ = 0.62; MS (ESIpos) m/z = 323 (M + H)⁺. |

### Intermediate 76A: 5-Nitro-2-[4-(trifluoromethyl)-1H-pyrazol-1-yl]benzonitrile

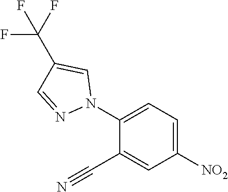 [SMILES: N#Cc1cc([N+](=O)[O-])ccc1-n1cc(C(F)(F)F)cn1]

To a solution of 2-chloro-5-nitrobenzonitrile (1.278 g, 7.0 mmol) and 4-(trifluoromethyl)-1H-pyrazole (1.0 g, 7.35 mmol) in acetonitrile (10 mL) was added potassium carbonate (2.90 g, 21 mmol) at RT. The mixture was heated to 60° C. and stirred at this temperature for 1 hour. The reaction mixture was diluted with EtOAc (50 mL), and the solids were removed by filtration. The filtrate was concentrated in vacuo and the residue was purified by Biotage Isolera™ chromatography (silica gel, eluting with heptanes—EtOAc, 1:0 to 0:1). The product containing fractions were combined and concentrated in vacuo to afford 1.77 g (89% yield) of the title compound as a yellow solid.
¹H NMR (500 MHz, DMSO-d6) δ [ppm] 9.28 (s, 1H), 8.94 (d, J=2.6 Hz, 1H), 8.68 (dd, J=9.0, 2.6 Hz, 1H), 8.47 (s, 1H), 8.19 (d, J=9.0 Hz, 1H).
LCMS (Analytical Method A): Rt=1.16 mins; MS (ESIpos) m/z=283.0 (M+H)⁺.
In analogy to the procedure described for Intermediate 76A, the following intermediates were prepared:

| Int. | Structure | Name | Analytical Data |
|---|---|---|---|
| 77A | 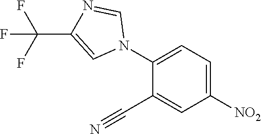 [SMILES: N#Cc1cc([N+](=O)[O-])ccc1-n1cnc(C(F)(F)F)c1] | 5-Nitro-2-[4- (trifluoromethyl)- 1H-imidazol- 1-yl]benzonitrile | ¹H NMR (500 MHz, DMSO-d6) δ [ppm] 9.04 (d, J = 2.6 Hz, 1H), 8.70 (dd, J = 8.9, 2.6 Hz, 1H), 8.52-8.49 (m, 1H), 8.47 (s, 1H), 8.10 (d, J = 8.9 Hz, 1H). LCMS (Analytical Method A): Rt = 1.06 mins; MS (ESIpos) m/z = 283 (M + H)⁺. |
| 78A | 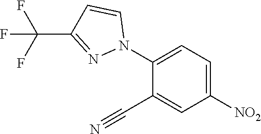 [SMILES: N#Cc1cc([N+](=O)[O-])ccc1-n1ccc(C(F)(F)F)n1] | 5-Nitro-2-[3- (trifluoromethyl)- 1H-pyrazol-1- yl]benzonitrile | ¹H NMR (500 MHz, DMSO-d6) δ [ppm] 8.96 (d, J = 2.6 Hz, 1H), 8.84- 8.77 (m, 1H), 8.68 (dd, J = 9.0, 2.6 Hz, 1H), 8.18 (d, J = 9.0 Hz, 1H), 7.23 (d, J = 2.7 Hz, 1H). LCMS (Analytical Method A): Rt = 1.16 mins. |
| 79A | 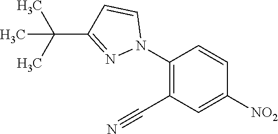 [SMILES: CC(C)(C)c1ccn(-c2ccc([N+](=O)[O-])cc2C#N)n1] | 2-(3-tert-butyl-1H- pyrazol-1-yl)-5- nitrobenzonitrile | ¹H NMR (500 MHz, DMSO-d6) δ [ppm] 8.77 (d, J = 2.6 Hz, 1H), 8.57 (dd, J = 9.2, 2.6 Hz, 1H), 8.54 (d, J = 2.7 Hz, 1H), 8.10 (d, J = 9.2 Hz, 1H), 6.68 (d, J = 2.7 Hz, 1H), 1.33 (s, 9H). LCMS (Analytical Method A): Rt = 1.28 mins; MS (ESIpos) m/z = 271 (M + H)⁺. |
| 80A | 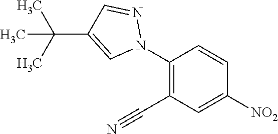 [SMILES: CC(C)(C)c1cnn(-c2ccc([N+](=O)[O-])cc2C#N)c1] | 2-(4-tert-butyl-1H- pyrazol-1-yl)-5- nitrobenzonitrile | ¹H NMR (500 MHz, DMSO-d6) δ [ppm] 8.80 (d, J = 2.6 Hz, 1H), 8.58 (dd, J = 9.2, 2.6 Hz, 1H), 8.46 (d, J = 0.6 Hz, 1H), 8.12 (d, J = 9.2 Hz, 1H), 7.99 (d, J = 0.5 Hz, 1H), 1.30 (s, 9H). LCMS (Analytical Method A): Rt = 1.26 mins; MS (ESIPos) m/z = 271 [M + H]⁺. |
| 81A | 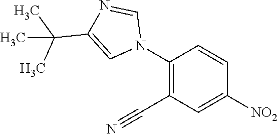 [SMILES: CC(C)(C)c1cn(-c2ccc([N+](=O)[O-])cc2C#N)cn1] | 2-(4-tert-butyl-1H- imidazol-1-yl)-5- nitro-benzonitrile | ¹H NMR (250 MHz, DMSO-d6) δ [ppm] 8.93 (d, J = 2.6 Hz, 1H), 8.61 (dd, J = 9.0, 2.7 Hz, 1H), 8.18 (d, J = 1.4 Hz, 1H), 7.97 (d, J = 9.0 Hz, 1H), 7.48 (d, J = 1.4 Hz, 1H), 1.28 (s, 9H). LCMS (Analytical Method A): Rt = 0.96 mins, MS (ESIPos) m/z = 271 [M + H]⁺. |

Alternatively, analogous compounds can be prepared via the following protocol:

### Intermediate 82A: 2-(3-Ethyl-2-oxo-pyrrolidin-1-yl)-5-nitro-benzonitrile

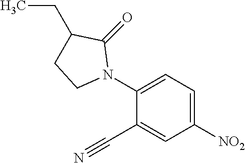 [SMILES: CCC1CCN(c2ccc([N+](=O)[O-])cc2C#N)C1=O]

To a solution of 3-ethylpyrrolidin-2-one (0.89 g, 7.865 mmol) in DMF (25 mL) was added sodium hydride (60%, 346 mg, 8.651 mmol) at 0° C. (ice-bath). The mixture was stirred for 30 minutes at 0° C., then the ice-bath was removed and the mixture stirred for another 30 minutes at RT. A solution of 2-fluoro-5-nitrobenzonitrile (1.306 g, 7.865 mmol) in DMF (5 mL) was added dropwise via syringe and the resulting dark red solution was stirred for 1 hour at RT. The reaction mixture was poured into a mixture of 2M HCl (50 mL) and crushed ice (˜50 g) and the organics were extracted with EtOAc (3×50 mL). The combined organic extracts were washed with water (80 mL) and brine (50 mL), dried (Na₂SO₄) and concentrated at reduced pressure. Purification by Biotage Isolera™ chromatography (silica gel, eluting with heptanes—EtOAc, 1:0 to 1:1) afforded the title compound (racemic mixture; 1.18 g, 57% yield) as pale yellow solid.
¹H NMR (500 MHz, DMSO-d6) δ [ppm] 8.74 (d, J=2.7 Hz, 1H), 8.54 (dd, J=9.0, 2.7 Hz, 1H), 7.79 (d, J=9.0 Hz, 1H), 4.05-3.97 (m, 1H), 3.87-3.80 (m, 1H), 2.66-2.57 (m, 1H), 2.37-2.28 (m, 1H), 1.91-1.73 (m, 2H), 1.58-1.46 (m, 1H), 0.97 (t, J=7.5 Hz, 3H);
LCMS (Analytical Method A): Rt=1.06 mins, MS (ESIpos): m/z=259.95 (M+H)⁺.
In analogy to the procedure described for Intermediate 76A, the following intermediate was prepared:

| Int. | Structure | Name | Analytical Data |
|---|---|---|---|
| 83A | 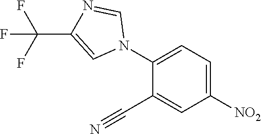 [SMILES: N#Cc1cc([N+](=O)[O-])ccc1-n1cnc(C(F)(F)F)c1] | 5-Nitro-2-[4- (trifluoromethyl)- 1H-imidazol- 1-yl]benzonitrile | ¹H NMR (500 MHz, DMSO-d6) δ [ppm] 9.04 (d, J = 2.6 Hz, 1H), 8.70 (dd, J = 8.9, 2.6 Hz, 1H), 8.52-8.49 (m, 1H), 8.47 (s, 1H), 8.10 (d, J = 8.9 Hz, 1H). LCMS (Analytical Method A): Rt = 1.06 mins; MS (ESIpos) m/z = 283 (M + H)⁺. |

### Intermediate 84A: 5-{5-Nitro-2-[4-(trifluoromethyl)-1H-pyrazol-1-yl]phenyl}-1H-tetrazole

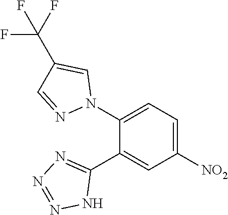 [SMILES: O=[N+]([O-])c1ccc(-n2cc(C(F)(F)F)cn2)c(-c2nnn[nH]2)c1]

A pressure tube (ACE) was loaded with 5-nitro-2-[4-(trifluoromethyl)-1H-pyrazol-1-yl]benzonitrile (1.77 g, 6.21 mmol), p-xylene (8 mL), di-n-butyltin oxide (1.546 g, 6.21 mmol) and azidotrimethylsilane (1.65 mL, 12.42 mmol). The pressure tube was sealed and heated with stirring at 130° C. for 1 hour. The reaction was cooled to RT, MeOH (10 mL) was added and the mixture stirred at RT for 1 hour, then concentrated at reduced pressure. The residue was purified by Biotage Isolera™ chromatography (silica gel, eluting with DCM-MeOH, 1:0 to 4:1) The product containing fractions were combined and concentrated at reduced pressure to give 1.7 g (67% yield) of the title compound as a yellow oil.
¹H NMR (500 MHz, DMSO-d6) δ [ppm] 8.84 (s, 1H), 8.72 (d, J=2.6 Hz, 1H), 8.54 (dd, J=8.8, 2.6 Hz, 1H), 8.11 (s, 1H), 8.04 (d, J=8.8 Hz, 1H).
LCMS (Analytical Method A): Rt=1.03 mins; MS (ESIpos) m/z=326.05 (M+H)⁺.
In analogy to the procedure described for Intermediate 84A, the following intermediates were prepared:

| Int. | Structure | Name | Analytical Data |
|---|---|---|---|
| 85A | 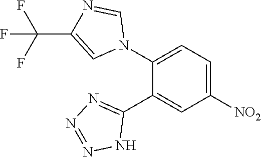 [SMILES: O=[N+]([O-])c1ccc(-n2cnc(C(F)(F)F)c2)c(-c2nnn[nH]2)c1] | 5-{5-Nitro-2-[4- (trifluoromethyl)- 1H-imidazol-1- yl]phenyl}- 1H-tetrazole | ¹H NMR (500 MHz, DMSO-d6) δ [ppm] 8.86 (d, J = 2.6 Hz, 1H), 8.57 (dd, J = 8.7, 2.6 Hz, 1H), 8.10-8.06 (m, 1H), 8.04 (s, 1H), 8.02 (d, J = 8.7 Hz, 1H). LCMS (Analytical Method A): Rt = 0.96 mins; MS (ESIpos) m/z = 326.0 (M + H)⁺. |
| 86A | 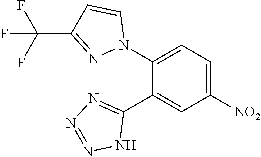 [SMILES: O=[N+]([O-])c1ccc(-n2ccc(C(F)(F)F)n2)c(-c2nnn[nH]2)c1] | 5-{5-Nitro-2-[3- (trifluoromethyl)- 1H-pyrazol-1- yl]phenyl}-1H- tetrazole | ¹H NMR (500 MHz, DMSO-d6) δ [ppm] 8.74 (d, J = 2.6 Hz, 1H), 8.60 (dd, J = 8.8, 2.6 Hz, 1H), 8.41-8.35 (m, 1H), 8.10 (d, J = 8.8 Hz, 1H), 7.01 (d, J = 2.6 Hz, 1H). LCMS (Analytical Method A): Rt = 1.04 mins; MS (ESIpos) m/z = 326.0 (M + H)⁺. |

In analogy to the procedure described for Intermediate 57A, the following intermediates were prepared:

| Int. | Structure | Name | Analytical Data |
|---|---|---|---|
| 87A | 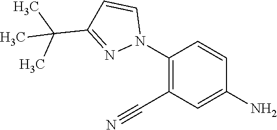 [SMILES: CC(C)(C)c1ccn(-c2ccc(N)cc2C#N)n1] | 5-amino-2-(3- tert-butyl-1H- pyrazol-1- yl)benzonitrile | ¹H NMR (500 MHz, DMSO-d6) δ [ppm] 7.95 (d, J = 2.4 Hz, 1H), 7.35 (d, J = 8.7 Hz, 1H), 6.95 (d, J = 2.5 Hz, 1H), 6.92 (dd, J = 8.7, 2.6 Hz, 1H), 6.37 (d, J = 2.4 Hz, 1H), 5.74 (s, 2H), 1.29 (s, 9H). LCMS (Analytical Method A): Rt = 1.13 mins; MS (ESIpos) m/z = 241 (M + H)⁺. |
| 88A | 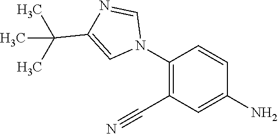 [SMILES: CC(C)(C)c1cn(-c2ccc(N)cc2C#N)cn1] | 5-amino-2-(4- tert-butyl-1H- imidazol-1- yl)benzonitrile | ¹H NMR (500 MHz, DMSO-d6) δ [ppm] 7.73 (d, J = 1.3 Hz, 1H), 7.26 (d, J = 8.7 Hz, 1H), 7.07 (d, J = 1.4 Hz, 1H), 6.96 (d, J = 2.6 Hz, 1H), 6.92 (dd, J = 8.7, 2.6 Hz, 1H), 5.83 (s, 2H), 1.24 (s, 9H). LCMS (Analytical Method A): Rt = 0.75 mins; MS (ESIpos) m/z = 241 (M + H)⁺. |
| 89A | 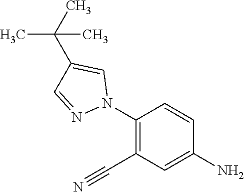 [SMILES: CC(C)(C)c1cnn(-c2ccc(N)cc2C#N)c1] | 5-amino-2-(4- tert-butyl-1H- pyrazol-1- yl)benzonitrile | ¹H NMR (500 MHz, DMSO-d6) δ [ppm] 7.92-7.86 (m, 1H), 7.67-7.60 (m, 1H), 7.35 (d, J = 8.7 Hz, 1H), 6.99-6.88 (m, 2H), 1.26 (s, 9H). LCMS (Analytical Method A): Rt = 1.12 mins; MS (ESIPos) m/z = 241 [M + H]⁺. |
| 90A | 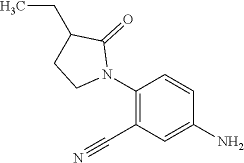 [SMILES: CCC1CCN(c2ccc(N)cc2C#N)C1=O] | 5-amino-2-(4- ethyl-1H- pyrazol-1- yl)benzonitrile, as a racemic mixture | 1H NMR (500 MHz, Chloroform-d) δ 7.12-7.07 (m, 1H), 6.88-6.81 (m, 2H), 3.95 (s, 2H), 3.80-3.67 (m, 2H), 2.62-2.50 (m, 1H), 2.41-2.29 (m, 1H), 2.02-1.85 (m, 2H), 1.64-1.48 (m, 1H), 1.03 (t, J = 7.5 Hz, 3H). LCMS (Analytical Method A): Rt = 0.91 mins, MS (ESIpos): m/z = 229.95 (M + H)⁺. |

### Intermediate 91A: 3-(1H-Tetrazol-5-yl)-4-[4-(trifluoromethyl)-1H-pyrazol-1-yl]aniline

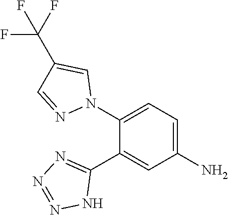 [SMILES: Nc1ccc(-n2cc(C(F)(F)F)cn2)c(-c2nnn[nH]2)c1]

To a degassed solution of 5-{5-nitro-2-[4-(trifluoromethyl)-1H-pyrazol-1-yl]phenyl}-1H-tetrazole (1.7 g, 4.18 mmol) in EtOH (30 mL) was added Pd/C (10%, 100 mg, 0.094 mmol). The mixture was stirred at room temperature under an atmosphere of hydrogen for 18 hours. The catalyst was removed by filtration through Celite® and washed with EtOH (50 mL). The filtrate was concentrated in vacuo. The residue was purified by preparative HPLC (Method B) to give 1.09 g (87% yield) of the title compound as white solid.
¹H NMR (500 MHz, DMSO-d6) δ [ppm] 8.52 (s, 1H), 7.86 (s, 1H), 7.32 (d, J=8.6 Hz, 1H), 6.94 (s, 1H), 6.83 (dd, J=8.6, 2.6 Hz, 1H), 5.86 (s, 2H).
LCMS (Analytical Method F): Rt=2.16 mins; MS (ESIpos) m/z=296 (M+H)⁺.
In analogy to the procedure described for Intermediate 91A, the following intermediates were prepared:

| Int. | Structure | Name | Analytical Data |
|---|---|---|---|
| 92A | 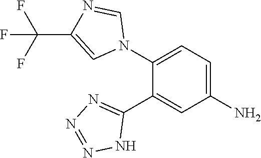 [SMILES: Nc1ccc(-n2cnc(C(F)(F)F)c2)c(-c2nnn[nH]2)c1] | 3-(1H-Tetrazol- 5-yl)-4-[4- (trifluoromethyl)- 1H-imidazol-1- yl]aniline | ¹H NMR (500 MHz, DMSO-d6) δ [ppm] 7.75 (s, 1H), 7.74 (s, 1H), 7.27 (d, J = 8.6 Hz, 1H), 7.04 (s, 1H), 6.82 (dd, J = 8.6, 2.6 Hz, 1H), 5.88 (s, 2H). LCMS (Analytical Method F): Rt = 1.86 mins; MS (ESIpos) m/z = 296.1 (M + H)⁺. |
| 93A | 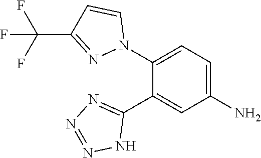 [SMILES: Nc1ccc(-n2ccc(C(F)(F)F)n2)c(-c2nnn[nH]2)c1] | 3-(1H-Tetrazol- 5-yl)-4-[3- (trifluoromethyl)- 1H-pyrazol-1- yl]aniline | ¹H NMR (500 MHz, DMSO-d6) δ [ppm] 8.02-7.94 (m, 1H), 7.34 (d, J = 8.6 Hz, 1H), 6.94 (s, 1H), 6.84 (dd, J = 8.6, 2.6 Hz, 1H), 6.78 (d, J = 2.4 Hz, 1H), 5.87 (s, 2H). LCMS (Analytical Method F): Rt = 2.14 mins; MS (ESIpos) m/z = 296.1 (M + H)⁺. |
| 94A | 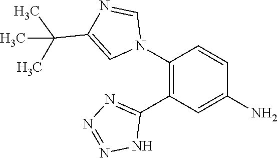 [SMILES: CC(C)(C)c1cn(-c2ccc(N)cc2-c2nnn[nH]2)cn1] | 4-(4-tert-butyl-1H- imidazol-1-yl)-3- (1H-tetrazol-5- yl)aniline | ¹H NMR (500 MHz, DMSO-d6) δ 8.32-8.18 (m, 1H), 7.18 (d, J = 8.5 Hz, 1H), 7.14-7.04 (m, 1H), 6.97- 6.89 (m, 1H), 6.70 (dd, J = 8.5, 2.6 Hz, 1H), 1.21 (s, 9H). LCMS (Analytical Method F): Rt = 1.29 mins; MS (ESIpos) m/z = 284 (M + H)⁺. |
| 95A | 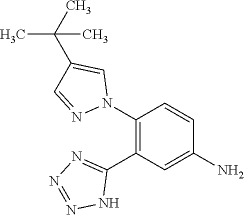 [SMILES: CC(C)(C)c1cnn(-c2ccc(N)cc2-c2nnn[nH]2)c1] | 4-(4-tert- butylpyrazol-1- yl)-3-(1H- tetrazol-5- yl)aniline | ¹H NMR (500 MHz, DMSO-d6) δ [ppm] 7.45 (s, 1H), 7.34 (s, 1H), 7.28 (d, J = 8.6 Hz, 1H), 6.90-6.68 (m, 2H), 1.18 (s, 9H). LCMS (Analytical Method F): Rt = 2.43 mins; MS (ESIPos) m/z = 284 [M + H]⁺. |

### Intermediate 96A −4-(3-tert-butyl-1H-pyrazol-1-yl)-3-(1H-tetrazol-5-yl)aniline

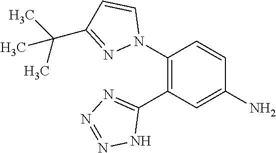 [SMILES: CC(C)(C)c1ccn(-c2ccc(N)cc2-c2nnn[nH]2)n1]

A pressure tube (ACE) was loaded with 5-amino-2-(3-tert-butyl-1H-pyrazol-1-yl)benzonitrile (1.05 g, 4.33 mmol), p-xylene (6 mL), di-n-butyltin oxide (1.077 g, 4.326 mmol) and azidotrimethylsilane (0.861 mL, 6.49 mmol). The pressure tube was sealed and heated with stirring at 130° C. for 2 hours. The reaction was cooled to RT, MeOH (10 mL) was added and the mixture stirred at RT for 1 hour, then concentrated at reduced pressure. The crude material was purified by preparative HPLC (Method B) to afford 0.91 g (74% yield) of the title compound as white solid.
¹H NMR (500 MHz, DMSO-d6) δ [ppm] 7.68 (m, 1H), 7.29 (m, 1H), 6.82 (m, 1H), 6.78 (m, 1H), 6.18 (m, 1H), 5.65 (s, 2H), 1.04 (s, 9H)
LCMS (Analytical Method F): Rt=2.36 mins; MS (ESIpos) m/z=284 (M+H)⁺.
In analogy to the procedure described for Intermediate 96A, the following intermediate was prepared:

| Int. | Structure | Name | Analytical Data |
|---|---|---|---|
| 97A | 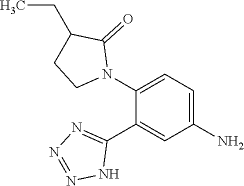 [SMILES: CCC1CCN(c2ccc(N)cc2-c2nnn[nH]2)C1=O] | 1-[4-amino-2- (1H-tetrazol-5- yl)phenyl]-3- ethylpyrrolidin- 2-one, as a racemic mexture | ¹H NMR (500 MHz, DMSO-d6) δ [ppm] 7.08 (d, J = 8.5 Hz, 1H), 6.88 (s, 1H), 6.76 (dd, J = 8.5, 2.6 Hz, 1H), 5.54 (s, 2H), 3.73-3.62 (m, 1H), 3.61-3.52 (m, 1H), 2.25-2.13 (m, 2H), 1.82- 1.67 (m, 1H), 1.62-147 (m, 1H), 1.31- 1.14 (m, 1H), 0.82 (t, J = 7.4 Hz, 3H). LCMS (Analytical Method F): Rt = 1.61 mins; MS (ESIpos) m/z = 273.1 (M + H)⁺. |

### Intermediate 98A: Ethyl-1-methyl-2-phenylcyclopropane-1-carboxylate, as a mixture of trans enantiomers

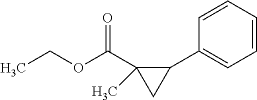

Triethyl 2-phosphonopropionate (1.0 mL, 4.7 mmol) was dissolved in 1,2-dimethoxyethane (20 mL) and n-Butyllithium (1.9 mL of a 2.5M soln in heptane, 4.7 mmol) was added dropwise at 0° C. After 5 minutes, racemic 2-phenyloxirane (0.27 mL, 2.3 mmol) was added dropwise and the resulting solution was heated at 100° C. overnight. The following day the mixture was cooled to room temperature and quenched with saturated aqueous ammonium chloride (10 mL). The mixture was diluted with ethyl acetate (50 mL) and washed with brine (2×30 mL), dried (sodium sulfate), filtered and concentrated at reduced pressure. The residue was purified by Biotage Isolera™ chromatography (silica gel, eluting with heptanes—EtOAc, 99:1 to 7:3) giving the title compound (386 mg, 41% yield) as a colourless oil.

### ¹H NMR (500 MHz, Chloroform-d) δ 7.40-7.16 (m, 5H), 4.25-4.16 (m, 2H), 2.87-2.75 (m, 1H), 1.76-1.67 (m, 1H), 1.31 (t, J=7.1 Hz, 3H), 1.21-1.15 (m, 1H), 1.01 (s, 3H)

LCMS (Analytical Method A): Rt=1.33 mins; MS (ESIpos) m/z=205 (M+H)⁺.
Intermediate 99A: 1-Methyl-2-phenylcyclopropane-1-carboxylic acid, as a mixture of trans enantiomers

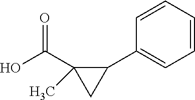

Ethyl 1-methyl-2-phenylcyclopropane-1-carboxylate, as a mixture of trans enantiomers (386 mg, 1.9 mmol) was dissolved in THF (5 mL) and lithium hydroxide monohydrate (159 mg, 3.8 mmol) was dissolved in water (5 mL) and added giving a cloudy white suspension. The reaction was stirred for 2 days at room temperature. Further lithium hydroxide monohydrate (159 mg, 3.8 mmol) was then added and the reaction was allowed to stir overnight. The mixture was acidified with 2M aq. HCl (6 mL) and extracted with EtOAc (50 mL). The organics were washed with brine (20 mL), dried (sodium sulfate), filtered and concentrated at reduced pressure. The residue was purified by Biotage Isolera™ chromatography (silica gel, eluting with heptanes—EtOAc, 98:2 to 1:1) giving the title compound (176 mg, 53% yield) as a white solid. The product was isolated as a racemic single disastereoisomer.

### ¹H NMR (250 MHz, Chloroform-d) δ 7.43-7.13 (m, 5H), 3.04-2.83 (m, 1H), 1.88-1.72 (m, 1H), 1.37-1.23 (m, 1H), 1.02 (s, 3H)

LCMS (Analytical Method A): Rt=1.10 mins; The product did not ionise by LCMS.

### Example 1

### N[3′,4′-dimethoxy-2-(1H-tetrazol-5-yl)biphenyl-4-yl]-2-phenylcyclopropanecarboxamide, as a Mixture of Trans Enantiomers

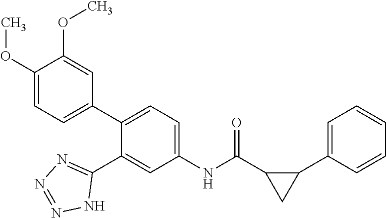 [SMILES: COc1ccc(-c2ccc(NC(=O)C3CC3c3ccccc3)cc2-c2nnn[nH]2)cc1OC]

To a solution of 2-phenylcyclopropanecarboxylic acid, as a mixture of trans enantiomers (110 mg, 0.68 mmol) in N,N-dimethylformamide (3 mL) was added HATU (281 mg, 0.74 mmol) followed by DIPEA (153 μL, 0.88 mmol). To this solution was added 3′,4′-dimethoxy-2-(1H-tetrazol-5-yl)biphenyl-4-amine (Intermediate 17A) (200 mg, 0.67 mmol) and the reaction was stirred under nitrogen at room temperature for 6 h. The reaction mixture was partitioned between EtOAc (20 mL) and 1M HCl (20 mL), the layers separated and organics washed with brine (20 mL), dried (sodium sulfate), filtered and concentrated at reduced pressure. The crude residue was triturated in boiling MeCN (10 mL), cooled to RT, filtered then washed with MeCN (2 mL) to give the title compound (205 mg, 69% yield) as an off-white crystalline solid.
¹H NMR (500 MHz, DMSO-d6) δ [ppm] 1.36-1.44 (m, 1H), 1.49-1.56 (m, 1H), 2.07-2.13 (m, 1H), 2.40 (m, 1H), 3.58 (s, 3H), 3.73 (s, 3H), 6.52-6.65 (m, 2H), 6.87 (d, J=8.2 Hz, 1H), 7.21 (m, 3H), 7.30 (m, 2H), 7.53 (m, 1H), 7.80 (dd, J=8.5, 2.2 Hz, 1H), 7.93 (d, J=1.9 Hz, 1H), 10.55 (s, 1H).
LCMS (Analytical Method F): Rt=3.21 mins; MS (ESIPos): m/z=442 (M+H)⁺.
In analogy to Example 1, the following examples were prepared using the corresponding amine and carboxylic acid as starting materials:

| Ex. | Structure | Name | Analytical Data |
|---|---|---|---|
| 2 | 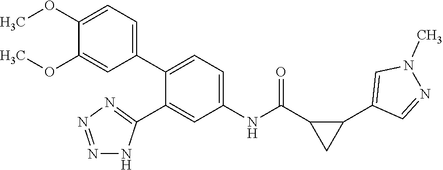 | N-[3′,4′-dimethoxy-2- (1H-tetrazol-5- yl)biphenyl-4-yl]-2- (1-methyl-1H-pyrazol-4- yl)cyclopropanecarbox- amide, as a mixture of trans enantiomers | ¹H NMR (250 MHz, DMSO-d6) δ [ppm] 1.12-1.29 (m, 1H), 1.29-1.48 (m, 1H), 1.81-2.00 (m, 1H), 2.09- 2.28 (m, 1H), 3.58 (s, 3H), 3.73 (s, 3H), 3.77 (s, 3H), 6.49-6.69 (m, 2H), 6.87 (d, J = 8.4 Hz, 1H), 7.29 (s, 1H), 7.41-7.64 (m, 2H), 7.80 (dd, J = 8.5, 2.3 Hz, 1H), 7.91 (d, J = 2.1 Hz, 1H), 10.49 (s, 1H). LCMS (Analytical Method F): Rt = |
| | | | 2.33 mins; MS (ESIPos) m/z = |
| | | | 446.1 (M + H)⁺. |
| 3 | 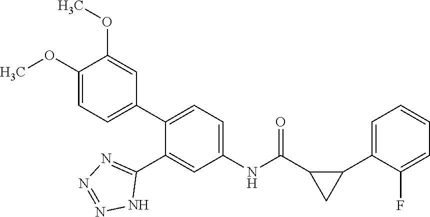 [SMILES: COc1ccc(-c2ccc(NC(=O)C3CC3c3ccccc3F)cc2-c2nnn[nH]2)cc1OC] | N-[3′,4′-dimethoxy- 2-(1H-tetrazol-5- yl)biphenyl-4-yl]-2-(2- fluorophenyl)cyclopro- panecarboxamide, as a mixture of trans enantiomers | ¹H NMR (500 MHz, DMSO-d6) δ [ppm] 10.58 (s, 1H), 7.94 (s, 1H), 7.81 (m, 1H), 7.54 (d, J = 8.5 Hz, 1H), 7.27 (m, 1H), 7.22-7.14 (m, 3H), 6.88 (d, J = 8.2 Hz, 1H), 6.62- 6.55 (m, 2H), 3.73 (s, 3H), 3.58 (s, 3H), 2.53-2.52 (m, 1H), 2.14 (m, 1H), 1.50 (m, 2H). LCMS (Analytical Method D): Rt = 4.08 mins; MS (ESIPos): m/z = 460 (M + H)⁺ |
| 4 | 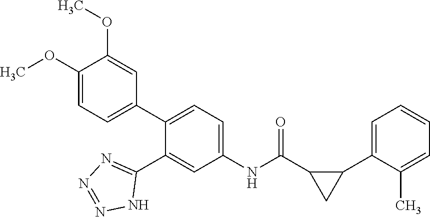 [SMILES: COc1ccc(-c2ccc(NC(=O)C3CC3c3ccccc3C)cc2-c2nnn[nH]2)cc1OC] | N-[3′,4′-dimethoxy-2- (1H-tetrazol-5- yl)biphenyl-4-yl]-2-(2- methylphenyl)cyclopro- panecarboxamide, as a mixture of trans enantiomers | ¹H NMR (500 MHz, DMSO-d6) δ [ppm] 1.37-1.45 (m, 1H), 1.44-1.52 (m, 1H), 1.89-1.95 (m, 1H), 2.32- 2.42 (m, 4H), 3.58 (s, 3H), 3.73 (s, 3H), 6.47-6.65 (m, 2H), 6.88 (d, J = 8.2 Hz, 1H), 7.01-7.25 (m, 4H), 7.54 (d, J = 8.3 Hz, 1H), 7.82 (dd, J = 8.5, 2.3 Hz, 1H), 7.95 (s, 1H), 10.55 (s, 1H). LCMS (Analytical Method F): Rt = 3.38 mins; MS (ESIPos) m/z = 456.2 (M + H)⁺ |
| 5 | 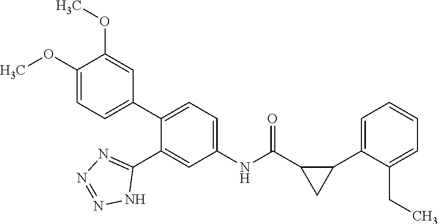 [SMILES: CCc1ccccc1C1CC1C(=O)Nc1ccc(-c2ccc(OC)c(OC)c2)c(-c2nnn[nH]2)c1] | N-[3′,4′-dimethoxy-2- (1H-tetrazol-5- yl)biphenyl-4-yl]-2-(2- ethylphenyl)cyclopro- panecarboxamide, as a mixture of trans enantiomers | ¹H NMR (500 MHz, DMSO-d6) δ [ppm] 10.58 (s, 1H), 7.97 (s, 1H), 7.83 (m, 1H), 7.55 (m, 1H), 7.24- 7.14 (m, 3H), 7.07 (d, J = 6.8 Hz, 1H), 6.89 (d, J = 8.2 Hz, 1H), 6.63- 6.56 (m, 2H), 3.74 (s, 3H), 3.59 (s, 3H), 2.78-2.69 (m, 2H), 2.47-2.43 (m, 1H), 1.99-1.96 (m, 1H), 1.52- 1.42 (m, 2H), 1.18 (t, J = 7.6 Hz, 3H). LCMS (Analytical Method F): Rt = 4.38 mins; MS (ESIPos) m/z = 470 (M + H)⁺. |
| 6 | 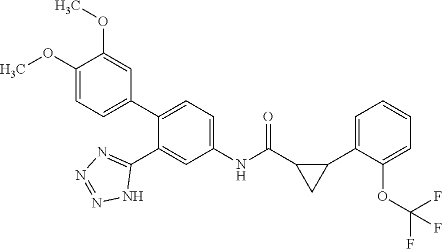 [SMILES: COc1ccc(-c2ccc(NC(=O)C3CC3c3ccccc3OC(F)(F)F)cc2-c2nnn[nH]2)cc1OC] | N-[3′,4′-dimethoxy-2- (1H-tetrazol-5- yl)biphenyl-4-yl]-2-[2- (trifluoromethoxy)phen- yl]cyclopropane- carboxamide, as a mixture of trans enantiomers | ¹H NMR (500 MHz, DMSO-d6) δ [ppm] 1.48-1.64 (m, 2H), 2.00-2.15 (m, 1H), 2.41-2.60 (m, 1H), 3.58 (s, 3H), 3.73 (s, 3H), 6.53-6.64 (m, 2H), 6.88 (d, J = 8.2 Hz, 1H), 7.18- 7.30 (m, 1H), 7.32-7.45 (m, 3H), 7.54 (d, J = 8.5 Hz, 1H), 7.80 (dd, J = 8.5, 2.2 Hz, 1H), 7.89-7.99 (m, 1H), 10.56 (s, 1H). LCMS (Analytical Method F): Rt = 3.54 mins; MS (ESIPos) m/z = 526 (M + H)⁺. |
| 7 | 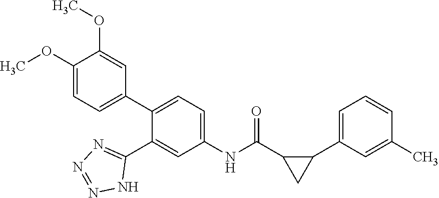 | N-[3′,4′-dimethoxy-2- (1H-tetrazol-5- yl)biphenyl-4-yl]-2-(3- methylphenyl)cyclopro- panecarboxamide, as a mixture of trans enantiomers | ¹H NMR (500 MHz, DMSO-d6) δ [ppm] 1.35-1.42 (m, 1H), 1.46-1.54 (m, 1H), 2.05-2.12 (m, 1H), 2.28 (s, 3H), 2.32-2.41 (m, 1H), 3.58 (s, 3H), 3.73 (s, 3H), 6.52-6.67 (m, 2H), 6.87 (d, J = 8.3 Hz, 1H), 6.95- 7.07 (m, 3H), 7.18 (t, J = 7.5 Hz, 1H), 7.53 (d, J = 8.5 Hz, 1H), 7.79 (dd, J = 8.5, 2.3 Hz, 1H), 7.93 (s, 1H), 10.53 (s, 1H). LCMS (Analytical Method F): Rt = 3.43 mins; MS (ESIPos) m/z = 456 |
| | | | (M + H)⁺ |
| 8 | 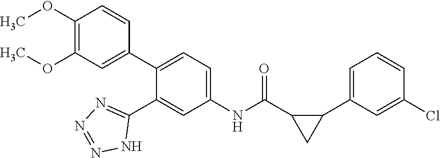 [SMILES: COc1ccc(-c2ccc(NC(=O)C3CC3c3cccc(Cl)c3)cc2-c2nnn[nH]2)cc1OC] | 2-(3-chlorophenyl)- N-[3′,4′-dimethoxy-2- (1H-tetrazol-5- yl)biphenyl-4-yl]cyclo- propanecarboxamide, as a mixture of trans enantiomers | ¹H NMR (500 MHz, DMSO-d6) δ [ppm] 1.40-1.50 (m, 1H), 1.50-1.57 (m, 1H), 2.11-2.19 (m, 1H), 2.41- 2.47 (m, 1H), 3.59 (s, 3H), 3.74 (s, 3H), 6.55-6.62 (m, 2H), 6.88 (d, J = 8.3 Hz, 1H), 7.20 (d, J = 7.7 Hz, 1H), 7.24-7.31 (m, 2H), 7.34 (t, J = 7.8 Hz, 1H), 7.54 (d, J = 8.5 Hz, 1H), 7.80 (dd, J = 8.5, 2.2 Hz, 1H), 7.93 (d, J = 2.1 Hz, 1H), 10.55 (s, 1H). LCMS (Analytical Method F): Rt = 3.43 mins; MS (ESIPos): m/z = 476 |
| | | | (M + H)⁺ |
| 9 | 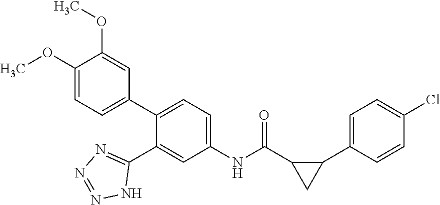 [SMILES: COc1ccc(-c2ccc(NC(=O)C3CC3c3ccc(Cl)cc3)cc2-c2nnn[nH]2)cc1OC] | 2-(4-chlorophenyl)- N-[3′,4′-dimethoxy-2- (1H-tetrazol-5- yl)biphenyl-4-yl]cyclo- propanecarboxamide, as a mixture of trans enantiomers | ¹H NMR (500 MHz, DMSO-d6) δ [ppm] 1.34-1.45 (m, 1H), 1.50-1.57 (m, 1H), 2.06-2.14 (m, 1H), 2.40- 2.46 (m, 1H), 3.58 (s, 3H), 3.74 (s, 3H), 6.54-6.63 (m, 2H), 6.88 (d, J = 8.3 Hz, 1H), 7.24 (d, J = 8.5 Hz, 2H), 7.34-7.40 (m, 2H), 7.54 (d, J = 8.5 Hz, 1H), 7.80 (m, 1H), 7.93 (d, J = 2.1 Hz, 1H), 10.57 (s, 1H). LCMS (Analytical Method F): Rt = 4.34 mins; MS (ESIPos) m/z = 476 (M + H)⁺ |
| 10 | 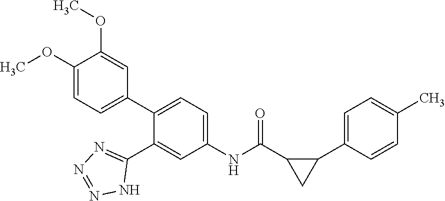 | N-[3′,4′-dimethoxy-2- (1H-tetrazol-5- yl)biphenyl-4-yl]-2-(4- methylphenyl)cyclopro- panecarboxamide, as a mixture of trans enantiomers | ¹H NMR (500 MHz, DMSO-d6) δ [ppm] 1.31-1.38 (m, 1H), 1.43-1.52 (m, 1H), 2.01-2.10 (m, 1H), 2.26 (s, 3H), 2.29-2.39 (m, 1H), 3.57 (s, 3H), 3.72 (s, 3H), 6.51-6.64 (m, 2H), 6.86 (d, J = 8.3 Hz, 1H), 7.08 (m, 4H), 7.52 (d, J = 8.5 Hz, 1H), 7.79 (m, 1H), 7.92 (d, J = 1.7 Hz, 1H), 10.53 (s, 1H). LCMS (Analytical Method F): Rt = 4.34 mins; MS (ESIPos) m/z = 456 (M + H)⁺ |
| 11 | 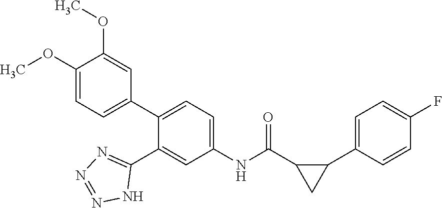 [SMILES: COc1ccc(-c2ccc(NC(=O)C3CC3c3ccc(F)cc3)cc2-c2nnn[nH]2)cc1OC] | N-[3′,4′-dimethoxy-2- (1H-tetrazol-5- yl)biphenyl-4-yl]-2-(4- fluorophenyl)cyclopro- panecarboxamide, as a mixture of trans enantiomers | ¹H NMR (500 MHz, DMSO-d6) δ [ppm] 10.54 (s, 1H), 7.93 (d, J = 2.1 Hz, 1H), 7.80 (dd, J = 8.5, 2.2 Hz, 1H), 7.53 (d, J = 8.5 Hz, 1H), 7.29-7.22 (m, 2H), 7.13 (t, J = 8.8 Hz, 2H), 6.87 (d, J = 8.2 Hz, 1H), 6.61-6.55 (m, 2H), 3.73 (s, 3H), 3.58 (s, 3H), 2.42 (m, 1H), 2.07 (m, 1H), 1.51 (m, 1H), 1.39 (m, 1H). LCMS (Analytical Method F): Rt = 4.08 mins, MS (ESIPos) m/z = 460 (M + H)⁺. |
| 12 | 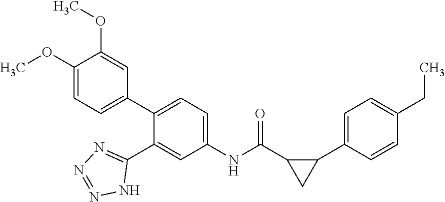 [SMILES: CCc1ccc(C2CC2C(=O)Nc2ccc(-c3ccc(OC)c(OC)c3)c(-c3nnn[nH]3)c2)cc1] | N-[3′,4′-dimethoxy-2- (1H-tetrazol-5- yl)biphenyl-4-yl]-2-(4- ethylphenyl)cyclopro- panecarboxamide, as a mixture of trans enantiomers | ¹H NMR (500 MHz, DMSO-d6) δ [ppm] 10.53 (s, 1H), 7.94 (d, J = 2.1 Hz, 1H), 7.81 (dd, J = 8.5, 2.2 Hz, 1H), 7.54 (d, J = 8.5 Hz, 1H), 7.18- 7.08 (m, 4H), 6.88 (d, J = 8.2 Hz, 1H), 6.61-6.56 (m, 2H), 3.74 (s, 3H), 3.59 (s, 3H), 2.57 (q, J = 7.6 Hz, 2H), 2.40-2.34 (m, 1H), 2.10- 2.03 (m, 1H), 1.53-1.47 (m, 1H), 1.40-1.35 (m, 1H), 1.16 (t, J = 7.6 Hz, 3H). LCMS (Analytical Method D): Rt = |
| | | | 4.43 mins; MS (ESIPos): m/z = 470 |
| | | | (M + H)⁺. |
| 13 | 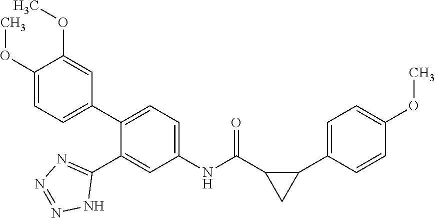 [SMILES: COc1ccc(C2CC2C(=O)Nc2ccc(-c3ccc(OC)c(OC)c3)c(-c3nnn[nH]3)c2)cc1] | N-[3′,4′-dimethoxy-2- (1H-tetrazol-5- yl)biphenyl-4-yl]-2-(4- methoxyphenyl)cyclo- propanecarboxamide, as a mixture of trans enantiomers | ¹H NMR (500 MHz, DMSO-d6) δ [ppm] 10.54 (s, 1H), 7.94 (d, J = 1.8 Hz, 1H), 7.81 (dd, J = 8.5, 2.2 Hz, 1H), 7.55-7.52 (m, 1H), 7.15- 7.10 (m, 2H), 6.91-6.85 (m, 3H), 6.62-6.55 (m, 2H), 3.74 (s, 3H), 3.73 (s, 3H), 3.59 (s, 3H), 2.40-2.33 (m, 1H), 2.06-2.00 (m, 1H), 1.51- 1.44 (m, 1H), 1.38-1.31 (m, 1H). LCMS (Analytical Method D): Rt = 4.05 mins; MS (ESIPos) m/z = 472 (M + H)⁺. |
| 14 | 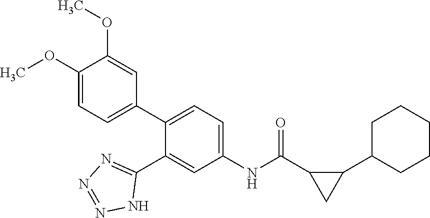 [SMILES: COc1ccc(-c2ccc(NC(=O)C3CC3C3CCCCC3)cc2-c2nnn[nH]2)cc1OC] | 2-Cyclohexyl-N-[3′,4′- dimethoxy-2-(1H- tetrazol-5-yl)biphenyl- 4-yl]cyclopropane- carboxamide, as a single unknown diastereoisomer | ¹H NMR (500 MHz, DMSO-d6) δ [ppm] 0.66-0.86 (m, 2H), 0.90-1.29 (m, 7H), 1.51-1.82 (m, 6H), 3.58 (s, 3H), 3.73 (s, 3H), 6.53-6.61 (m, 2H), 6.87 (d, J = 8.3 Hz, 1H), 7.51 (d, J = 8.5 Hz, 1H), 7.79 (dd, J = 8.5, 2.2 Hz, 1H), 7.91 (d, J = 2.1 Hz, 1H), 10.40 (s, 1H), LCMS (Analytical Method D): Rt = 4.52 mins; MS (ESIPos) m/z = 448 (M + H)⁺. |
| 15 | 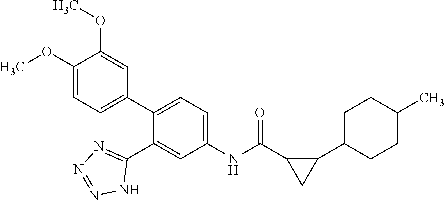 | N-[3′,4′-dimethoxy-2- (1H-tetrazol-5- yl)biphenyl-4-yl]-2-(4- methylcyclohexyl)cyclo- propanecarboxamide, as a single unknown racemic diastereoisomer | ¹H NMR (500 MHz, Methanol-d4) δ [ppm] 0.67-0.84 (m, 2H), 0.84- 1.01 (m, 5H), 1.07-1.25 (m, 4H), 1.25-1.42 (m, 1H), 1.54-1.65 (m, 1H), 1.68-1.78 (m, 2H), 1.78-1.91 (m, 2H), 3.66 (s, 3H), 3.82 (s, 3H), 6.60 (d, J = 2.0 Hz, 1H), 6.69 (dd, J = 8.3, 2.0 Hz, 1H), 6.89 (d, J = 8.3 Hz, 1H), 7.52 (d, J = 8.4 Hz, 1H), 7.81-7.90 (m, 2H), 10.24 (s, 1H). LCMS (Analytical Method F): Rt = |
| | | | 3.86 mins; MS (ESIPos) m/z = 462 |
| | | | (M + H)⁺. |
| 16 | 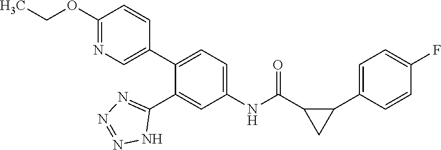 [SMILES: CCOc1ccc(-c2ccc(NC(=O)C3CC3c3ccc(F)cc3)cc2-c2nnn[nH]2)cn1] | N-[4-(6-ethoxypyridin- 3-yl)-3-(1H-tetrazol-5- yl)phenyl]-2-(4- fluorophenyl)cyclopro- panecarboxamide, as a mixture of trans enantiomers | ¹H NMR (500 MHz, DMSO-d6) δ [ppm] 10.59 (s, 1H), 8.03 (s, 1H), 7.90 (d, J = 2.5 Hz, 1H), 7.82 (dd, J = 8.5, 2.1 Hz, 1H), 7.51 (d, J = 8.4 Hz, 1H), 7.33-7.28 (m, 1H), 7.27-7.22 (m, 2H), 7.16-7.10 (m, 2H), 6.70 (d, J = 8.6 Hz, 1H), 4.28 (q, J = 7.0 Hz, 2H), 2.46-2.38 (m, 1H), 2.11-2.03 (m, 1H), 1.56-1.47 |
| | | | (m, 1H), 1.44-1.36 (m, 1H), 1.31 |
| | | | (t, J = 7.0 Hz, 3H). |
| | | | LCMS (Analytical Method D): Rt = |
| | | | 4.20 mins; MS (ESIPos) m/z = |
| | | | 445.1 (M + H)⁺. |
| 17 | 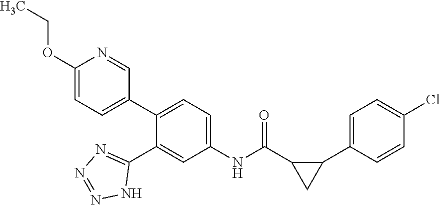 [SMILES: CCOc1ccc(-c2ccc(NC(=O)C3CC3c3ccc(Cl)cc3)cc2-c2nnn[nH]2)cn1] | 2-(4-Chlorophenyl)- N-[4-(6-ethoxypyridin- 3-yl)-3-(1H-tetrazol-5- yl)phenyl]cyclopro- panecarboxamide, as a mixture of trans enantiomers | ¹H NMR (500 MHz, DMSO-d6) δ [ppm] 10.61 (s, 1H), 8.03 (s, 1H), 7.90 (d, J = 2.5 Hz, 1H), 7.82 (dd, J = 8.5, 2.2 Hz, 1H), 7.52 (d, J = 8.4 Hz, 1H), 7.36 (d, J = 8.4 Hz, 2H), 7.31 (d, J = 7.7 Hz, 1H), 7.24 (d, J = 8.5 Hz, 2H), 6.71 (d, J = 8.6 Hz, 1H), 4.29 (q, J = 7.0 Hz, 2H), 2.46-2.39 (m, 1H), 2.11 (m, 1H), 1.54 (m, 1H), 1.45-1.37 (m, 1H), 1.31 (t, J = 7.0 Hz, 3H). LCMS (Analytical Method F): Rt = 3.63 mins; MS (ESIPos) m/z = |
| | | | 461.1 (M + H)⁺. |
| 18 | 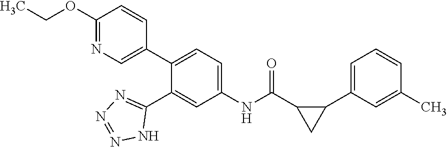 [SMILES: CCOc1ccc(-c2ccc(NC(=O)C3CC3c3cccc(C)c3)cc2-c2nnn[nH]2)cn1] | N-[4-(6-Ethoxypyridin- 3-yl)-3-(1H-tetrazol-5- yl)phenyl]-2-(3- methylphenyl)cyclopro- panecarboxamide, as a mixture of trans enantiomers | ¹H NMR (500 MHz, DMSO-d6) δ [ppm] 10.58 (s, 1H), 8.03 (s, 1H), 7.90 (d, J = 2.2 Hz, 1H), 7.81 (dd, J = 8.5, 2.2 Hz, 1H), 7.51 (d, J = 8.4 Hz, 1H), 7.30 (dd, J = 8.6, 2.5 Hz, 1H), 7.18 (t, J = 7.5 Hz, 1H), 7.05-6.96 (m, 3H), 6.70 (d, J = 8.5 Hz, 1H), 4.28 (q, J = 7.0 Hz, 2H), 2.38-2.33 (m, 1H), 2.28 (s, 3H), |
| | | | 2.11-2.07 (m, 1H), 1.54-1.47 (m, |
| | | | 1H), 1.42-1.37 (m, 1H), 1.30 (t, |
| | | | J = 7.0 Hz, 3H). |
| | | | LCMS (Analytical Method F): Rt = |
| | | | 3.51 mins; MS (ESIPos) m/z = 441 |
| | | | (M + H)⁺. |
| 19 | 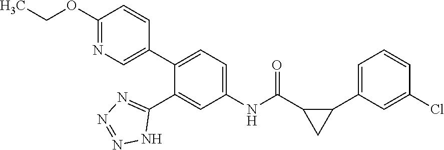 [SMILES: CCOc1ccc(-c2ccc(NC(=O)C3CC3c3cccc(Cl)c3)cc2-c2nnn[nH]2)cn1] | 2-(3-Chlorophenyl)- N-[4-(6-ethoxypyridin- 3-yl)-3-(1H-tetrazol-5- yl)phenyl]cyclopro- panecarboxamide, as a mixture of trans enantiomers | ¹H NMR (500 MHz, DMSO-d6) δ [ppm] 10.59 (s, 1H), 8.05-7.98 (m, 1H), 7.92-7.87 (m, 1H), 7.84-7.77 (m, 1H), 7.54-7.48 (m, 1H), 7.36- 7.24 (m, 4H), 7.23-7.17 (m, 1H), 6.72-6.66 (m, 1H), 4.29 (q, J = 7.0 Hz, 2H), 2.46-2.41 (m, 1H), 2.18- 2.11 (m, 1H), 1.58-1.42 (m, 2H), 1.31 (t, J = 7.0 Hz, 3H). |
| | | | LCMS (Analytical Method F): Rt = |
| | | | 3.56 mins, MS (ESIPos): m/z = 461 |
| | | | (M + H)⁺. |
| 20 | 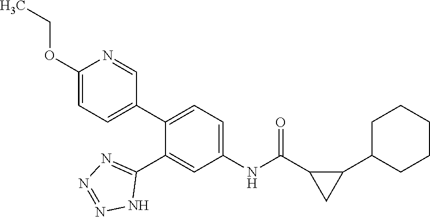 [SMILES: CCOc1ccc(-c2ccc(NC(=O)C3CC3C3CCCCC3)cc2-c2nnn[nH]2)cn1] | 2-Cyclohexyl-N-[4-(6- ethoxypyridin-3-yl)-3- (1H-tetrazol-5- yl)phenyl]cyclopro- panecarboxamide, as a mixture of trans enantiomers | ¹H NMR (500 MHz, DMSO-d6) δ [ppm] 10.44 (s, 1H), 8.00 (s, 1H), 7.88 (d, J = 2.6 Hz, 1H), 7.80 (dd, J = 8.5, 2.2 Hz, 1H), 7.49 (d, J = 8.6 Hz, 1H), 7.29 (dd, J = 8.6, 2.0 Hz, 1H), 6.69 (d, J = 8.6 Hz, 1H), 4.28 (q, J = 7.0 Hz, 2H), 1.83-1.53 (m, 6H), 1.30 (t, J = 7.0 Hz, 3H), 1.22-1.02 (m, 6H), 1.01-0.95 (m, 1H), 0.83-0.70 (m, 2H). LCMS (Analytical Method D): Rt = 4.53 min, MS (ESIPos) m/z = 431.2 (M + H)⁺. |
| 21 | 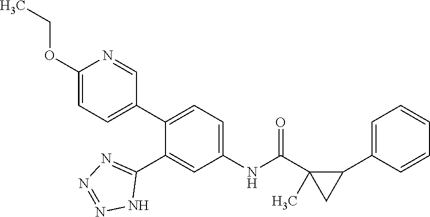 [SMILES: CCOc1ccc(-c2ccc(NC(=O)C3(C)CC3c3ccccc3)cc2-c2nnn[nH]2)cn1] | [4-(6-Ethoxypyridin-3- yl)-3-(1H-tetrazol-5- yl)phenyl]-1-methyl-2- phenylcyclopropane- carboxamide, as a mixture of trans enantiomers | ¹H NMR (500 MHz, DMSO-d6) δ 9.56 (s, 1H), 8.11 (s, 1H), 8.04-7.94 (m, 1H), 7.92 (d, J = 2.5 Hz, 1H), 7.52 (d, J = 8.3 Hz, 1H), 7.37-7.21 (m, 6H), 6.72 (d, J = 8.5 Hz, 1H), 4.29 (q, J = 7.0 Hz, 2H), 2.72-2.65 (m, 1H), 1.68-1.62 (m, 1H), 1.34- 1.26 (m, 4H), 1.11 (s, 3H). LCMS (Analytical Method F): Rt = 3.46 mins; MS ESI(Pos) m/z = 441 (M + H)⁺. |
| 22 | 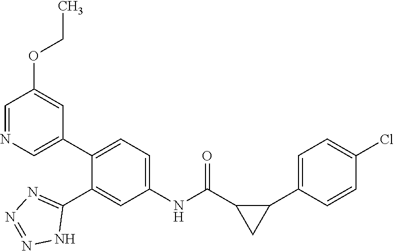 [SMILES: CCOc1cncc(-c2ccc(NC(=O)C3CC3c3ccc(Cl)cc3)cc2-c2nnn[nH]2)c1] | 2-(4-Chlorophenyl)- N-[4-(5-ethoxypyridin- 3-yl)-3-(1H-tetrazol-5- yl)phenyl]cyclopro- panecarboxamide, as a mixture of trans enantiomers | ¹H NMR (500 MHz, DMSO-d6) δ [ppm] 10.64 (s, 1H), 8.19 (d, J = 2.7 Hz, 1H), 8.06 (d, J = 1.7 Hz, 1H), 7.89-7.78 (m, 2H), 7.58 (d, J = 8.5 Hz, 1H), 7.41-7.33 (m, 2H), 7.29-7.19 (m, 2H), 7.05 (s, 1H), 4.01 (q, J = 7.0 Hz, 2H), 2.46-2.40 (m, 1H), 2.15-2.04 (m, 1H), 1.59- 1.50 (m, 1H), 1.48-1.38 (m, 1H), 1.29 (t, J = 7.0 Hz, 3H). LCMS (Analytical Method F): Rt = 2.97 mins; MS (ESIPos) m/z = 461 (M + H)⁺. |
| 23 | 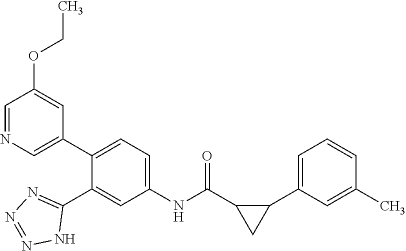 [SMILES: CCOc1cncc(-c2ccc(NC(=O)C3CC3c3cccc(C)c3)cc2-c2nnn[nH]2)c1] | N-[4-(5-Ethoxypyridin- 3-yl)-3-(1H-tetrazol-5- yl)phenyl]-2-(3- methylphenyl)cyclopro- panecarboxamide, as a mixture of trans enantiomers | ¹H NMR (500 MHz, DMSO-d6) δ [ppm] 10.57 (s, 1H), 8.17-8.14 (m, 1H), 8.01 (d, J = 2.1 Hz, 1H), 7.87- 7.80 (m, 2H), 7.52 (d, J = 8.4 Hz, 1H), 7.23-7.14 (m, 1H), 7.06-6.96 (m, 4H), 3.99 (q, J = 7.0 Hz, 2H), 2.40-2.33 (m, 1H), 2.29 (s, 3H), 2.14-2.07 (m, 1H), 1.55-1.47 (m, 1H), 1.43-1.36 (m, 1H), 1.29 (t, J = 7.0 Hz, 3H). LCMS (Analytical Method F): Rt = 2.91 mins; MS (ESIPos) m/z = 441 (M + H)⁺. |
| 24 | 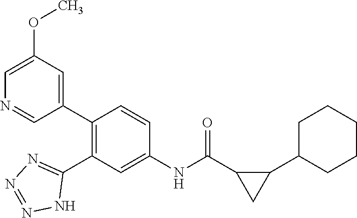 [SMILES: COc1cncc(-c2ccc(NC(=O)C3CC3C3CCCCC3)cc2-c2nnn[nH]2)c1] | 2-Cyclohexyl-N-[4-(5- methoxypyridin-3-yl)- 3-(1H-tetrazol-5- yl)phenyl]cyclopro- panecarboxamide, as a mixture of trans enantiomers | ¹H NMR (500 MHz, DMSO-d6) δ [ppm] 10.46 (s, 1H), 8.17 (d, J = 2.4 Hz, 1H), 8.00 (s, 1H), 7.87-7.79 (m, 2H), 7.53 (d, J = 8.4 Hz, 1H), 7.07- 7.01 (m, 1H), 3.73 (s, 3H), 1.82- 1.56 (m, 6H), 1.25-1.03 (m, 6H), 1.02-0.96 (m, 1H), 0.82-0.71 (m, 2H). LCMS (Analytical Method F): Rt = 2.98 mins; MS (ESIPos) m/z = 419.2 (M + H)⁺. |
| 25 | 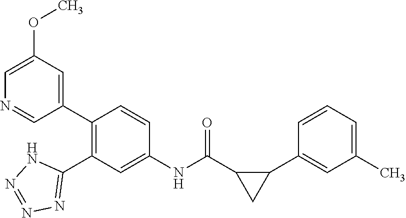 [SMILES: COc1cncc(-c2ccc(NC(=O)C3CC3c3cccc(C)c3)cc2-c2nnn[nH]2)c1] | N-[4-(5-Methoxypyridin- 3-yl)-3-(1H-tetrazol-5- yl)phenyl]-2-(3- methylphenyl)cyclopro- panecarboxamide, as a mixture of trans enantiomers | ¹H NMR (500 MHz, DMSO-d6) δ [ppm] 10.56 (s, 1H), 8.17 (d, J = 2.6 Hz, 1H), 8.00 (d, J = 1.9 Hz, 1H), 7.87-7.79 (m, 2H), 7.52 (d, J = 8.4 Hz, 1H), 7.19 (t, J = 7.5 Hz, 1H), 7.07-6.95 (m, 4H), 3.73 (s, 3H), 2.39-2.34 (m, 1H), 2.29 (s, 3H), 2.14-2.07 (m, 1H), 1.55-1.47 (m, 1H), 1.43-1.36 (m, 1H). LCMS (Analytical Method F): Rt = 2.74 mins; MS (ESIPos) m/z = 427 (M + H)⁺. |
| 26 | 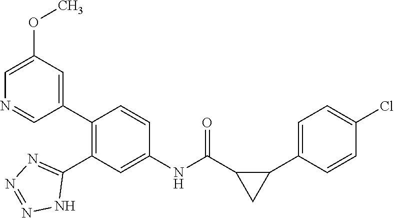 [SMILES: COc1cncc(-c2ccc(NC(=O)C3CC3c3ccc(Cl)cc3)cc2-c2nnn[nH]2)c1] | 2-(4-Chlorophenyl)- N-[4-(5-methoxypyridin- 3-yl)-3-(1H-tetrazol-5- yl)phenyl]cyclopro- panecarboxamide, as a mixture of trans enantiomers | ¹H NMR (500 MHz, DMSO-d6) δ [ppm] 10.60 (s, 1H), 8.16 (d, J = 2.7 Hz, 1H), 7.99 (s, 1H), 7.86-7.79 (m, 2H), 7.52 (d, J = 8.4 Hz, 1H), 7.35 (d, J = 8.4 Hz, 2H), 7.23 (d, J = 8.4 Hz, 2H), 7.10-7.01 (m, 1H), 3.72 (s, 3H), 2.43 (m, 1H), 2.11 (m, 1H), 1.53 (m, 1H), 1.43-1.37 (m, 1H). LCMS (Analytical Method F): Rt = 2.81 min, MS (ESIPos) m/z = 447.1 (M + H)⁺. |
| 27 | 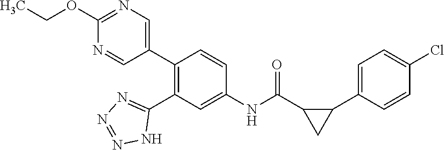 [SMILES: CCOc1ncc(-c2ccc(NC(=O)C3CC3c3ccc(Cl)cc3)cc2-c2nnn[nH]2)cn1] | 2-(4-Chlorophenyl)- N-[4-(2-ethoxypyrimidin- 5-yl)-3-(1H-tetrazol-5- yl)phenyl]cyclopropane- carboxamide, as a mixture of trans enantiomers | ¹H NMR (500 MHz, DMSO-d6) δ [ppm] 10.67 (s, 1H), 8.33 (s, 2H), 8.15 (s, 1H), 7.85 (dd, J = 8.5, 2.2 Hz, 1H), 7.56 (d, J = 8.4 Hz, 1H), 7.36 (d, J = 8.5 Hz, 2H), 7.24 (d, J = 8.5 Hz, 2H), 4.36 (q, J = 7.0 Hz, 2H), 2.44 (ddd, J = 9.6, 6.3, 4.1 Hz, 1H), 2.16-2.09 (m, 1H), 1.58- 1.51 (m, 1H), 1.42 (ddd, J = 8.1, 6.4, 4.3 Hz, 1H), 1.34 (t, J = 7.0 |
| | | | Hz, 3H). |
| | | | LCMS (Analytical Method F): Rt = |
| | | | 3.38 mins; MS (ESIPos) m/z = |
| | | | 462.1 (M + H)⁺. |
| 28 | 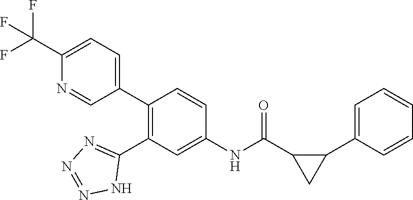 [SMILES: O=C(Nc1ccc(-c2ccc(C(F)(F)F)nc2)c(-c2nnn[nH]2)c1)C1CC1c1ccccc1] | 2-Phenyl-N-{3-(1H- tetrazol-5-yl)-4-[6- (trifluoromethyl)pyridin- 3-yl]phenyl}cyclopro- panecarboxamide, as a mixture of trans enantiomers | ¹H NMR (500 MHz, DMSO-d6) δ [ppm] 10.69 (s, 1H), 8.53 (d, J = 2.0 Hz, 1H), 8.18 (d, J = 1.8 Hz, 1H), 7.88 (dd, J = 8.5, 2.2 Hz, 1H), 7.85 (d, J = 8.1 Hz, 1H), 7.76 (dd, J = 8.1, 1.8 Hz, 1H), 7.62 (d, J = 8.5 Hz, 1H), 7.35-7.27 (m, 2H), 7.25-7.17 (m, 3H), 2.43 (ddd, J = 9.4, 6.3, 4.1 Hz, 1H), 2.17-2.10 (m, 1H), 1.58-1.51 (m, 1H), 1.43 (ddd, J = 8.1, 6.4, 4.2 Hz, 1H). LCMS (Analytical Method F): Rt = 3.42 mins; MS (ESIPos) m/z = |
| | | | 451.1 (M + H)⁺. |
| 29 | 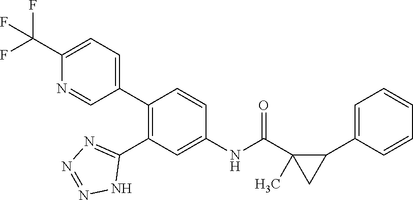 [SMILES: CC1(C(=O)Nc2ccc(-c3ccc(C(F)(F)F)nc3)c(-c3nnn[nH]3)c2)CC1c1ccccc1] | 1-Methyl-2-phenyl-N-{3- (1H-tetrazol-5-yl)-4-[6- (trifluoromethyl)pyridin- 3-yl]phenyl}cyclopro- panecarboxamide, as a mixture of trans enantiomers | ¹H NMR (500 MHz, DMSO-d6) δ [ppm] 9.65 (s, 1H), 8.55 (d, J = 2.0 Hz, 1H), 8.27 (d, J = 1.8 Hz, 1H), 8.06 (dd, J = 8.5, 2.2 Hz, 1H), 7.86 (d, J = 8.1 Hz, 1H), 7.77 (dd, J = 8.1, 1.8 Hz, 1H), 7.63 (d, J = 8.5 Hz, 1H), 7.37-7.31 (m, 2H), 7.26 (dd, J = 16.2, 7.6 Hz, 3H), 2.70 (dd, J = 9.0, 7.2 Hz, 1H), 1.67 (dd, J = 9.1, 4.6 Hz, 1H), 1.31 (dd, J = 6.9, 4.6 Hz, 1H), 1.13 (s, 3H). LCMS (Analytical Method F): Rt = |
| | | | 3.59 mins; MS (ESIPos) m/z = |
| | | | 465.1 (M + H)⁺. |
| 30 | 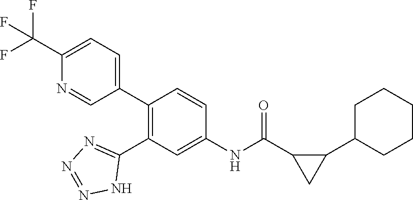 [SMILES: O=C(Nc1ccc(-c2ccc(C(F)(F)F)nc2)c(-c2nnn[nH]2)c1)C1CC1C1CCCCC1] | 2-Cyclohexyl-N-{3-(1H- tetrazol-5-yl)-4-[6- (trifluoromethyl)pyridin- 3-yl]phenyl}cyclopro- panecarboxamide, as a mixture of trans enantiomers | ¹H NMR (500 MHz, DMSO-d6) δ [ppm] 10.54 (s, 1H), 8.52 (d, J = 1.9 Hz, 1H), 8.16 (d, J = 1.7 Hz, 1H), 7.87 (dd, J = 8.5, 2.2 Hz, 1H), 7.85 (d, J = 8.2 Hz, 1H), 7.75 (dd, J = 8.1, 1.7 Hz, 1H), 7.60 (d, J = 8.5 Hz, 1H), 1.82-1.57 (m, 6H), 1.27-0.98 (m, 7H), 0.82-0.73 (m, 2H). LCMS (Analytical Method A): Rt = 1.25 min, MS (ESIPos) m/z = 457 (M + H)⁺. |
| 31 | 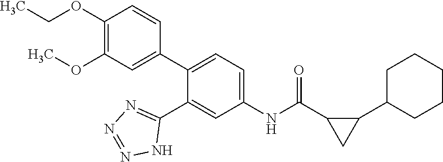 [SMILES: CCOc1ccc(-c2ccc(NC(=O)C3CC3C3CCCCC3)cc2-c2nnn[nH]2)cc1OC] | 2-cyclohexyl-N-[4′- ethoxy-3′-methoxy-2- (1H-tetrazol-5- yl)biphenyl-4- yl]cyclopropane- carboxamide, as a mixture of trans enantiomers | ¹H NMR (500 MHz, DMSO-d6) δ [ppm] 0.70-0.83 (m, 2H), 0.93-1.02 (m, 1H), 1.02-1.25 (m, 6H), 1.31 (t, J = 7.0 Hz, 3H), 1.56-1.65 (m, 2H), 1.65-1.82 (m, 4H), 3.58 (s, 3H), 3.98 (q, J = 7.0 Hz, 2H), 6.50-6.62 (m, 2H), 6.85 (d, J = 8.2 Hz, 1H), 7.51 (d, J = 8.5 Hz, 1H), 7.79 (dd, J = 8.5, 2.2 Hz, 1H), 7.90 (d, J = 1.9 Hz, 1H), 10.39 (s, 1H). |
| | | | LCMS (Analytical Method F): Rt = |
| | | | 3.80 mins; MS (ESIpos) m/z = 462 |
| | | | (M + H)⁺. |
| 32 | 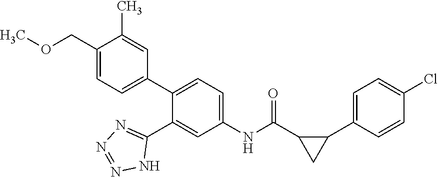 [SMILES: COCc1ccc(-c2ccc(NC(=O)C3CC3c3ccc(Cl)cc3)cc2-c2nnn[nH]2)cc1C] | 2-(4-chlorophenyl)-N-[4′- (methoxymethyl)-3′- methyl-2-(1H-tetrazol-5- yl)biphenyl-4-yl]cyclopro- panecarboxamide, as a mixture of trans enantiomers | ¹H NMR (500 MHz, DMSO-d6) δ [ppm] 1.33-1.48 (m, 1H), 1.49-1.60 (m, 1H), 2.05-2.15 (m, 1H), 2.20 (s, 3H), 2.39-2.46 (m, 1H), 3.30 (s, 3H), 4.37 (s, 2H), 6.73-6.78 (m, 1H), 6.91-6.95 (m, 1H), 7.16 (d, J = 7.9 Hz, 1H), 7.21-7.27 (m, 2H), 7.30-7.39 (m, 2H), 7.49 (d, J = 8.5 Hz, 1H), 7.81 (dd, J = 8.5, 2.1 Hz, 1H), 7.95 (s, 1H), 10.57 (s, 1H). LCMS (Analytical Method F): Rt = |
| | | | 3.72 mins; MS (ESIPos) m/z = |
| | | | 474.2 (M + H)⁺. |
| 33 | 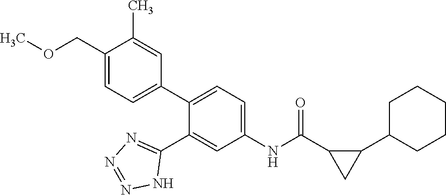 [SMILES: COCc1ccc(-c2ccc(NC(=O)C3CC3C3CCCCC3)cc2-c2nnn[nH]2)cc1C] | 2-cyclohexyl-N-[4′- (methoxymethyl)-3′- methyl-2-(1H-tetrazol-5- yl)biphenyl-4-yl]cyclo- propanecarboxamide, as a mixture of trans enantiomers | ¹H NMR (500 MHz, DMSO-d6) δ [ppm] 0.69-0.88 (m, 2H), 0.94-1.32 (m, 7H), 1.51-1.84 (m, 6H), 2.20 (s, 3H), 3.30 (s, 3H), 4.37 (s, 2H), 6.72-6.78 (m, 1H), 6.90-6.95 (m, 1H), 7.16 (d, J = 7.9 Hz, 1H), 7.48 (d, J = 8.4 Hz, 1H), 7.80 (dd, J = 8.5, 2.2 Hz, 1H), 7.91-7.96 (m, 1H), 10.42 (s, 1H). LCMS (Analytical Method F): Rt = 3.91 mins; MS (ESIPos) m/z = 446.3 (M + H)⁺. |
| 34 | 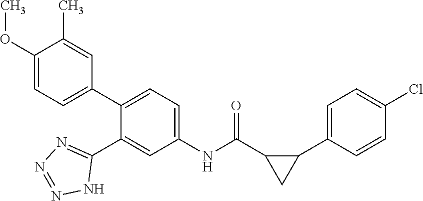 [SMILES: COc1ccc(-c2ccc(NC(=O)C3CC3c3ccc(Cl)cc3)cc2-c2nnn[nH]2)cc1C] | 2-(4-chlorophenyl)-N-[4′- methoxy-3′-methyl-2-(1H- tetrazol-5-yl)biphenyl-4- yl]cyclopropanecarbox- amide, as a mixture of trans enantiomers | ¹H NMR (500 MHz, DMSO-d6) δ [ppm] 10.54 (s, 1H), 7.91 (d, J = 2.0 Hz, 1H), 7.79 (dd, J = 8.5, 2.3 Hz, 1H), 7.46 (d, J = 8.5 Hz, 1H), 7.36 (d, J = 8.5 Hz, 2H), 7.24 (d, J = 8.5 Hz, 2H), 6.90 (d, J = 1.7 Hz, 1H), 6.82 (d, J = 8.6 Hz, 1H), 6.76 (dd, J = 8.3, 2.2 Hz, 1H), 3.76 (s, 3H), 2.42 (ddd, J = 9.5, 6.4, 4.1 Hz, 1H), 2.13-2.09 (m, 1H), 2.08 (s, 3H), 1.53 (dt, J = 9.4, 4.4 Hz, 1H), 1.43-1.38 (m, 1H). LCMS (Analytical Method D): Rt = |
| | | | 4.54 mins; MS (ESIPos) m/z = |
| | | | 460.1 (M + H)⁺. |
| 35 | 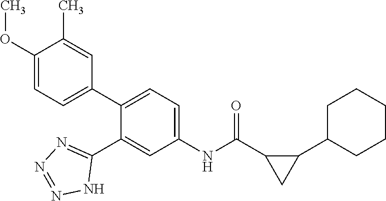 [SMILES: COc1ccc(-c2ccc(NC(=O)C3CC3C3CCCCC3)cc2-c2nnn[nH]2)cc1C] | 2-Cyclohexyl-N-[4′- methoxy-3′-methyl-2-(1H- tetrazol-5-yl)biphenyl-4- yl]cyclopropanecarbox- amide, as a single unknown diastereoisomer | ¹H NMR (500 MHz, DMSO-d6) δ [ppm] 10.33 (s, 1H), 7.86-7.69 (m, 2H), 7.43-7.30 (m, 1H), 6.88 (s, 1H), 6.83-6.73 (m, 2H), 3.75 (s, 3H), 2.06 (s, 3H), 1.81-1.64 (m, 4H), 1.64-1.56 (m, 2H), 1.25-1.01 (m, 6H), 1.01-0.94 (m, 1H), 0.81- 0.69 (m, 2H). LCMS (Analytical Method F): Rt = 4.07 min; MS (ESIpos) m/z = 432.2 (M + H)⁺. |
| 36 | 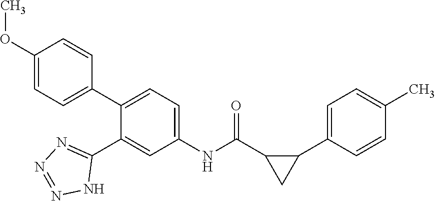 [SMILES: COc1ccc(-c2ccc(NC(=O)C3CC3c3ccc(C)cc3)cc2-c2nnn[nH]2)cc1] | N-[4′-methoxy-2-(1H- tetrazol-5-yl)biphenyl-4- yl]-2-(4- methylphenyl)cyclopro- panecarboxamide, as a mixture of trans enantiomers | ¹H NMR (500 MHz, DMSO-d6) δ [ppm] 1.32-1.39 (m, 1H), 1.43- 1.54 (m, 1H), 2.01-2.09 (m, 1H), 2.27 (s, 3H), 2.29-2.39 (m, 1H), 3.73 (s, 3H), 6.80-6.88 (m, 2H), 6.92-7.01 (m, 2H), 7.06-7.14 (m, 4H), 7.46 (d, J = 8.4 Hz, 1H), 7.79 (dd, J = 8.5, 2.2 Hz, 1H), 7.93 (s, 1H), 10.52 (s, 1H). LCMS (Analytical Method F): Rt = 3.58 mins; MS (ESIPos) m/z = 426.2 (M + H)⁺. |
| 37 | 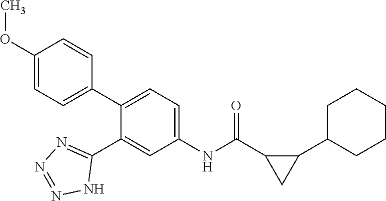 [SMILES: COc1ccc(-c2ccc(NC(=O)C3CC3C3CCCCC3)cc2-c2nnn[nH]2)cc1] | 2-cyclohexyl-N-[4′- methoxy-2-(1H-tetrazol-5- yl)biphenyl-4- yl]cyclopropanecarbox- amide, as a racemate of an unknown diastereoisomer | ¹H NMR (500 MHz, DMSO-d6) δ [ppm] 0.71-0.82 (m, 2H), 0.95-1.24 (m, 7H), 1.57-1.82 (m, 6H), 3.73 (s, 3H), 6.85 (d, J = 8.8 Hz, 2H), 6.96 (d, J = 8.8 Hz, 2H), 7.45 (d, J = 8.4 Hz, 1H), 7.78 (dd, J = 8.5, 2.2 Hz, 1H), 7.91 (s, 1H), 10.39 (s, 1H). LCMS (Analytical Method F): Rt = 3.82 mins; MS (ESIPos) m/z = 418.2 (M + H)⁺ |
| 38 | 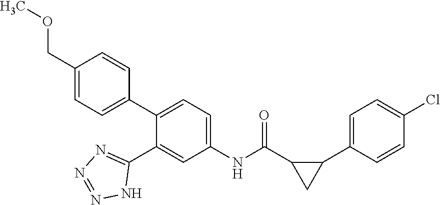 [SMILES: COCc1ccc(-c2ccc(NC(=O)C3CC3c3ccc(Cl)cc3)cc2-c2nnn[nH]2)cc1] | 2-(4-chlorophenyl)- N-[4′-(methoxymethyl)-2- (1H-tetrazol-5- yl)biphenyl-4- yl]cyclopropanecarbox- amide, as a mixture of trans enantiomers | ¹H NMR (500 MHz, DMSO-d6) δ [ppm] 10.58 (s, 1H), 7.96 (s, 1H), 7.81 (dd, J = 8.5, 2.1 Hz, 1H), 7.50 (d, J = 8.5 Hz, 1H), 7.36 (d, J = 8.3 Hz, 2H), 7.23 (t, J = 7.7 Hz, 4H), 7.02 (d, J = 8.0 Hz, 2H), 4.38 (s, 2H), 3.28 (s, 3H), 2.45-2.39 (m, 1H), 2.10 (dt, J = 8.8, 5.0 Hz, 1H), 1.53 (dt, J = 9.2, 4.7 Hz, 1H), 1.45- 1.33 (m, 1H). LCMS (Analytical Method F): Rt = 3.63 min; MS (ESIPos) m/z = 461.1 (M + H)⁺. |
| 39 | 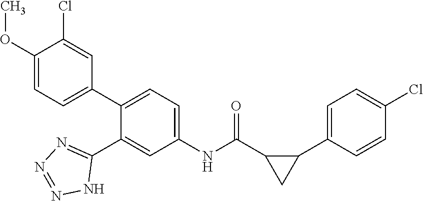 [SMILES: COc1ccc(-c2ccc(NC(=O)C3CC3c3ccc(Cl)cc3)cc2-c2nnn[nH]2)cc1Cl] | N-[3′-chloro-4′-methoxy- 2-(1H-tetrazol-5- yl)biphenyl-4-yl]-2-(4- chlorophenyl)cyclopro- panecarboxamide, as a mixture of trans enantiomers | ¹H NMR (500 MHz, DMSO-d6) δ [ppm] 10.57 (s, 1H), 7.95 (d, J = 1.8 Hz, 1H), 7.79 (dd, J = 8.5, 2.2 Hz, 1H), 7.47 (d, J = 8.5 Hz, 1H), 7.35 (d, J = 8.4 Hz, 2H) 7.23 (d, J = 8.5 Hz, 2H), 7.14 (d, J = 2.2 Hz, 1H), 7.03 (d, J = 8.6 Hz, 1H), 6.90 (dd, J = 8.5, 2.2 Hz, 1H), 3.83 (s, 3H), 2.42 (ddd, J = 9.5, 6.3, 4.1 Hz, 1H), 2.09 (dt, J = 8.7, 4.8 Hz, 1H), 1.52 (dt, J = 9.3, 4.7 Hz, 1H), 1.44-1.36 (m, 1H). |
| | | | LCMS (Analytical Method F): Rt = |
| | | | 3.78 min; MS (ESIPos) m/z = |
| | | | 480.1 (M + H)⁺. |
| 40 | 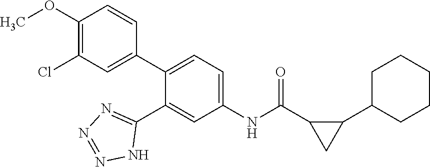 [SMILES: COc1ccc(-c2ccc(NC(=O)C3CC3C3CCCCC3)cc2-c2nnn[nH]2)cc1Cl] | N-[3′-chloro-4′-methoxy- 2-(1H-tetrazol-5- yl)biphenyl-4-yl]-2- cyclohexylcyclopropane- carboxamide, as a racemate of an unknown diastereoisomer | ¹H NMR (500 MHz, DMSO-d6) δ [ppm] 10.43 (s, 1H), 7.96 (d, J = 2.2 Hz, 1H), 7.79 (dd, J = 8.5, 2.2 Hz, 1H), 7.48 (d, J = 8.5 Hz, 1H), 7.15 (d, J = 2.2 Hz, 1H), 7.05 (d, J = 8.7 Hz, 1H), 6.89 (dd, J = 8.5, 2.2 Hz, 1H), 3.84 (s, 3H), 1.83- 1.65 (m, 4H), 1.62 (dt, J = 8.2, 4.4 Hz, 2H), 1.23-1.02 (m, 6H), 0.99 (dt, J = 8.5, 3.9 Hz, 1H), 0.83-0.70 |
| | | | (m, 2H). |
| | | | LCMS (Analytical Method F): Rt = |
| | | | 1.23 min; MS (ESIPos) m/z = |
| | | | 452.3 (M + H)⁺. |
| 41 | 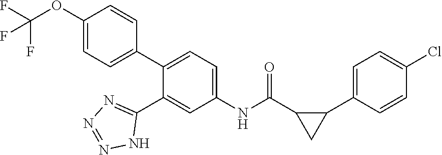 [SMILES: O=C(Nc1ccc(-c2ccc(OC(F)(F)F)cc2)c(-c2nnn[nH]2)c1)C1CC1c1ccc(Cl)cc1] | 2-(4-chlorophenyl)-N-[2- (1H-tetrazol-5-yl)-4′- (trifluoro- methoxy)biphenyl-4- yl]cyclopropanecarbox- amide, as a mixture of trans enantiomers | ¹H NMR (500 MHz, Methanol-d4) δ [ppm] 1.38-1.47 (m, 1H), 1.61- 1.72 (m, 1H), 2.04-2.13 (m, 1H), 2.46-2.57 (m, 1H), 7.17-7.26 (m, 6H), 7.28-7.35 (m, 2H), 7.55 (d, J = 8.5 Hz, 1H), 7.90 (dd, J = 8.5, 2.2 Hz, 1H), 8.00 (d, J = 2.2 Hz, 1H). LCMS (Analytical Method F): Rt = 3.99 mins; MS (ESIPos) m/z = 500 |
| | | | (M + H)⁺. |
| 42 | 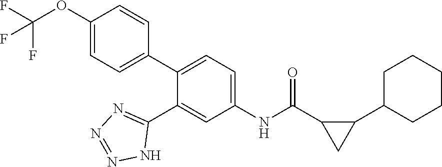 [SMILES: O=C(Nc1ccc(-c2ccc(OC(F)(F)F)cc2)c(-c2nnn[nH]2)c1)C1CC1C1CCCCC1] | 2-cyclohexyl-N-[2-(1H- tetrazol-5-yl)-4′-(trifluoro- methoxy)biphenyl-4- yl]cyclopropanecarbox- amide, as a racemate of an unknown diastereoisomer | ¹H NMR (500 MHz, Methanol-d4) δ [ppm] 0.71-0.84 (m, 2H), 1.06- 1.36 (m, 7H), 1.54-1.90 (m, 6H), 7.15-7.24 (m, 4H), 7.51 (d, J = 8.5 Hz, 1H), 7.83-7.89 (m, 1H), 7.94 (t, J = 2.0 Hz, 1H), 10.29 (s, 1H). LCMS (Analytical Method F): Rt = 4.18 mins; MS (ESIPos) m/z = 472 (M + H)⁺. |
| 43 | 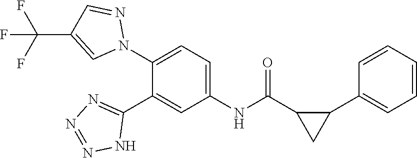 [SMILES: O=C(Nc1ccc(-n2cc(C(F)(F)F)cn2)c(-c2nnn[nH]2)c1)C1CC1c1ccccc1] | 2-Phenyl-N-{3-(1H- tetrazol-5-yl)-4-[4- (trifluoromethyl)-1H- pyrazol-1- yl]phenyl}cyclopro- panecarboxamide, as a mixture of trans enantiomers | ¹H NMR (500 MHz, DMSO-d6) δ [ppm] 10.74 (s, 1H), 8.71 (s, 1H), 8.20 (s, 1H), 7.96 (s, 1H), 7.89 (dd, J = 8.8, 2.4 Hz, 1H), 7.68 (d, J = 8.7 Hz, 1H), 7.34-7.28 (m, 2H), 7.25-7.18 (m, 3H), 2.43 (ddd, J = 9.4, 6.4, 4.1 Hz, 1H), 2.16-2.09 (m, 1H), 1.59-1.51 (m, 1H), 1.44 (ddd, J = 8.1, 6.4, 4.2 Hz, 1H). LCMS (Analytical Method F): Rt = |
| | | | 3.36 mins; MS (ESIPos) m/z = |
| | | | 440.1 (M + H)⁺. |
| 44 | 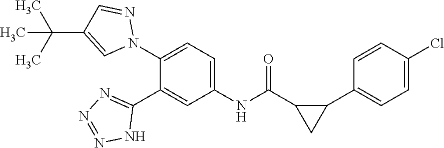 [SMILES: CC(C)(C)c1cnn(-c2ccc(NC(=O)C3CC3c3ccc(Cl)cc3)cc2-c2nnn[nH]2)c1] | N-[4-(4-tert-butyl-1H- pyrazol-1-yl)-3-(1H- tetrazol-5-yl)phenyl]-2-(4- chlorophenyl)cyclopro- panecarboxamide, as a mixture of trans enantiomers, | ¹H NMR (500 MHz, DMSO-d6) δ [ppm] 10.64 (s, 1H), 7.98 (s, 1H), 7.86 (dd, J = 8.8, 2.4 Hz, 1H), 7.69- 7.58 (m, 2H), 7.44 (d, J = 0.6 Hz, 1H), 7.39-7.32 (m, 2H), 7.28-7.20 (m, 2H), 2.43 (ddd, J = 9.3, 6.3, 4.1 Hz, 1H), 2.13-2.03 (m, 1H), 1.58- 1.49 (m, 1H), 1.42 (ddd, J = 8.2, 6.4, 4.3 Hz, 1H), 1.20 (s, 9H). |
| | | | LCMS (Analytical Method F): Rt = |
| | | | 3.82 mins; MS (ESIPos) m/z = |
| | | | 462.1 (M + H)⁺. |
| 45 | 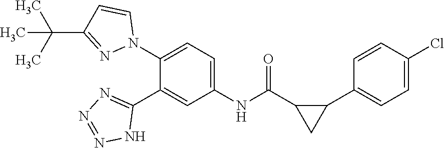 [SMILES: CC(C)(C)c1ccn(-c2ccc(NC(=O)C3CC3c3ccc(Cl)cc3)cc2-c2nnn[nH]2)n1] | N-[4-(3-tert-butyl-1H- pyrazol-1-yl)-3-(1H- tetrazol-5-yl)phenyl]-2-(4- chlorophenyl)cyclopro- panecarboxamide, as a mixture of trans enantiomers | ¹H NMR (500 MHz, DMSO-d6) δ [ppm] 10.63 (s, 1H), 7.94 (s, 1H), 7.88 (s, 1H), 7.86 (dd, J = 8.8, 2.4 Hz, 1H), 7.65 (d, J = 8.8 Hz, 1H), 7.40-7.32 (m, 2H), 7.28-7.21 (m, 2H), 6.28 (d, J = 2.4 Hz, 1H), 2.43 (ddd, J = 9.4, 6.3, 4.1 Hz, 1H), 2.13-2.04 (m, 1H), 1.57-1.49 (m, 1H), 1.46-1.35 (m, 1H), 1.04 (s, |
| | | | 9H). |
| | | | LCMS (Analytical Method F): Rt = |
| | | | 3.80 mins; MS (ESIPos) m/z = |
| | | | 462.2 (M + H)⁺. |
| 46 | 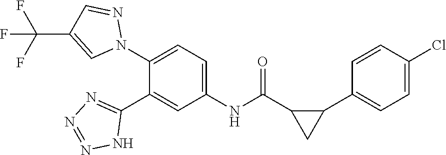 [SMILES: O=C(Nc1ccc(-n2cc(C(F)(F)F)cn2)c(-c2nnn[nH]2)c1)C1CC1c1ccc(Cl)cc1] | 2-(4-Chlorophenyl)-N-{3- (1H-tetrazol-5-yl)-4-[4- (trifluoromethyl)-1H- pyrazol-1- yl]phenyl}cyclopropane- carboxamide, as a mixture of trans enantiomers | ¹H NMR (500 MHz, DMSO-d6) δ [ppm] 10.76 (s, 1H), 8.71 (s, 1H), 8.19 (s, 1H), 7.97 (s, 1H), 7.90 (dd, J = 8.8, 2.4 Hz, 1H), 7.69 (d, J = 8.7 Hz, 1H), 7.39-7.33 (m, 2H), 7.28-7.22 (m, 2H), 2.45 (ddd, J = 9.5, 6.4, 4.1 Hz, 1H), 2.15-2.07 (m, 1H), 1.59-1.52 (m, 1H), 1.44 (ddd, J = 8.1, 6.4, 4.3 Hz, 1H). LCMS (Analytical Method F): Rt = |
| | | | 3.61 mins; MS (ESIPos) m/z = 474 |
| | | | (M + H)⁺. |
| 47 | 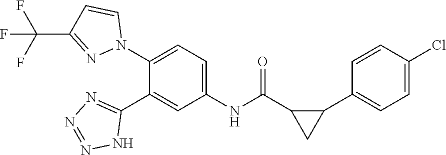 [SMILES: O=C(Nc1ccc(-n2ccc(C(F)(F)F)n2)c(-c2nnn[nH]2)c1)C1CC1c1ccc(Cl)cc1] | 2-(4-Chlorophenyl)-N-{3- (1H-tetrazol-5-yl)-4- [3-(trifluoromethyl)-1H- pyrazol-1- yl]phenyl}cyclopropane- carboxamide, as a mixture of trans enantiomers | ¹H NMR (500 MHz, DMSO-d6) δ [ppm] 10.77 (s, 1H), 8.20 (s, 1H), 8.18-8.14 (m, 1H), 7.90 (dd, J = 8.8, 2.4 Hz, 1H), 7.71 (d, J = 8.7 Hz, 1H), 7.39-7.34 (m, 2H), 7.28- 7.23 (m, 2H), 6.88 (d, J = 2.4 Hz, 1H), 2.47-2.42 (m, 1H), 2.15-2.08 (m, 1H), 1.59-1.52 (m, 1H), 1.44 (ddd, J = 8.2, 6.4, 4.3 Hz, 1H). LCMS (Analytical Method F): Rt = |
| | | | 3.61 mins; MS (ESIPos) m/z = 474 |
| | | | (M + H)⁺. |
| 48 | 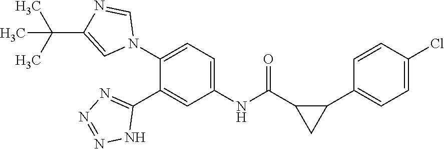 [SMILES: CC(C)(C)c1cn(-c2ccc(NC(=O)C3CC3c3ccc(Cl)cc3)cc2-c2nnn[nH]2)cn1] | N-[4-(4-tert-butyl-1H- imidazol-1-yl)-3-(1H- tetrazol-5-yl)phenyl]-2- (4-chlorophenyl)cyclo- propanecarboxamide, as a mixture of trans enantiomers | ¹H NMR (500 MHz, DMSO-d6) δ [ppm] 10.70 (s, 1H), 8.59 (s, 1H), 8.25 (d, J = 2.4 Hz, 1H), 7.87 (dd, J = 8.7, 2.4 Hz, 1H), 7.55 (d, J = 8.7 Hz, 1H), 7.39-7.34 (m, 2H), 7.27-7.21 (m, 2H), 7.14 (s, 1H), 2.44 (ddd, J = 9.5, 6.3, 4.1 Hz, 1H), 2.17-2.08 (m, 1H), 1.59-1.50 (m, 1H), 1.42 (ddd, J = 8.2, 6.4, 4.3 Hz, |
| | | | 1H), 1.25 (s, 9H). |
| | | | LCMS (Analytical Method F): Rt = |
| | | | 2.68 mins; MS (ESIPos) m/z = |
| | | | 462.1 (M + H)⁺. |
| 49 | 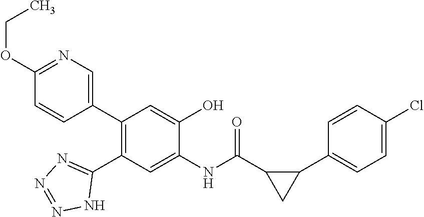 [SMILES: CCOc1ccc(-c2cc(O)c(NC(=O)C3CC3c3ccc(Cl)cc3)cc2-c2nnn[nH]2)cn1] | 2-(4-chlorophenyl)-N-[4- (6-ethoxypyridin-3-yl)-2- hydroxy-5-(1H-tetrazol-5- yl)phenyl]cyclopropane- carboxamide, as a mixture of cis enantiomers | ¹H NMR (400 MHz, DMSO-d6) δ [ppm] 1.30 (t, 3H), 1.32-1.38 (m, 1H), 1.57-1.61 (m, 1H), 2.53-2.59 (m, 2H), 4.28 (q, 2H), 6.68 (d, 1H), 6.91 (s, 1H), 7.24-7.27 (m, 5H), 7.85 (d, 1H), 7.99 (s, 1H), 9.70 (s, 1H), 10.59 (s, 1H). LCMS (method 1): Rt = 1.11; MS (ESIpos) m/z = 477 (M + H)⁺ |
| 50 | 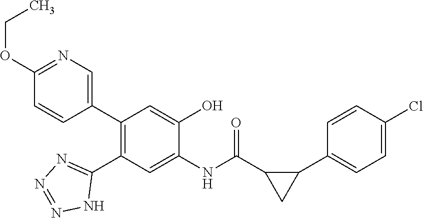 [SMILES: CCOc1ccc(-c2cc(O)c(NC(=O)C3CC3c3ccc(Cl)cc3)cc2-c2nnn[nH]2)cn1] | 2-(4-chlorophenyl)-N-[4- (6-ethoxypyridin-3-yl)-2- hydroxy-5-(1H-tetrazol-5- yl)phenyl]cyclopropane- carboxamide, as a mixture of trans enantiomers | ¹H NMR (400 MHz, DMSO-d6) δ [ppm] 1.29-1.35 (m, 4H), 1.47-1.51 (m, 1H), 2.37-2.42 (m, 1H), 2.57- 2.62 (m, 1H), 4.29 (q, 2H), 6.71 (d, 1H), 6.96 (s, 1H), 7.23 (d, 2H), 7.30 (dd, 1H), 7.37 (d, 2H), 7.89 (d, 1H), 8.29 (s, 1H), 9.82 (s, 1H), 10.74 (s, 1H). LCMS (method 2): Rt = 1.16; MS (ESIpos) m/z = 477 (M + H)⁺ |
| 51 | 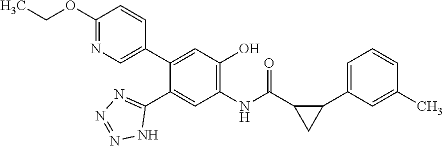 [SMILES: CCOc1ccc(-c2cc(O)c(NC(=O)C3CC3c3cccc(C)c3)cc2-c2nnn[nH]2)cn1] | N-[4-(6-Ethoxypyridin- 3-yl)-2-hydroxy-5-(1H- tetrazol-5-yl)phenyl]-2-(3- methylphenyl)cyclopro- panecarboxamide, as a mixture of trans enantiomers | ¹H NMR (500 MHz, DMSO-d6) δ [ppm] 10.74 (s, 1H), 9.79 (s, 1H), 8.29 (s, 1H), 7.88 (d, J = 2.5 Hz, 1H), 7.31 (dd, J = 8.5, 2.4 Hz, 1H), 7.18 (t, J = 7.5 Hz, 1H), 7.03-6.94 (m, 4H), 6.71 (d, J = 8.6 Hz, 1H), 4.29 (q, J = 7.0 Hz, 2H), 2.60-2.53 (m, 1H), 2.36-2.30 (m, 1H), 2.28 (s, 3H), 1.50-1.42 (m, 1H), 1.37- |
| | | | 1.23 (m, 4H) |
| | | | LCMS (Analytical Method D): Rt = |
| | | | 4.14 mins; MS (ESIpos) m/z = |
| | | | 457.3 (M + H)⁺. |
| 52 | 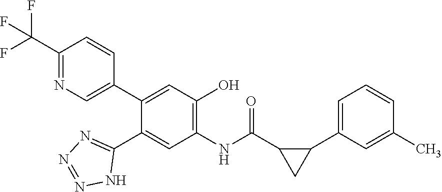 [SMILES: Cc1cccc(C2CC2C(=O)Nc2cc(-c3nnn[nH]3)c(-c3ccc(C(F)(F)F)nc3)cc2O)c1] | N-{2-Hydroxy-5-(1H- tetrazol-5-yl)-4-[6- (trifluoromethyl)pyridin- 3-yl]phenyl}-2-(3- methylphenyl)cyclopro- panecarboxamide, as a mixture of trans enantiomers | ¹H NMR (500 MHz, DMSO-d6) δ [ppm] 10.92 (s, 1H), 9.86 (s, 1H), 8.52 (d, J = 1.9 Hz, 1H), 8.45 (s, 1H), 7.85 (d, J = 8.1 Hz, 1H), 7.77 (dd, J = 8.1, 1.9 Hz, 1H), 7.22-7.15 (m, 1H), 7.04 (s, 1H), 7.03-6.96 (m, 3H), 2.62-2.56 (m, 1H), 2.38-2.31 (m, 1H), 2.29 (s, 3H), 1.52-1.43 (m, 1H), 1.36-1.27 (m, 1H) LCMS (Analytical Method D): Rt = 4.28 mins; MS (ESIpos) m/z = 481.05 (M + H)⁺. |
| 53 | 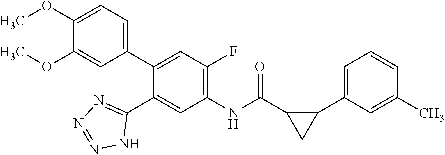 | N-[5-Fluoro-3′,4′- dimethoxy-2-(1H-tetrazol- 5-yl)biphenyl-4-yl]-2-(3- methylphenyl)cyclopro- panecarboxamide, as a mixture of trans enantiomers | ¹H NMR (500 MHz, DMSO-d6) δ [ppm] 10.31 (s, 1H), 8.31 (d, J = 7.9 Hz, 1H), 7.53 (d, J = 11.6 Hz, 1H), 7.18 (t, J = 7.5 Hz, 1H), 7.04- 6.95 (m, 3H), 6.88 (d, J = 8.4 Hz, 1H), 6.64 (d, J = 2.1 Hz, 1H), 6.59 (dd, J = 8.3, 2.1 Hz, 1H), 3.74, (s, 3H), 3.60 (s, 3H), 2.42 (dt, J = 8.6, 5.1 Hz, 1H), 2.38-2.34 (m, 2H), 2.29, (s, 3H), 1.48 (dt, J = 9.2, 4.3 |
| | | | Hz, 1H), 1.36 (ddd, J = 8.0, 6.3, 4.1 |
| | | | Hz, 1H). |
| | | | LCMS (Analytical Method F): Rt = |
| | | | 3.46 mins; MS (ESIpos) m/z = |
| | | | 474.2 (M + H)⁺. |
| 54 | 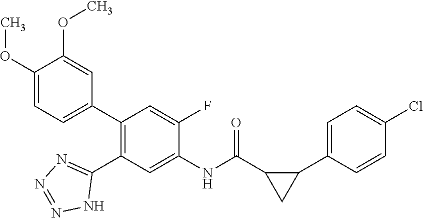 [SMILES: COc1ccc(-c2cc(F)c(NC(=O)C3CC3c3ccc(Cl)cc3)cc2-c2nnn[nH]2)cc1OC] | 2-(4-Chlorophenyl)-N-[5- fluoro-3′,4′-dimethoxy- 2-(1H-tetrazol-5- yl)biphenyl-4-yl]cyclopro- panecarboxamide, as a mixture of trans enantiomers | ¹H NMR (400 MHz, DMSO-d6) δ [ppm] 1.36-1.41 (m, 1H), 1.49-1.51 (m, 1H), 1.41-1.45 (m, 2H), 3.59 (s, 3H), 3.74 (s, 3H), 6.60-6.64 (m, 2H), 6.87 (d, 1H), 7.24 (d, 2H), 7.37 (d, 2H), 7.50 (d, 1H), 8.26 (d, 1H), 10.31 (s, 1H). LCMS (method 1): Rt = 1.19; MS (ESIpos) m/z = 494 (M + H)⁺. |
| 55 | 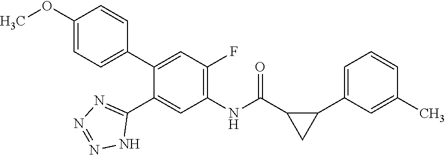 | N-[5-Fluoro-4′-methoxy- 2-(1H-tetrazol-5- yl)biphenyl-4-yl]-2-(3- methylphenyl)cyclopro- panecarboxamide, as a mixture of trans enantiomers | ¹H NMR (500 MHz, DMSO-d6) δ [ppm] 10.30 (s, 1H), 8.32 (d, J = 7.8 Hz, 1H), 7.46 (d, J = 11.7 Hz, 1H), 7.21-7.16 (m, 1H), 7.04-6.96 (m, 5H), 6.89-6.83 (m, 2H), 3.74 (s, 3H), 2.45-2.39 (m, 1H), 2.38- 2.32 (m, 1H), 2.29 (s, 3H), 1.53- 1.43 (m, 1H), 1.40-1.31 (m, 1H). LCMS (Analytical Method D): Rt = 4.43 mins; MS (ESIpos) m/z = |
| | | | 444.05 (M + H)⁺. |
| 56 | 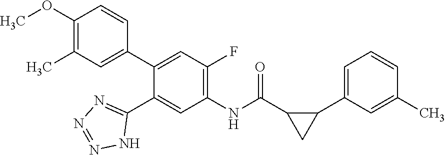 | N-[5-Fluoro-4′-methoxy- 3′-methyl-2-(1H-tetrazol- 5-yl)biphenyl-4-yl]-2-(3- methylphenyl)cyclopro- panecarboxamide, as a mixture of trans enantiomers isomer | ¹H NMR (500 MHz, DMSO-d6) δ [ppm] 10.28 (s, 1H), 8.29 (d, J = 7.8 Hz, 1H), 7.43 (d, J = 11.7 Hz, 1H), 7.21-7.15 (m, 1H), 7.04-6.96 (m, 3H), 6.96-6.92 (m, 1H), 6.83 (d, J = 8.5 Hz, 1H), 6.79 (dd, J = 8.4, 2.0 Hz, 1H), 3.76 (s, 3H), 2.44- 2.38 (m, 1H), 2.38-2.32 (m, 1H), 2.28 (s, 3H), 2.08 (s, 3H), 1.55-1.42 (m, 1H), 1.41-1.30 (m, 1H). |
| | | | LCMS (Analytical Method D): Rt = |
| | | | 4.59 mins; MS (ESIpos) m/z = |
| | | | 458.1 (M + H)⁺. |
| 57 | 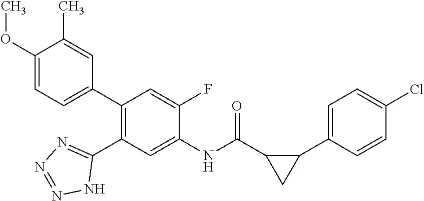 [SMILES: COc1ccc(-c2cc(F)c(NC(=O)C3CC3c3ccc(Cl)cc3)cc2-c2nnn[nH]2)cc1C] | 2-(4-chlorophenyl)-N-[5- fluoro-4′-methoxy-3′- methyl-2-(1H-tetrazol-5- yl)biphenyl-4-yl]cyclo- propanecarboxamide, as a mixture of trans enantiomers | ¹H NMR (500 MHz, DMSO-d6) δ [ppm] 1.38 (ddd, 1H), 1.51 (ddd, 1H), 2.08 (s, 3H), 2.39-2.46 (m, 2H), 3.76 (s, 3H), 6.77-6.80 (m, 1H), 6.81-6.84 (m, 1H), 6.94 (d, 1H), 7.20-7.25 (m, 2H), 7.33-7.39 (m, 2H), 7.43 (d, 1H), 8.29-8.33 (m, 1H), 10.33 (s, 1H). LCMS (method 2): Rt = 1.29; MS (ESIpos) m/z = 478 (M + H)⁺. |
| 58 | 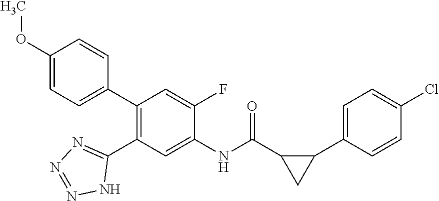 [SMILES: COc1ccc(-c2cc(F)c(NC(=O)C3CC3c3ccc(Cl)cc3)cc2-c2nnn[nH]2)cc1] | 2-(4-Chlorophenyl)-N-[5- fluoro-4′-methoxy-2-(1H- tetrazol-5-yl)biphenyl-4- yl]cyclopropanecarbox- amide, as a mixture of trans enantiomers | ¹H NMR (400 MHz, DMSO-d6) δ [ppm] 1.36-1.40 (m, 1H), 1.49-1.53 (m, 1H), 1.39-1.44 (m, 2H), 3.74 (s, 3H), 6.83 (d, 2H), 7.02 (d, 2H), 7.24 (d, 2H), 7.33-7.38 (m, 1H), 7.37 (d, 2H), 8.20 (d, 1H), 10.24 (s, 1H). LCMS (method 2): Rt = 1.23; MS (ESIpos) m/z = 464 (M + H)⁺. |
| 59 | 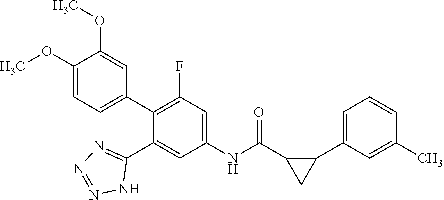 | N-[2-Fluoro-3′,4′- dimethoxy-6-(1H-tetrazol- 5-yl)biphenyl-4-yl]-2-(3- methylphenyl)cyclopro- panecarboxamide, as a mixture of trans enantiomers | ¹H NMR (500 MHz, DMSO-d6) δ [ppm] 10.72 (s, 1H), 7.82 (dd, J = 12.0, 2.0 Hz, 1H), 7.69-7.64 (m, 1H), 7.24-7.14 (m, 1H), 7.07-6.95 (m, 3H), 6.88 (d, J = 8.9 Hz, 1H), 6.65-6.55 (m, 2H), 3.74 (s, 3H), 3.59 (s, 3H), 2.41-2.34 (m, 1H), 2.28 (s, 3H), 2.11-2.03 (m, 1H), 1.56-1.48 (m, 1H), 1.46-1.37 (m, 1H). LCMS (Analytical Method D): Rt = 4.23 mins; MS (ESIpos) m/z = |
| | | | 474.05 (M + H)⁺. |
| 60 | 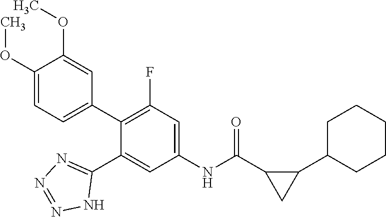 [SMILES: COc1ccc(-c2c(F)cc(NC(=O)C3CC3C3CCCCC3)cc2-c2nnn[nH]2)cc1OC] | 2-Cyclohexyl-N-[2- fluoro-3′,4′-dimethoxy- 6-(1H-tetrazol-5- yl)biphenyl-4-yl]cyclopro- panecarboxamide, as a mixture of trans enantiomers | ¹H NMR (500 MHz, DMSO-d6) δ [ppm] 10.59 (s, 1H), 7.81 (dd, J= 12.1, 2.1 Hz, 1H), 7.66 (s, 1H), 6.88 (d, J = 8.9 Hz, 1H), 6.60 (d, J = 3.9 Hz, 2H), 3.74 (s, 3H), 3.59 (s, 3H), 1.80-1.65 (m, 4H), 1.63- 1.56 (m, 2H), 1.28-0.92 (m, 7H), 0.83-0.72 (m, 2H). LCMS (Analytical Method D): Rt = 4.43 mins; MS (ESIpos) m/z = 466.15 (M + H)⁺. |
| 61 | 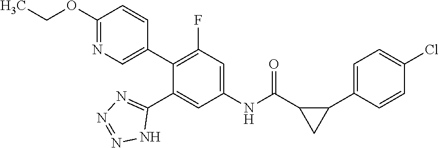 [SMILES: CCOc1ccc(-c2c(F)cc(NC(=O)C3CC3c3ccc(Cl)cc3)cc2-c2nnn[nH]2)cn1] | 2-(4-chlorophenyl)-N-[4- (6-ethoxypyridin-3-yl)-3- fluoro-5-(1H-tetrazol-5- yl)phenyl]cyclopropane- carboxamide, as a mixture of trans enantiomers | ¹H NMR (500 MHz, DMSO-d6) δ [ppm] 10.85 (s, 1H), 7.88 (d, J = 2.2 Hz, 1H), 7.86 (dd, J = 11.9, 1.9 Hz, 1H), 7.79 (s, 1H), 7.41 (dd, J = 8.5, 2.2 Hz, 1H), 7.36 (d, J = 8.5 Hz, 2H), 7.25 (d, J = 8.5 Hz, 2H), 6.76 (d, J = 8.5 Hz, 1H), 4.29 (q, J = 7.0 Hz, 2H), 2.47-2.42 (m, 1H), 2.15- 2.07 (m, 1H), 1.59-1.51 (m, 1H), |
| | | | 1.48-1.40 (m, 1H), 1.31 (t, J = 7.0 |
| | | | Hz, 3H). |
| | | | LCMS (Analytical Method F): Rt = |
| | | | 3.76 mins; MS (ESIPos): m/z = |
| | | | 479.1 (M + H)⁺. |
| 62 | 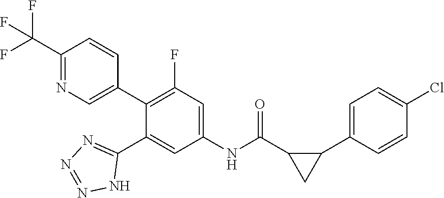 [SMILES: O=C(Nc1cc(F)c(-c2ccc(C(F)(F)F)nc2)c(-c2nnn[nH]2)c1)C1CC1c1ccc(Cl)cc1] | 2-(4-chlorophenyl)-N-{3- fluoro-5-(1H-tetrazol-5- yl)-4-[6- (trifluoromethyl)pyridin- 3-yl]phenyl}cyclopro- panecarboxamide, as a mixture of trans enantiomers | ¹H NMR (500 MHz, DMSO-d6) δ [ppm] 10.90 (s, 1H), 8.57 (s, 1H), 8.04-7.80 (m, 4H), 7.37 (d, J = 8.5 Hz, 2H), 7.26 (d, J = 8.5 Hz, 2H), 2.48-2.42 (m, 1H), 2.16-2.07 (m, 1H), 1.61-1.52 (m, 1H), 1.50-1.42 (m, 1H). LCMS (Analytical Method F): Rt = 3.85 mins; MS (ESIPos) m/z = 503.1 (M + H)⁺. |
| 63 | 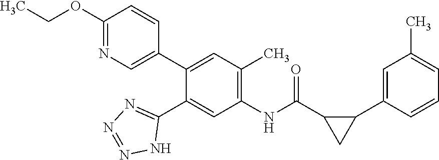 [SMILES: CCOc1ccc(-c2cc(C)c(NC(=O)C3CC3c3cccc(C)c3)cc2-c2nnn[nH]2)cn1] | N-[4-(6-Ethoxypyridin- 3-yl)-2-methyl-5-(1H- tetrazol-5-yl)phenyl]-2- (3-methylphenyl)cyclo- propanecarboxamide, as a mixture of trans enantiomers | ¹H NMR (500 MHz, DMSO-d6) δ [ppm] 1.31 (t, J = 7.0 Hz, 3H), 1.34-1.43 (m, 1H), 1.48 (dt, J = 9.2, 4.1 Hz, 1H), 2.25-2.41 (m, 8H), 4.29 (q, J = 7.0 Hz, 2H), 6.71 (d, J = 8.5 Hz, 1H), 6.96-7.04 (m, 3H), 7.19 (t, J = 7.6 Hz, 1H), 7.33 (dd, J = 8.5, 2.4 Hz, 1H), 7.42 (s, 1H), 7.92 (d, J = 2.5 Hz, 1H), 7.97 (s, 1H), 9.76 (s, 1H). |
| | | | LCMS (Analytical Method F): Rt = |
| | | | 3.57 mins; MS (ESIPos) m/z = |
| | | | 455.2 (M + H)⁺. |
| 64 | 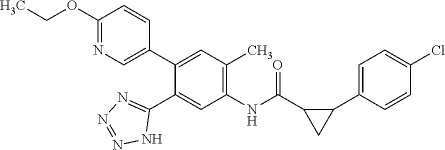 [SMILES: CCOc1ccc(-c2cc(C)c(NC(=O)C3CC3c3ccc(Cl)cc3)cc2-c2nnn[nH]2)cn1] | 2-(4-Chlorophenyl)-N-[4- (6-ethoxypyridin-3-yl)-2- methyl-5-(1H-tetrazol-5- yl)phenyl]cyclopropane- carboxamide, as a mixture of trans enantiomers | ¹H NMR (500 MHz, DMSO-d6) δ [ppm] 1.31 (t, J = 7.0 Hz, 3H), 1.35-1.43 (m, 1H), 1.46-1.54 (m, 1H), 2.28-2.34 (m, 1H), 2.35 (s, 3H), 2.39-2.45 (m, 1H), 4.29 (q, J = 7.0 Hz, 2H), 6.71 (d, J = 8.5 Hz, 1H), 7.24 (d, J = 8.4 Hz, 2H), 7.31- 7.39 (m, 3H), 7.41 (s, 1H), 7.92 (d, J = 2.3 Hz, 1H), 7.95 (s, 1H), 9.76 |
| | | | (s, 1H). |
| | | | LCMS (Analytical Method D): Rt = |
| | | | 4.48 mins; MS (ESIPos) m/z = |
| | | | 475.05 (M + H)⁺. |
| 65 | 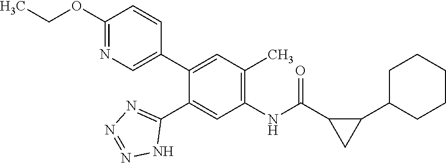 [SMILES: CCOc1ccc(-c2cc(C)c(NC(=O)C3CC3C3CCCCC3)cc2-c2nnn[nH]2)cn1] | 2-Cyclohexyl-N-[4-(6- ethoxypyridin-3-yl)-2- methyl-5-(1H-tetrazol-5- yl)phenyl]cyclopropane- carboxamide, as a mixture of trans enantiomers | ¹H NMR (500 MHz, DMSO-d6) δ [ppm] 0.67-0.83 (m, 2H), 0.90-1.01 (m, 1H), 1.01-1.25 (m, 6H), 1.31 (t, J = 7.0 Hz, 3H), 1.53-1.88 (m, 6H), 2.36 (d, J = 4.1 Hz, 3H), 4.29 (q, J = 7.0 Hz, 2H), 6.71 (d, J = 8.5 Hz, 1H), 7.30-7.36 (m, 1H), 7.41 (s, 1H), 7.81-8.02 (m, 2H), 9.61 (s, 1H). LCMS (Analytical Method F): Rt = |
| | | | 3.84 mins; MS (ESIPos): m/z = |
| | | | 447.2 (M + H)⁺. |
| 66 | 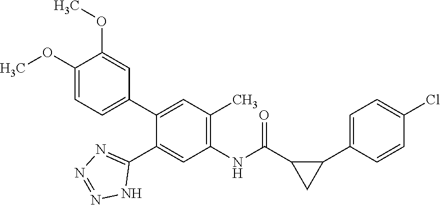 [SMILES: COc1ccc(-c2cc(C)c(NC(=O)C3CC3c3ccc(Cl)cc3)cc2-c2nnn[nH]2)cc1OC] | 2-(4-Chlorophenyl)-N- [3′,4′-dimethoxy-5- methyl-2-(1H-tetrazol-5- yl)biphenyl-4-yl]cyclopro- panecarboxamide, as a mixture of trans enantiomers | ¹H NMR (500 MHz, DMSO-d6) δ [ppm] 1.34-1.42 (m, 1H), 1.47-1.53 (m, 1H), 2.26-2.33 (m, 1H), 2.36 (s, 3H), 2.38-2.45 (m, 1H), 3.59 (s, 3H), 3.73 (s, 3H), 6.55-6.63 (m, 2H), 6.87 (d, J = 8.1 Hz, 1H), 7.24 (d, J = 8.4 Hz, 2H), 7.36 (d, J = 8.3 Hz, 2H), 7.43 (s, 1H), 7.84 (s, 1H), 9.74 (s, 1H). LCMS (Analytical Method F): Rt = 3.46 mins; MS (ESIPos) m/z = 490.1 (M + H)⁺. |
| 67 | 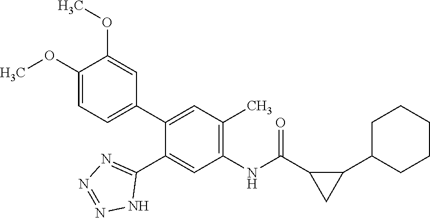 [SMILES: COc1ccc(-c2cc(C)c(NC(=O)C3CC3C3CCCCC3)cc2-c2nnn[nH]2)cc1OC] | 2-Cyclohexyl-N-[3′,4′ dimethoxy-5-methyl-2- (1H-tetrazol-5- yl)biphenyl-4-yl]cyclopro- panecarboxamide, as a mixture of trans enantiomers | ¹H NMR (500 MHz, DMSO-d6) δ [ppm] 0.68-0.84 (m, 2H), 0.93-0.98 (m, 1H), 1.01-1.30 (m, 6H), 1.56- 1.85 (m, 6H), 2.35 (s, 3H), 3.59 (s, 3H), 3.73 (s, 3H), 6.44-6.71 (m, 2H), 6.87 (d, J = 8.0 Hz, 1H), 7.42 (s, 1H), 7.77 (s, 1H), 9.58 (s, 1H). LCMS (Analytical Method F): Rt = 3.66 mins; MS (ESIPos) m/z = 462.2 (M + H)⁺. |
| 68 | 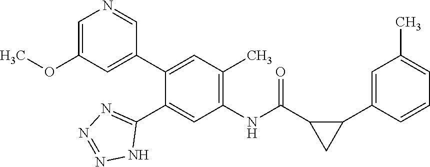 [SMILES: COc1cncc(-c2cc(C)c(NC(=O)C3CC3c3cccc(C)c3)cc2-c2nnn[nH]2)c1] | N-[4-(5-Methoxypyridin- 3-yl)-2-methyl-5-(1H- tetrazol-5-yl)phenyl]-2- (3-methylphenyl)cyclo- propanecarboxamide, as a mixture of trans enantiomers | ¹H NMR (500 MHz, DMSO-d6) δ [ppm] 9.78 (s, 1H), 8.21 (d, J = 2.6 Hz, 1H), 8.01 (s, 1H), 7.84 (s, 1H), 7.50 (s, 1H), 7.19 (t, J = 7.4 Hz, 1H), 7.10 (s, 1H), 7.03-6.97 (m, 3H), 3.75 (s, 3H), 2.37 (s, 3H), 2.36-2.31 (m, 2H), 2.29 (s, 3H), 1.48 (dt, J = 9.2, 4.6 Hz, 1H), 1.40- 1.34 (m, 1H). LCMS (Analytical Method D): Rt = |
| | | | 3.82 min; MS (ESIpos) m/z = |
| | | | 441.1 (M + H)⁺. |
| 69 | 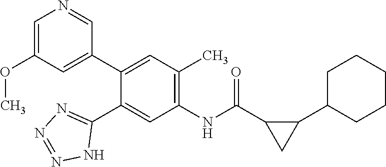 [SMILES: COc1cncc(-c2cc(C)c(NC(=O)C3CC3C3CCCCC3)cc2-c2nnn[nH]2)c1] | 2-Cyclohexyl-N-[4-(5- methoxypyridin-3-yl)-2- methyl-5-(1H-tetrazol-5- yl)phenyl]cyclopropane- carboxamide, as a mixture of trans enantiomers | ¹H NMR (500 MHz, DMSO-d6) δ [ppm] 9.65 (s, 1H), 8.21 (d, J = 2.4 Hz, 1H), 7.94 (s, 1H), 7.84 (s, 1H), 7.49 (s, 1H), 7.12-7.07 (m, 1H), 3.76 (s, 3H), 2.36 (s, 3H), 1.88-1.76 (m, 2H), 1.76-1.65 (m, 3H), 1.64- 1.56 (m, 1H), 1.25-1.03 (m, 6H), 0.97 (dt, J = 8.4, 4.0 Hz, 1H), 0.83- 0.69 (m, 2H). LCMS (Analytical Method F): Rt = |
| | | | 3.07 min; MS (ESIpos) m/z = |
| | | | 433.2 (M + H)⁺. |
| 70 | 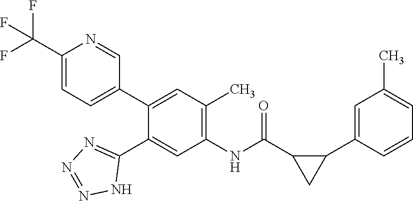 [SMILES: Cc1cccc(C2CC2C(=O)Nc2cc(-c3nnn[nH]3)c(-c3ccc(C(F)(F)F)nc3)cc2C)c1] | 2-(3-Methylphenyl)-N-{2- methyl-5-(1H-tetrazol-5- yl)-4-[6- (trifluoromethyl)pyridin- 3-yl]phenyl}cyclopro- panecarboxamide, as a mixture of trans enantiomers | ¹H NMR (250 MHz, DMSO-d6) δ [ppm] 9.81 (s, 1H), 8.55 (d, J = 1.9 Hz, 1H), 8.14 (s, 1H), 7.93-7.75 (m, 2H), 7.53 (s, 1H), 7.26-7.12 (m, 1H), 7.07-6.95 (m, 3H), 2.38 (s, 3H), 2.37-2.31 (m, 2H), 2.29 (s, 3H), 1.56-1.44 (m, 1H), 1.44-1.33 (m, 1H). LCMS (Analytical Method D): Rt = 4.46 min, MS (ESIpos) m/z = 479.05 (M + H)⁺. |
| 71 | 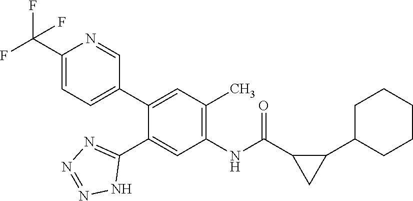 [SMILES: Cc1cc(-c2ccc(C(F)(F)F)nc2)c(-c2nnn[nH]2)cc1NC(=O)C1CC1C1CCCCC1] | 2-Cyclohexyl-N-{2- methyl-5-(1H-tetrazol-5- yl)-4-[6- (trifluoromethyl)pyridin- 3-yl]phenyl}cyclopro- panecarboxamide, as a mixture of trans enantiomers | ¹H NMR (500 MHz, DMSO-d6) δ [ppm] 9.71 (s, 1H), 8.55 (s, 1H), 8.07 (s, 1H), 7.86 (d, J = 8.1 Hz, 1H), 7.79 (d, J = 8.0 Hz, 1H), 7.52 (s, 1H), 2.38 (s, 3H), 1.88-1.76 (m, 2H), 1.75-1.64 (m, 3H), 1.62 (s, 1H), 1.29-1.02 (m, 6H), 1.02-0.92 (m, 1H), 0.86-0.69 (m, 2H). LCMS (Analytical Method D): Rt = 4.65 min, MS (ESIpos) m/z = 471.2 (M + H)⁺. |
| 72 | 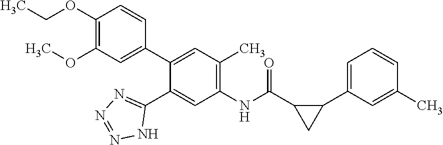 [SMILES: CCOc1ccc(-c2cc(C)c(NC(=O)C3CC3c3cccc(C)c3)cc2-c2nnn[nH]2)cc1OC] | N-[4′-Ethoxy-3′-methoxy- 5-methyl-2-(1H-tetrazol- 5-yl)biphenyl-4-yl]-2-(3- methylphenyl)cyclopro- panecarboxamide, as a mixture of trans enantiomers | ¹H NMR (500 MHz, DMSO-d6) δ [ppm] 1.25-1.39 (m, 4H), 1.41-1.51 (m, 1H), 2.23-2.40 (m, 8H), 3.59 (s, 3H), 3.98 (q, J = 6.9 Hz, 2H), 6.58 (d, J = 8.8 Hz, 2H), 6.86 (d, J = 8.1 Hz, 1H), 7.00 (t, J = 10.7 Hz, 3H), 7.18 (t, J = 7.4 Hz, 1H), 7.43 (s, 1H), 7.85 (s, 1H), 9.71 (s, 1H). |
| | | | LCMS (Analytical Method F): Rt = |
| | | | 3.60 mins; MS (ESlpos) mlz = 484 |
| | | | (M + H)⁺. |
| 73 | 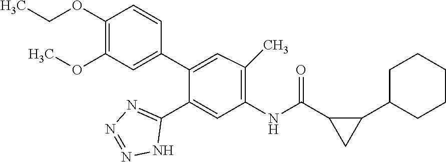 [SMILES: CCOc1ccc(-c2cc(C)c(NC(=O)C3CC3C3CCCCC3)cc2-c2nnn[nH]2)cc1OC] | 2-Cyclohexyl-N-[4′- ethoxy-3′-methoxy-5- methyl-2-(1H-tetrazol-5- yl)biphenyl-4-yl]cyclopro- panecarboxamide, as a mixture of trans enantiomers | ¹H NMR (500 MHz, DMSO-d6) δ [ppm] 0.65-0.85 (m, 2H), 0.89-1.24 (m, 7H), 1.31 (t, J = 7.0 Hz, 3H), 1.51-1.87 (m, 6H), 2.35 (s, 3H), 3.59 (s, 3H), 3.98 (q, J = 7.0 Hz, 2H), 6.54-6.63 (m, 2H), 6.86 (d, J = 8.1 Hz, 1H), 7.43 (s, 1H), 7.77 (s, 1H), 9.59 (s, 1H). LCMS (Analytical Method F): Rt = 3.84 mins; MS (ESIpos) m/z = 476 |
| | | | (M + H)⁺. |
| 74 | 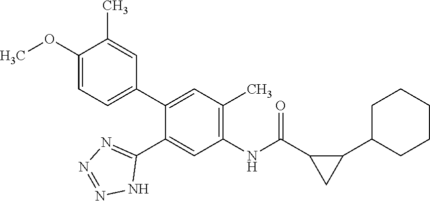 [SMILES: COc1ccc(-c2cc(C)c(NC(=O)C3CC3C3CCCCC3)cc2-c2nnn[nH]2)cc1C] | 2-Cyclohexyl-N-[4′- methoxy-3′,5-dimethyl-2- (1H-tetrazol-5- yl)biphenyl-4-yl]cyclopro- panecarboxamide, as a mixture of trans enantiomers | ¹H NMR (500 MHz, DMSO-d6) δ [ppm] 9.57 (s, 1H), 7.75 (s, 1H), 7.35 (s, 1H), 6.92 (d, J = 1.9 Hz, 1H), 6.81 (d, J = 8.5 Hz, 1H), 6.75 (dd, J = 8.4, 2.1 Hz, 1H), 3.75 (s, 3H), 2.33 (s, 3H), 2.08 (s, 3H), 1.85-1.53 (m, 6H), 1.25-1.01 (m, 6H), 0.99-0.90 (m, 1H), 0.81-0.66 (m, 2H). LCMS (Analytical Method D): Rt = 4.78 min; MS (ESIpos) m/z = 446.2 (M + H)⁺. |
| 75 | 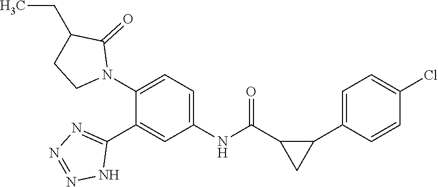 [SMILES: CCC1CCN(c2ccc(NC(=O)C3CC3c3ccc(Cl)cc3)cc2-c2nnn[nH]2)C1=O] | 2-(4-Chlorophenyl)-N-{4- [(3R)-3-ethyl-2- oxopyrrolidin-1-yl]-3- (1H-tetrazol-5- yl)phenyl}cyclopropane- carboxamide, as a mixture of stereoisomers | ¹H NMR (500 MHz, DMSO-d6) δ [ppm] 10.59 (s, 1H), 8.08 (s, 1H), 7.76 (dd, J = 8.7, 2.4 Hz, 1H), 7.43 (d, J = 8.7 Hz, 1H), 7.38-7.33 (m, 2H), 7.26-7.21 (m, 2H), 3.76 (q, J = 8.2 Hz, 1H), 3.70-3.61 (m, 1H), 2.45-2.39 (m, 1H), 2.25 (h, J = 8.6 Hz, 2H), 2.14-2.06 (m, 1H), 1.81 (tt, J = 12.7, 5.6 Hz, 1H), 1.52 (dt, J = 9.2, 4.7 Hz, 2H), 1.40 (ddd, J = 8.2, 6.3, 4.3 Hz, 1H), 1.32-1.18 (m, |
| | | | 1H), 0.83 (t, J = 7.4 Hz, 3H). |
| | | | LCMS (Analytical Method F): Rt = |
| | | | 3.27 mins; MS (ESIpos) m/z = |
| | | | 451.2 (M + H)⁺. |
| 76 | 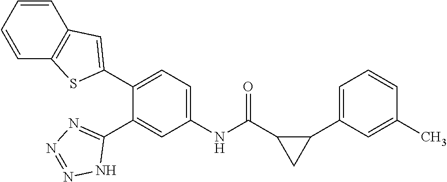 [SMILES: Cc1cccc(C2CC2C(=O)Nc2ccc(-c3cc4ccccc4s3)c(-c3nnn[nH]3)c2)c1] | N-[4-(1-Benzothiophen- 2-yl)-3-(1H-tetrazol-5- yl)phenyl]-2-(3- methylphenyl)cyclopro- panecarboxamide, as a mixture of trans enantiomers | ¹H NMR (500 MHz, DMSO-d6) δ [ppm] 10.66 (s, 1H), 7.99 (d, J = 1.9 Hz, 1H), 7.89 (d, J = 7.6 Hz, 1H), 7.86 (dd, J = 8.6, 2.2 Hz, 1H), 7.81-7.72 (m, 2H), 7.38-7.29 (m, 2H), 7.21-7.16 (m, 1H), 7.13 (s, 1H), 7.06-6.96 (m, 3H), 2.40-2.34 (m, 1H), 2.29 (s, 3H), 2.13-2.06 (m, 1H), 1.55-1.49 (m, 1H), 1.44- 1.38 (m, 1H). LCMS (Analytical Method D): Rt = |
| | | | 3.92 mins; MS (ESIpos) m/z = |
| | | | 452.1 (M + H)⁺. |
| 77 | 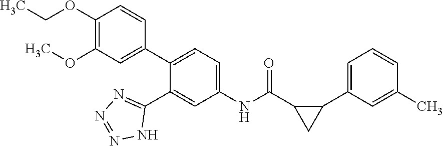 [SMILES: CCOc1ccc(-c2ccc(NC(=O)C3CC3c3cccc(C)c3)cc2-c2nnn[nH]2)cc1OC] | N-[4′-ethoxy-3′-methoxy- 2-(1H-tetrazol-5- yl)biphenyl-4-yl]-2-(3- methylphenyl)cyclopro- panecarboxamide, as a mixture of trans enantiomers | ¹H NMR (500 MHz, DMSO-d6) δ [ppm] 1.31 (t, J = 7.0 Hz, 3H), 1.34-1.44 (m, 1H), 1.46-1.56 (m, 1H), 2.04-2.14 (m, 1H), 2.29 (s, 3H), 2.32-2.41 (m, 1H), 3.58 (s, 3H), 3.98 (q, J = 7.0 Hz, 2H), 6.52- 6.63 (m, 2H), 6.85 (d, J = 8.3 Hz, 1H), 7.01 (t, J = 11.0 Hz, 3H), 7.19 (t, J = 7.5 Hz, 1H), 7.51 (d, J = 8.5 |
| | | | Hz, 1H), 7.80 (dd, J = 8.5, 2.1 Hz, |
| | | | 1H), 7.90 (s, 1H), 10.52 (s, 1H). |
| | | | LCMS (Analytical Method F): Rt = |
| | | | 3.57 mins; MS (ESIpos) m/z = 470 |
| | | | (M + H)⁺. |
| 78 | 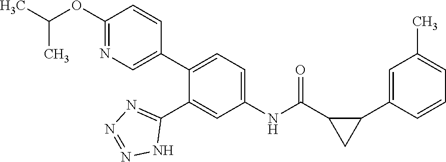 [SMILES: Cc1cccc(C2CC2C(=O)Nc2ccc(-c3ccc(OC(C)C)nc3)c(-c3nnn[nH]3)c2)c1] | N-[4-(6- isopropoxypyridin-3-yl)- 3-(1H-tetrazol-5- yl)phenyl]-2-(3- methylphenyl)cyclopro- panecarboxamide, as a mixture of trans enantiomers | ¹H NMR (500 MHz, DMSO-d6) δ [ppm] 1.28 (d, J = 6.2 Hz, 6H), 1.39 (ddd, J = 8.1, 6.4, 4.1 Hz, 1H), 1.51 (dt, J = 9.2, 4.3 Hz, 1H), 2.09 (dt, J = 9.1, 4.7 Hz, 1H), 2.28 (s, 3H), 2.36 (ddd, J = 9.4, 6.4, 4.1 Hz, 1H), 5.21 (hept, J = 6.2 Hz, 1H), 6.65 (d, J = 8.5 Hz, 1H), 7.00 (t, J = 10.9 Hz, 3H), 7.18 (t, J = 7.5 Hz, 1H), 7.28 (dd, J = 8.5, 2.1 Hz, |
| | | | 1H), 7.51 (d, J = 8.4 Hz, 1H), 7.81 |
| | | | (dd, J = 8.5, 2.2 Hz, 1H), 7.89 (d, |
| | | | J = 2.5 Hz, 1H), 8.02 (s, 1H), 10.58 |
| | | | (s, 1H). |
| | | | LCMS (Analytical Method F): Rt = |
| | | | 3.72 mins; MS (ESIPos) m/z = |
| | | | 455.1 (M + H)⁺. |
| 79 | 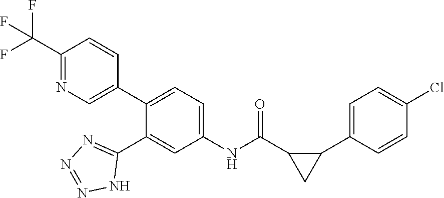 [SMILES: O=C(Nc1ccc(-c2ccc(C(F)(F)F)nc2)c(-c2nnn[nH]2)c1)C1CC1c1ccc(Cl)cc1] | 2-(4-chlorophenyl)-N-{3- (1H-tetrazol-5-yl)-4- [6-(trifluoro- methyl)pyridin-3- yl]phenyl}cyclopropane- carboxamide, as a mixture of trans enantiomers | ¹H NMR (500 MHz, DMSO-d6) δ [ppm] 10.71 (s, 1H), 8.53 (d, J = 2.0 Hz, 1H), 8.17 (s, 1H), 7.89 (dd, J = 8.5, 2.2 Hz, 1H), 7.86 (d, J = 8.0 Hz, 1H), 7.77 (dd, J = 8.1, 1.8 Hz, 1H), 7.62 (d, J = 8.5 Hz, 1H), 7.40-7.34 (m, 2H), 7.28-7.23 (m, 2H), 2.45 (m, J = 9.4, 6.3, 4.1 Hz, 1H), 2.17-2.10 (m, 1H), 1.56 (m, J = 9.3, 4.4 Hz, 1H), 1.44 (m, J = 8.2, 6.4, 4.3 Hz, 1H). LCMS (Analytical Method D): Rt = |
| | | | 4.42 mins, MS (ESIPos) m/z = |
| | | | 485.15 (M + H)⁺. |
| 80 | 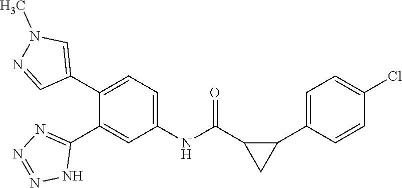 [SMILES: Cn1cc(-c2ccc(NC(=O)C3CC3c3ccc(Cl)cc3)cc2-c2nnn[nH]2)cn1] | 2-(4-chlorophenyl)-N-[4- (1-methyl-1H-pyrazol-4- yl)-3-(1H-tetrazol-5- yl)phenyl]cyclopropane- carboxamide, as a mixture of trans enantiomers | ¹H NMR (500 MHz, DMSO-d6) δ [ppm] 10.55 (s, 1H), 7.84 (s, 1H), 7.78 (s, 2H), 7.59 (d, J = 1.8 Hz, 1H), 7.35 (d, J = 8.1 Hz, 2H), 7.23 (d, J = 8.2 Hz, 2H), 5.73 (d, J = 1.9 Hz, 1H), 3.74 (s, 3H), 2.44-2.38 (m, 1H), 2.08 (dt, J = 8.6, 4.7 Hz, 1H), 1.52 (dt, J = 9.2, 4.7 Hz, 1H), 1.45-1.34 (m, 1H). LCMS (Analytical Method F): Rt = 3.15 min, m/z (ESI) = 420.1 (M + H)⁺. |
| 81 | 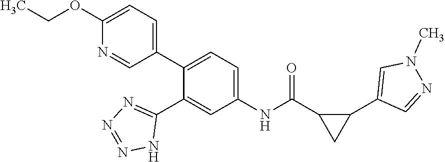 [SMILES: CCOc1ccc(-c2ccc(NC(=O)C3CC3c3cnn(C)c3)cc2-c2nnn[nH]2)cn1] | N-[4-(6-ethoxypyridin- 3-yl)-3-(1H-tetrazol-5- yl)phenyl]-2-(1-methyl- 1H-pyrazol-4-yl)cyclopro- panecarboxamide, as a mixture of trans enantiomers | (250 MHz, DMSO-d6) δ 1.11-1.26 (m, 1H), 1.31 (t, J = 7.0 Hz, 3H), 1.35-1.46 (m, 1H), 1.85-1.98 (m, 1H), 2.13-2.27 (m, 1H), 3.77 (s, 3H), 4.29 (q, J = 7.0 Hz, 2H), 6.70 (d, J = 8.0 Hz, 1H), 7.23-7.36 (m, 2H), 7.51 (d, J = 8.5 Hz, 1H), 7.57 (s, 1H), 7.82 (dd, J = 8.5, 2.3 Hz, 1H), 7.90 (d, J = 2.0 Hz, 1H), 8.02 (d, J = 2.1 Hz, 1H), 10.54 (s, 1H). |
| | | | LCMS (Analytical Method F): Rt = |
| | | | 2.47 mins, MS (ESI) m/z = 431.1 |
| | | | (M + H)⁺. |
| 82 | 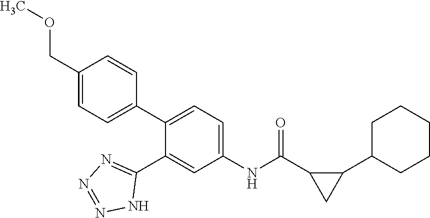 [SMILES: COCc1ccc(-c2ccc(NC(=O)C3CC3C3CCCCC3)cc2-c2nnn[nH]2)cc1] | 2-cyclohexyl-N-[4′- (methoxymethyl)-2-(1H- tetrazol-5-yl)biphenyl-4- yl]cyclopropanecarbox- amide, as a mixture of trans enantiomers | 1H NMR (500 MHz, DMSO-d6) δ 10.43 (s, 1H), 7.94 (d, J = 1.9 Hz, 1H), 7.80 (dd, J = 8.5, 2.2 Hz, 1H), 7.48 (d, J = 8.5 Hz, 1H), 7.22 (d, J = 8.1 Hz, 2H), 7.01 (d, J = 8.1 Hz, 2H), 4.38 (s, 2H), 3.28 (s, 3H), 1.83-1.55 (m, 6H), 1.24-1.02 (m, 6H), 1.01-0.95 (m, 1H), 0.80-0.72 (m, 2H). LCMS (Analytical Method D): Rt = 4.51 mins, MS (ESIpos): m/z = 432.25 (M + H)⁺. |
| 83 | 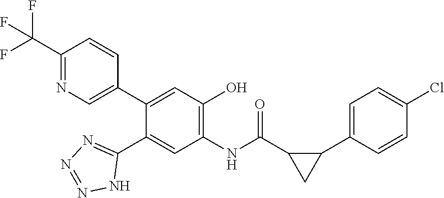 [SMILES: O=C(Nc1cc(-c2nnn[nH]2)c(-c2ccc(C(F)(F)F)nc2)cc1O)C1CC1c1ccc(Cl)cc1] | 2-(4-chlorophenyl)-N-[2- hydroxy-5-(1H-tetrazol-5- yl)-4-[6-(trifluoromethyl)- 3-pyridyl]phenyl]cyclo- propanecarboxamide, as a mixture of trans enanliomers | ¹H NMR (400 MHz, DMSO-d6) δ [ppm] 10.75 (s, 1H), 9.88 (s, 1H), 8.50 (d, 1H), 8.41 (s, 1H), 7.84 (d, 1H), 7.77 (dd, 1H), 7.37 (d, 2H), 7.22-7.28 (m, 3H), 7.00 (s, 1H), 2.58-2.62 (m, 1H), 2.40-2.45 (m, 1H), 1.49-1.54 (m, 1H), 1.33-1.37 (m, 1H) LCMS (Method 2): Rt = 1.18 mins; MS (ESIneg) m/z = 498.5 (M − H)⁺. |

40 mg of Example 48 were separated into pure enantiomers by preparative chiral SFC:

### Example 84

### N-[4-(4-tert-butyl-1H-imidazol-1-yl)-3-(1H-tetrazol-5-yl)phenyl]-2-(4-chlorophenyl)cyclopropanecarboxamide, as pure enantiomer 1

### Enantiomer 1: Rₜ=3.53 min

### Example 85

### N-[4-(4-tert-butyl-1H-imidazol-1-yl)-3-(1H-tetrazol-5-yl)phenyl]-2-(4-chlorophenyl)cyclopropanecarboxamide, as pure enantiomer 2

### Enantiomer 2: Rₜ=4.60 min

Preparative method: Instrument: Sepiatec: Prep SFC100; column: Chiralpak IE 5 μm 250×30 mm; eluent A: CO₂, Eluent B: methanol+0.4 vol-% diethylamine (99%); isocratic: 38%B; flow 100.0 mL/min; temperature: 40° C.; BPR: 150 bar; MWD @254 nm.
Analytical method: Instrument: Agilent: 1260, Aurora SFC-Modul; column: Chiralpak IE 5 μm 100×4.6 mm; eluent A: CO₂, eluent B: methanol+0.2 vol-% diethylamine (99%); isocratic: 38% B; flow 4.0 mL/min; temperature: 37.5° C.; BPR: 100 bar; MWD @254 nm.

### Biological Assays

Example compounds were tested in selected biological assays one or more times. When tested more than once, data are reported as either average values or as median values, wherein
The average value, also referred to as the arithmetic mean value, represents the sum of the values obtained divided by the number of times tested, and
The median value represents the middle number of the group of values when ranked in ascending or descending order. If the number of values in the data set is odd, the median is the middle value. If the number of values in the data set is even, the median is the arithmetic mean of the two middle values.

Example compounds were synthesised one or more times. When synthesised more than once, data from biological assays represent average values or median values calculated utilising data sets obtained from testing of one or more synthetic batch.

The potency to inhibit the Bradykinin B1 receptors was determined for the example compounds of this invention in a cell-based fluorescent calcium-mobilisation assay. The assay measures the ability of example compounds to inhibit Bradykinin B1 receptor agonist-induced increase of intracellular free Ca²⁺ in the cell line expressing B1 receptor. Specifically, calcium indicator-loaded cells are pre-incubated in the absence or presence of different concentrations of example compounds followed by the stimulation with a selective B1 receptor agonist peptide. The change of the intracellular Ca²⁺ concentration is monitored with a fluorescent plate reader FLIPR (Molecular Devices).

Calcium Flux Assays (FLIPR) with Cells Expressing Bradykinin B1 Receptor
Calcium Flux Assay (FLIPR) with Recombinant Cells for Bradykinin B1 Receptor Antagonist, Either in the Presence (hB1 IC₅₀) or Absence (hB1 free IC₅₀) of 0.1% Bovine Serum Albumin (BSA) in Assay Buffer

CHO-K1 cell line expressing human B1 receptor was purchased from Euroscreen (Gosselies, Belgium, with reference name hB1-D1). The cells were grown in Nutrient Mixture Ham's F12 (Sigma) containing 10% Foetal bovine serum (Sigma) and 400 μg/mL G418 (Sigma), 5 μg/mL puromycin (Sigma).

Notably, example compounds were tested in the FLIPR assays either in the presence (hB1 IC50) or absence (hB1 free IC50) of 0.1% BSA in assay buffer, in order to assess the potency shifts due to serum protein binding of compounds.

For the calcium flux assay, 80% confluent cells were detached from the culture vessels with Versene (Gibco), and seeded into 384-well plates (Cell binding Surface; Corning, N.Y.; #3683) at a density of 15,000 cells per well. Cells were seeded in a volume of 50 μL in medium without antibiotics and incubated overnight in a humidified atmosphere with 5% CO₂ at 37° C. The following day, the medium was replaced with 20 μL of 5 μM Fluo-4AM dye (Molecular Probes) in assay buffer (2.5 mM probenicid, 1 mg/mL pluronic acid, 135 mM NaCl, 5 mM KCl, 1.8 mM CaCl, 1 mM MgCl₂, 10 mM HEPES, 5.6 mM glucose, and 0.05% gelatine, pH 7.4), which contains or lacks 0.1% BSA for determination of compound potency units as IC₅₀ or free IC₅₀, respectively. The calcium indicator loaded cells were incubated at 37° C. for 2 hrs. Extracellular dye was then removed and each well was filled with 45 μL of assay buffer. Cell plates were kept in dark until used. Example compounds were assayed at 8 concentrations in triplicate. Serial 10-fold dilutions in 100% DMSO were made at a 100-times higher concentration than the final concentration, and then diluted 1:10 in assay buffer. 5 μL of each diluted compound was added to the well of cell plates (yielding final concentration with 1% DMSO), and incubated for 30 min at 28° C. before the addition of B1 receptor agonist on the FLIPR instrument.

Agonist plates contained the agonist Lys-(Des-Arg)-Bradykinin (Bachem, Brackley) at 3.5×EC₉₀ in assay buffer with 1% DMSO. The addition of agonist 20 μl per well to the assay plate was carried out on the FLIPR instrument while continuously monitoring Ca²⁺-dependent fluorescence at 538 nm. A peptide antagonist Lys-(Des-Arg-Leu)-Bradykinin (Bachem, Brackley) at 20 μM was used to determine the full inhibition as control.

Peak fluorescence was used to determine the response to agonist obtained at each concentration of example compound by the following equation:

% Response=100*(RFU_{(example compound)}−RFU_{(control)}/(RFU_{(DMSO)}−RFU_{(control)}

Control=full inhibition by the peptide antagonist Lys-(Des-Arg-Leu)-Bradykinin at 20 μM

The response values were plotted against the logarithm of the compound concentrations. The compounds were tested in triplicates per plate and mean values were plotted in Excel XLfit to determine IC₅₀ values, percentage of maximal inhibition and the Hill slopes.

Calcium Flux Assay (FLIPR) with Human Fibroblasts for Bradykinin B1 Receptor Antagonist (hB1 IMR-90 IC₅₀)

The Calcium flux Assay was carried out utilising IMR-90 human foetal lung fibroblasts (American Type Culture Collection, Rockville, Md.; and Coriell Institute, Camden, N.J.), which express native human B1 receptors after induction with human IL-1□.

The fibroblasts were cultured in complete growth media comprised of Dulbecco's modified Eagle's medium (DMEM; Sigma) containing 10% foetal bovine serum, 4 mM L-glutamine, and 1% nonessential amino acids. The cells were maintained in a humidified atmosphere with 5% CO₂ at 37° C. and were sub-cultured at a ratio of 1:3, every other day.

For the assay, IMR-90 fibroblasts were harvested using TrypLE Express (GIBCO/Invitrogen) and seeded into 384-well plates (Corning Cellbinding Surface, Cat. 3683) at a density of 15000 cells/well. The following day, cells were treated with 0.35 ng/mL human IL-1□ in 10% FBS/MEM for 3 h to up-regulate B1 receptors. Induced cells were loaded with fluorescent calcium indicator by incubation with 2.5 μM Fluo-4/AM (Invitrogen) at 37° C., 5% CO₂ for 2 h in the presence of 2.5 mM probenecid in 1% FBS/MEM. Extracellular dye was removed by washing with assay buffer (2.5 mM probenecid and 0.1% BSA in 20 mM HEPES/HBSS without bicarbonate or phenol red, pH 7.5). Example compounds were assayed at 8 concentrations in triplicate. After addition of example compounds to the cell plate and incubation for 30 min at 28° C., the addition of B1 agonist Lys-(Des-Arg)-Bradykinin (Bachem, Brackley) at a final concentration of EC90 was carried out on the FLIPR instrument while continuously monitoring Ca²⁺-dependent fluorescence at 538 nm. A peptide antagonist Lys-(Des-Arg-Leu)-Bradykinin (Bachem, Brackley) at 20 82 M was used to determine the full inhibition as control. IC50 values were determined by the same way described for the FLIPR assay with recombinant cells.

| | hB1 | | hB1 |
|---|---|---|---|
| Example | free IC₅₀ | hB1 | IC₅₀ IMR90 |
| Number | [nM] | IC₅₀ [nM] | [nM] |
| 1 | | 209 | |
| 2 | | 2120 | |
| 3 | | 101 | |
| 4 | 12 | 27 | |
| 5 | | 16 | |
| 6 | 6 | 20 | |
| 7 | 6 | 19 | |
| 8 | 12 | 8 | 8 |
| 9 | | 19 | |
| 10 | | 33 | |
| 11 | | 95 | |
| 12 | | 341 | |
| 13 | | 1050 | |
| 14 | | 27 | 20 |
| 15 | | 5 | 25 |
| 16 | | 15 | |
| 17 | 5 | 97 | 12 |
| 18 | 5 | 15 | 6 |
| 19 | 6 | | 10 |
| 20 | | 389 | |
| 21 | 14 | 22 | 22 |
| 22 | | 603 | |
| 23 | | 303 | |
| 24 | | 310 | |
| 25 | | 520 | |
| 26 | | 372 | |
| 27 | 13 | | |
| 28 | 509 | | |
| 29 | 526 | | |
| 30 | 514 | 3300 | |
| 31 | | 27 | |
| 32 | | 22 | |
| 33 | | 28 | |
| 34 | | 232 | |
| 35 | | 378 | |
| 36 | | 662 | |
| 37 | | 469 | |
| 38 | | 1470 | |
| 39 | | 1230 | |
| 40 | | 1030 | |
| 41 | | 33 | |
| 42 | | 31 | |
| 43 | 2210 | | |
| 44 | 26 | | |
| 45 | 463 | | |
| 46 | 1180 | | |
| 47 | 1220 | | |
| 48 | 10 | 25 | |
| 49 | | 1580 | |
| 50 | | 24 | |
| 51 | 3 | 11 | 8 |
| 52 | 41 | 37 | 78 |
| 53 | | 16 | |
| 54 | | 26 | |
| 55 | 19 | 10 | 22 |
| 56 | | 19 | |
| 57 | 24 | 124 | |
| 58 | | 134 | |
| 59 | | 83 | |
| 60 | | 177 | |
| 61 | 12 | | 44 |
| 62 | 947 | | |
| 63 | | 24 | |
| 64 | 15 | 37 | 63 |
| 65 | | 247 | |
| 66 | | 283 | |
| 67 | | 430 | |
| 68 | | 336 | |
| 69 | | 447 | |
| 70 | 157 | 86 | |
| 71 | 308 | 263 | |
| 72 | | 38 | |
| 73 | | 226 | |
| 74 | | 299 | |
| 75 | 458 | | |
| 76 | 249 | | |
| 77 | | 12 | |
| 78 | | 10 | 9 |
| 79 | 71 | 6460 | |
| 80 | 1310 | 12400 | |
| 81 | 2500 | 4070 | |
| 82 | | 2550 | |
| 83 | 188 | 484 | |
| 84 | 4 | | |
| 85 | 147 | | |

### Inhibitory Activity on Bradykinin B1 Receptor Agonist-Induced Secretion of IL-6 and IL-8 in Human IMR-90 Cells

The effect of the example compounds on cytokine secretion has been investigated in the human fetal lung fibroblast IMR-90 cell line. Here the induction of the cytokine secretion was induced by the Bradykinin B1 receptor agonists Lys-[Des-Arg9]Bradykinin and Sar-[D-Phe8]-des-Arg9-Bradykinin leading to the activation of the Bradykinin B1 receptor-driven signaling pathway.

IMR-90 cells were cultured in Eagle's Minimum Essential Medium (EMEM) containing 2 mM L-glutamine, 1 g/L glucose, 1.5 g/L NaHCO₃, 1 mM sodium pyruvate and non-essential amino acids (ATCC, 30-2003™) supplemented with 10% FBS (Biochrom, S0615) and 50 U/mL Penicillin, 50 μg/mL Streptomycin (PAA, P11-010). The assay was performed in EMEM and a cell density of 5×10⁻⁴ IMR-90 cells/96-well. The example compounds have been serial diluted in 100% DMSO and evaluated at 8 different concentrations within the range of 3 nM and 10 μM and a final DMSO concentration of 0.4%. The IMR-90 cells have been incubated with the respective concentration of the example compound for 30 min. The enhanced secretion of IL-6 and IL-8 was induced by the stimulation of these cells with 0.1 μM Lys-[Des-Arg9]Bradykinin (Tocris, 3225) and 0.1 μM Sar-[D-Phe8]-des-Arg9-Bradykinin (Tocris, 3230) for 5 hours at 37° C. and 5% CO₂. Further, cells have been treated with Lys-[Des-Arg9] Bradykinin and Sar-[D-Phe8]-des-Arg9-Bradykinin as neutral control and with 0.1% DMSO as inhibitor control. The amount of IL-6 and IL-8 in the supernatant was determined using the Human Prolnflammatory 9-Plex (MSD, K15007B) according to manufacturer's instruction. The cell viability was measured using the CellTiter-Glo Luminescent Assay (Promega, G7571) following the manufacturers protocol.

The example compounds were tested in triplicates per plate and the inhibitory activity was determined as the relation between neutral and inhibitor control in percent. IC50 values were calculated using the 4-parameter logistic model.

The compound Example 48 showed no effect on the cell viability of the stimulated IMR-90 cells. The effect on the secretion of IL-6 and IL-8 is shown in the following table:

| | IL-6 | IL-8 |
|---|---|---|
| | secretion | secretion |
| Example | IC₅₀ | IC₅₀ |
| 48 | 192 nM | 164 nM |

Rat CFA in vivo Model

Male Sprague Dawley rats are used. Mechanical hyperalgesia is induced by injecting 25 μL of Complete Freund's Adjuvant (CFA) into the plantar surface of one hind paw. Mechanical hyperalgesia is measured using the Pressure Application Measurement apparatus (Ugo Basile, Gemonio, Italy). Briefly, a linearly increasing pressure is applied to an area of ˜50 mm² of the plantar side of the hind paw until a behavioural response (paw withdrawal) is observed or until the pressure reached 1000 gf. The pressure at which the behavioural response occurred is recorded as the “Paw Withdrawal Threshold” (PWT). Both CFA-injected and contralateral PWTs are determined for each rat, in each treatment group and at each time point of the studies. Rats receive a first dose of example compound 1 hour before CFA injection and a second dose 24 hours later. Mechanical hyperalgesia testing is performed approximately 2 hours before CFA injection, then 2 and 4 hours after the second dose of example compound (i.e 26 and 28 hours after CFA treatment). Data are expressed as the mean±S.D. Area Under the Curve (AUC) of PWTs. Data are analysed by performing a one-way ANOVA followed by a Dunnett's post hoc test. For p values less than 0.05 the results are deemed to be statistically significant.

Rat Paw Oedema in vivo Model
Male Sprague Dawley rats, approximately 250 g body weight, are used. Rats are treated with the vehicle or the example compound. Rats receive an intraplantar injection of IL-1β (5 μg in 20 μL) and des-Arg⁹-bradykinin (DABK; 10 μg in 20 μL) at 20 and 40 minutes after compound treatment, respectively. Paw oedema is measured by water displacement using a plethysmometer (Ugo Basile, Gemonio, Italy). Paw oedema measurement is performed before compound administration (baseline) and subsequently at 20, 40 and 60 minutes after DABK injection. Paw oedema is calculated by subtracting the baseline value to post-DABK treatment values for each individual and at each time point. Data are analysed by calculating the area under the paw oedema time curve for each individual (0-60 minutes post-DABK). The effect of the example compounds, relative to that of the vehicle, is analysed by performing a one-way ANOVA followed by a Dunnett's post-hoc test. For p values less than 0.05 the results are deemed to be statistically significant. The mean and standard deviation are calculated for each treatment group for graphical representations.

1. A compound of general formula (I): 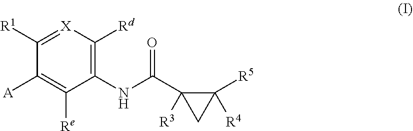
in which
A represents tetrazolyl which is attached to the rest of the molecule by the carbon atom;
R¹ represents
phenyl,
C₃-C₇-cycloalkyl,
4- to 7-membered heterocycloalkyl containing 1 or 2 heteroatoms or heteroatom-containing groups selected from NH, —NR², N, O, S, SO and SO₂,
5- to 7-membered lactam,
5- or 6-membered heteroaryl, wherein said 5-membered heteroaryl contains 1, 2 or 3 heteroatoms selected from the group consisting of S, N, NH, and O, and said 6-membered heteroaryl contains 1 or 2 N, or
bicyclic 8- to 10-membered heteroaryl containing 1, 2 or 3 heteroatoms or heteratom-containing groups selected from the group consisting of N, NH, O, S, SO, and SO₂;
wherein said R¹ is optionally substituted at one or more carbon atoms with 1 to 3 substituents R^{1a} which are the same or different wherein R^{1a} represents C₁-C₅-alkyl, C₃-C₅-cyclalkyl, —(C₁-C₃-alkyl)—(C₃-C₅-cyclalkyl), —OC₁—C₅-alkyl, —OC₃-C₅-cycloalkyl, benzyl, NHR², N(R²)₂, NH(C₃-C₅-cycloalkyl), halogen, CN, NHSO₂R², SO₂R² or 4- to 7-membered heterocycloalkyl containing 1 or 2 heteroatoms or heteroatom-containing groups selected from NH, —NR², N, O, S, SO and SO₂, and
wherein independently, if R¹ represents 4- to 7-membered heterocycloalkyl, 5- to 7-membered lactam, 5-membered heteroaryl or bicyclic 8- to 10-membered heteroaryl, each ring nitrogen atom, if present, of said R¹ is optionally substituted with a substituent R^{1b} wherein R^{1b} represents C₁-C₅-alkyl, C₃-C₅-cycloalkyl, —(C₁-C₃-alkyl)—(C₃-C₅-cycloalkyl), benzyl, SO₂R² or 4- to 7-membered heterocycloalkyl containing 1 or 2 heteroatoms or heteroatom-containing groups selected from NH, —NR², N, O, S, SO and SO₂, and
if R^{1a} represents C₁-C₅-alkyl, C₃-C₅-cycloalkyl, —(C₁-C₃-alkyl)—(C₃-C₅-cycloalkyl), —OC₁-C₅-alkyl or —OC₃—C₅-cycloalkyl and/or if R^{1b} represents C₁-C₅-alkyl, C₃-C₅-cycloalkyl or —(C₁-C₃-alkyl)—(C₃-C₅-cycloalkyl), said C₁-C₅-alkyl, C₃-C₅-cycloalkyl, —(C₁-C₃-alkyl)—(C₃-C₅-cycloalkyl), —OC₁—C₅-alkyl and —OC₃—C₅-cycloalkyl independently are optionally substituted with one or more substituents independently selected from the group consisting of OH, OR² and F, and
if R^{1a} and/or R^{1b} represent 4- to 7-membered heterocycloalkyl, each carbon atom of said 4- to 7-membered heterocycloalkyl is independently optionally substituted with one or more substituents independently selected from the group consisting of OH, OR² and F;
R² represents C₁-C₅-alkyl optionally substituted with 1 to 5 fluorine atoms;
X represents CR^{c} or N;
R^{c} represents H, halogen, CN, OH, C₁-C₅-alkyl, or —OC₁—C₅-alkyl, wherein said C₁-C₅-alkyl and —OC₁—C₅-alkyl are optionally substituted with 1 to 5 fluorine atoms;
R^{d} represents H, halogen, CN, OH, C₁-C₅-alkyl, or —OC₁—C₅-alkyl wherein said C₁-C₅-alkyl and —OC₁—C₅-alkyl are optionally substituted with 1 to 5 fluorine atoms;
R^{e} represents H, halogen, or OH;
R³ and R⁴ independently represent H or C₁-C₃-alkyl, wherein said C₁-C₃-alkyl is optionally substituted with 1 to 3 fluorine atoms;
R⁵ represents
C₁-C₅-alkyl,
(CH₂)ₚ-phenyl,
—(CH₂)ₚ-(C₅-C₇-cycloalkyl),
5- or 6-membered heteroaryl, wherein said 6-membered heteroaryl contains 1 or 2 nitrogen atoms, and wherein said 5-membered heteroaryl contains 1, 2, or 3 heteroatoms or heteroatom-containing groups, the heteroatoms being independently selected from the group consisting of S, N, NH and O, or
bicyclic 8- to 10-membered heteroaryl containing 1, 2 or 3 heteroatoms or heteroatom-containing groups selected from NH, N, O, S, SO, and SO₂,
wherein said R⁵ is optionally substituted at one or more carbon atoms with 1 to 3 substituents which are the same or different and selected from the group consisting of C₁-C₅-alkyl, —OC₁—C₅-alkyl, halogen, OH and CN, wherein said C₁-C₅-alkyl and —OC₁—C₅-alkyl independently are optionally substituted with one or more substituents independently selected from the group consisting of OH, OR² and F, and
wherein independently, if R⁵ represents a 5-membered heteroaryl or bicyclic 8- to 10-membered heteroaryl, each ring nitrogen atom, if present, of said R⁵ independently is optionally substituted with C₁-C₅-alkyl, which is optionally substituted with OH, OR² or 1 to 5 fluorine atoms; and
p is 0 or 1;
or an isomer, enantiomer, diastereomer, racemate, hydrate, solvate, or a salt thereof, or a mixture of the same.

2.-17. (canceled)
